(19) United States
(12) Patent Application Publication
(10) Pub. No.: US 2022/0211742 A1
(43) Pub. Date: Jul. 7, 2022

(54) METHODS AND COMPOSITIONS OF MiR-10 MIMICS AND TARGETS THEREOF

(71) Applicant: NEVADA RESEARCH & INNOVATION CORPORATION, Reno, NV (US)

(72) Inventor: Seungil Ro, Reno, NV (US)

(21) Appl. No.: 17/605,958

(22) PCT Filed: Apr. 24, 2020
(86) PCT No.: PCT/US20/29807

§ 371 Date: Oct. 22, 2021

Related U.S. Application Data
(60) Provisional application No. 62/837,988, filed on Apr. 24, 2019.
(60) Provisional application No. 62/964,382, filed on Jan. 22, 2020.

(51) Int. Cl. A61K 31/713; A61K 31/7105; A61P 3/10

(52) U.S. Cl. CPC A61K 31/713; A61P 3/10; A61K 31/7105

(57) ABSTRACT

The present invention relates to methods and compositions comprising a miR-10a-5p or miR-10b-5p mimic for treatment of gastrointestinal motility disorders, obesity and diabetes.

### CROSS-REFERENCE TO RELATED APPLICATIONS

The present application is entitled to priority under 35 U.S.C. § 119(e) to U.S. Provisional Patent Application No. 62/837,988, filed Apr. 24, 2019, and U.S. Provisional Patent Application No. 62/964,382, filed Jan. 22, 2020, each of which are hereby incorporated by reference in their entireties.

### STATEMENT REGARDING FEDERALLY SPONSORED RESEARCH OR DEVELOPMENT

This invention was made with government support under Grant No. DK091725 awarded by the National Institutes of Health (NIH). The government has certain rights in the invention.

### BACKGROUND OF THE INVENTION

According to data from the World Health Organization, there are over 425 million diabetic patients in the world, and that number has been rapidly increasing as the prevalence of obesity also increases. Obesity and diabetes are closely interconnected as 90% of diabetics are also obese. Type 2 diabetes (T2D), a complex and heterogeneous polygenic disease, is known as adult diabetes since it mainly occurs in adults over 40 years old. T2D accounts for up to 95% of all diagnosed cases of diabetes. Unfortunately, as of now, it has been difficult to develop a medicine that ultimately cures T2D because the cause and induction mechanisms of T2D are still elusive. Interestingly, about half of diabetic patients also have gastrointestinal (GI) complications, including gastroparesis. It is known that an abnormally high blood glucose level (hyperglycemia), a hallmark sign of diabetes, leads to gastroparesis.

MicroRNAs (miRNAs) are important regulators of molecular and cellular processes. Several studies have shown that miRNAs are involved in the regulation of various cellular processes including cell differentiation, proliferation and apoptosis.

There remains a need for methods and compositions for treating and preventing T2D, obesity, diabetic fatty liver disease, and GI complications. The current invention addresses this need.

### SUMMARY OF THE INVENTION

The present invention is based on the unexpected finding that miR-10a-5p and miR-10b-5p mimics are able to reduce or reverse various conditions and morbidities associated with diabetes mellitus including insulin resistance, obesity, fatty liver disease, cardiac function, and inflammation. The present invention is also based on the unexpected finding that treatment with miR-10a-5p and miR-10b-5p mimics are able to restore normal function in intramuscular interstitial cells of Cajal within the muscle layers of the alimentary tract and ameliorate gastrointestinal motility disorders.

Accordingly, in certain aspects, the invention provides a method of treating diabetes in a subject in need thereof, the method comprising administering to the subject an effective amount of a miR-10a-5p mimic, thereby treating the diabetic condition.

In various embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

In various embodiments, the diabetes is type 2 diabetes. In one embodiment, the miR-10a-5p mimic is mammalian.

In various embodiments, the miR-10a-5p mimic is human.

In various embodiments, the miR-10a-5p mimic is engineered.

In various embodiments, the miR-10a-5p mimic further comprises a pharmaceutically acceptable carrier or adjuvant.

In another aspect, the invention provides a method for reducing body weight in a subject, the method comprising administering an effective amount of a miR-10a-5p mimic, thereby reducing body weight in the subject.

In another aspect, the invention provides a method for lowering blood glucose in a subject, the method comprising administering an effective amount of a miR-10a-5p mimic, thereby lowering blood glucose in the subject.

In another aspect, the invention provides a method for increasing insulin sensitivity comprising administering an effective amount of a miR-10a-5p mimic to a subject in need thereof, thereby increasing insulin sensitivity in the subject.

In another aspect, the invention provides a method of treating diabetes-related fatty liver disease in a subject in need thereof comprising administering to the subject an effective amount of a miR-10a-5p mimic thereby treating the diabetes-related fatty liver disease.

In another aspect, the invention provides a method of reducing diabetes-related inflammation in a subject in need thereof comprising administering to the subject an effective amount of a miR-10a-5p mimic thereby reducing the diabetes-related inflammation.

In certain exemplary embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

In various embodiments, the miR-10a-5p mimic is mammalian.

In various embodiments, the miR-10a-5p mimic is human.

In various embodiments, the miR-10a-5p mimic is engineered.

In various embodiments, the miR-10a-5p mimic further comprises a pharmaceutically acceptable carrier or adjuvant.

In another aspect, the invention provides a method for treating gastrointestinal disease comprising administering an effective amount of a miR-10a-5p mimic to a subject in need thereof, thereby treating gastrointestinal disease in the subject.

In various embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

In various embodiments, the miR-10a-5p mimic is mammalian.

In various embodiments, the miR-10a-5p mimic is human.

In various embodiments, the gastrointestinal disease is selected from the group consisting of gastroparesis, functional gastrointestinal disorder, functional gastrointestinal motility disorder and intestinal pseudo obstruction.

In various embodiments, the functional gastrointestinal disorder is selected from the group consisting of irritable bowel syndrome, functional constipation and unspecified functional bowel disorder.

In another aspect, the invention provides a composition comprising a miR-10a-5p mimic and a pharmaceutically acceptable carrier or adjuvant.

In various embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

In various embodiments, the miR-10a-5p mimic is engineered.

In certain exemplary embodiments, the miR-10a-5p mimic comprises a nucleic acid sequence comprising SEQ ID NO: 1, 2, 3, 4, 5 or combinations thereof.

In another aspect, the invention provides a method for increasing interstitial cells of Cajal (ICC) proliferation comprising administering an effective amount of a miR-10b-5p mimic to a subject in need thereof, thereby increasing ICC proliferation in the subject.

In various embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

In various embodiments, the miR-10b-5p mimic is mammalian.

In various embodiments, the miR-10b-5p mimic is human.

In various embodiments, increasing ICC proliferation in the subject restores the function of the ICC in the subject.

In various embodiments, the ICC is located in the smooth muscle of the gastrointestinal tract of the subject.

In various embodiments, the smooth muscle is located in the stomach, small intestinal or colonic smooth muscle of the subject.

In certain exemplary embodiments, the ICC comprises ICC progenitors, ICC-MY, ICC-IM, ICC-DMP, ICC-SM or ICC-SMP.

In various embodiments, the subject is human.

In another aspect, the invention provides a method for increasing KIT expression in ICC comprising administering an effective amount of a miR-10b-5p mimic to a subject in need thereof, thereby increasing KIT expression in the subject.

In various embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

In various embodiments, the miR-10b-5p mimic is mammalian.

In various embodiments, the miR-10b-5p mimic is human.

In various embodiments, increasing KIT expression in the subject restores the function of the ICC in the subject.

In another aspect, the invention provides a method for treating gastrointestinal disease comprising administering an effective amount of a miR-10b-5p mimic to a subject in need thereof, thereby treating gastrointestinal disease in the subject.

In various embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

In various embodiments, the miR-10b-5p mimic is mammalian.

In various embodiments, the miR-10b-5p mimic is human.

In various embodiments, the gastrointestinal disease is selected from the group consisting of gastroparesis, functional gastrointestinal disorder, functional gastrointestinal motility disorder and intestinal pseudo obstruction.

In various embodiments, the functional gastrointestinal disorder is selected from the group consisting of irritable bowel syndrome, functional constipation and unspecified functional bowel disorder.

In another aspect, the invention provides a method for reducing body weight comprising administering an effective amount of a miR-10b-5p mimic to a subject in need thereof, thereby reducing body weight in the subject.

In another aspect, the invention provides a method for lowering blood glucose comprising administering an effective amount of a miR-10b-5p mimic to a subject in need thereof, thereby lowering blood glucose in the subject

In another aspect, the invention provides a method for increasing KIT⁺ pancreatic stem cell (PSC) or KIT⁺ pancreatic progenitor cell (PPC) proliferation comprising administering an effective amount of a miR-10b-5p mimic to a subject in need thereof, thereby increasing PSC or PPC proliferation in the subject.

In another aspect, the invention provides a method for increasing insulin sensitivity comprising administering an effective amount of a miR-10b-5p mimic to a subject in need thereof, thereby increasing insulin sensitivity in the subject.

In another aspect, the invention provides a method for treating diabetes comprising administering an effective amount of a miR-10b-5p mimic to a subject in need thereof, thereby treating diabetes in the subject.

In various embodiments, the diabetes is type 1 or type 2 diabetes.

In another aspect, the invention provides a method for increasing KIT expression in PSC or PPC by comprising administering an effective amount of a miR-10b-5p mimic to a subject in need thereof, thereby increasing KIT expression.

In another aspect, the invention provides a method for increasing expression of INSR, IRS2 and IRS1 in skeletal muscle cells (SkMC) comprising administering an effective amount of a miR-10b-5p mimic to a subject in need thereof, thereby increasing INSR, IRS2 and IRS1 expression.

In another aspect, the invention provides a method for reducing diabetes-related inflammation in a subject in need thereof comprising administering an effective amount of a miR-10b-5p mimic, thereby reducing the diabetes-related inflammation.

In certain exemplary embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

In various embodiments, the miR-10b-5p mimic is mammalian.

In various embodiments, the miR-10b-5p mimic is human.

In various embodiments, the miR-10b-5p mimic is engineered.

In various embodiments, the miR-10b-5p mimic further comprises a pharmaceutically acceptable carrier or adjuvant.

In another aspect, the invention provides a composition comprising a miR-10b-5p mimic and a pharmaceutically acceptable carrier or adjuvant.

In various embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

In various embodiments, the miR-10b-5p mimic is engineered.

In certain exemplary embodiments of the previous aspects or any other aspect of the present invention, the miR-10b-5p mimic comprises a nucleic acid sequence comprising SEQ ID NO: 6, 7, 8, 9, 10, 12, 13 or combinations thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1I illustrate that miR-10a-5p and miR-10b-5p are absent in jejunal and colonic KIT⁺ ICC in diabetic Kit^{copGFP/+};Lep^{ob/ob} male mice. FIG. 1A shows gross anatomical changes of Kit^{copGFP/+};Lep^{ob/ob} (diabetes) male mice at 4, 8, and 12 weeks, compared to Kit^{copGFP/+};Lep^{+/+} (wild type: WT) mice. FIG. 1B shows progressive increases of body weight in diabetic Lep mutant mice compared to WT mice. FIG. 1C shows increased blood glucose levels in diabetic mice. Diabetic mice become vastly hyperglycemic between 11-13 weeks. FIG. 1D is an automated western blot of KIT in the jejunum and colon of diabetic Lep mutant and WT mice. FIG. 1E is a Pearson correlation analysis between miRNA-seq data obtained from colonic and jejunal ICCs (CICC and JICC, respectively) isolated and pooled from diabetic Kit^{copGFP/+};Lep^{ob/ob} (n=30), non-diabetic Kit^{copGFP/+};Lep^{ob/+} (n=20) and Kit^{copGFP/+};Lep^{+/+} mice (n=20). FIGS. 1F and 1G are a heat maps of the most dynamically regulated miRNAs in diabetic and non-diabetic jejunal and colonic ICC. Both jejunal and colonic KIT⁺ ICC were isolated by FACS and a miRNA expression profile was obtained by miRNA-seq. miR-10a-5p and miR-10b-5p are highly expressed in both WT JICC and CICC, but depleted in both diabetic ICC. FIGS. 1H and 1I show expression levels of 10 prominently expressed miRNAs in FIG. 1F (boxed in black) and FIG. 1G obtained by miRNA-seq.

FIGS. 2A-2F illustrate generation of KIT⁺ (ICC specific) mir-10b knockout mice. FIG. 2A is a genomic map of tamoxifen inducible Kit^{CreERT2/+};mir-10b^{LacZ/LacZ} and Kit^{CreERT2/+};mir-10b^{-/-} mice. mir-10b^{LacZ-FLP-lox/+} mice were crossed with Kit^{CreERT2/+} mice or ROSA26^{FLP/+} mice to generate Kit^{CreERT2/+};mir-10b^{LacZ/LacZ} (resulting in mir-10b KO with tamoxifen and mir-10b WT with oil) mice and ROSA26^{FLP/+};mir-10b^{lox/lox} mice. ROSA26^{FLP/+};mir-10b^{lox/lox} mice were further crossed with Kit^{CreERT2/+} mice to generate Kit^{CreERT2/+};10b^{lox/lox} (resulting in mir-10b KO with tamoxifen and mir-10b WT with oil) mice. FIG. 2B shows gross anatomical images of male mir-10b KO (injected with tamoxifen) and mir-10b WT (injected with oil) mice. FIG. 2C shows validation of miR-10b depletion in mir-10b KO mice. Expression levels of miR-10b-5p were measured in mir-10b KO and WT jejunal and colon smooth muscle by qPCR. FIG. 2D shows Western blots of proteins regulated by, or associated with, Mir10b. KIT was decreased in jejunum and colon of mir-10b KO while KLF11 was increased in KO tissues. HOXD3 and HOXD4 had no noticeable change. GAPDH was used as a loading control. Protein ladder (L) is shown with molecular weight (kDa). FIG. 2E shows reduction of ICC (CD117⁺CD45⁻) in stomach, jejunum and colon from mir-10b KO, compared to mir-10b WT mice. FIGS. 2F-2G show cross sections, whole mount optical stacks and separated images (ICC-MY, ICC-DMP and ICC-SMP) of jejunal and/or colonic ICC (KIT⁺ ANO1⁺) in mir-10b KO mice, compared to mir-10b WT mice.

FIGS. 3A-3F illustrate that male mir-10b knockout mice develop diabetes. FIG. 3A shows body weight of mir-10b KO male mice progressively increases after 16 weeks compared to control mir-10b WT male mice. FIG. 3B shows fasting blood glucose levels of mir-10b KO male mice also progressively increase and the mice become hyperglycemic after 24 weeks. FIG. 3C shows intraperitoneal glucose tolerance test (GTT) in mir-10b KO and WT mice. Mir-10b KO mice gradually show less clearance of glucose as they age from 1 month to 7 months old. FIG. 3D shows a GTT plot of area under the curve (AUC). There is a significant increase of glucose intolerance in the 6 month and 7 month old mir-10b KO mice. FIG. 3E shows intraperitoneal insulin tolerance test (ITT) in mir-10b KO and WT mice show mir-10b KO mice progressively becoming more insensitive to insulin as mice age from 1 month to 7 months old. FIG. 3F shows an ITT plot of AUC. There is a significant increase of insulin resistance beginning in 4 month old mir-10b KO mice, compared to mir-10b WT mice that becomes exaggerated over time.

FIGS. 4A-4F illustrate that male mir-10b knockout mice display prolonged gastrointestinal (GI) transit. FIG. 4A shows total GI transit time measured using the Evans Blue method in mir-10b KO and WT mice from 1 month to 7 months of age. FIG. 4B shows gastric emptying images of 7 month old mir-10b KO and WT mice using GastroSense IVIS in vivo imaging system. FIG. 4C shows quantification of percentage of stomach emptying at 30 min. FIG. 4D shows colonic transit time measured using the bead expulsion method. FIGS. 4E-4F show an average of fecal pellet frequency and output within 24 hours.

FIGS. 5A-5G illustrate pathway analysis and target validation of miR-10b-5p in diabetes. FIG. 5A shows pathway analysis of miR-10b-5p and its target genes associated with diabetes through Ingenuity Pathway Analysis (IPA). FIG. 5B shows Western blot analyses for screening of synthetic miR-10b-5p mimics (10b duplex mimic 1-3), single stranded miR-10b-3p (10b 3p), single stranded miR-10b-5p (10b 5p), annealed miR-10b-3p and miR-10b-5p (10b 3p+5p), and mouse precursor miR-10b (mPre-10b), which may regulate KLF11 and KIT in NIH3T3 and HEK293 cells. A non-targeting RNA (scramble) and non-transfection control (NTC) were used as negative controls. Protein ladder (L) is shown with molecular weight (kDa). FIG. 5C shows validation of miR-10b-5p target proteins in NIH3T3, HEK293 cells. FIG. 5D shows NIT-1 (mβC), and Panc 10.05 cells (hβC) transfected with 10b mimic 1, miR-10b-5p antisense (10b inhibitor), two siRNAs targeting mouse Klf11 (siKlf11-1 and siKlf11-2) and human KLF11 (siKLF11-1 and siKLF11-2), scramble, and NTC. FIG. 5E shows a diagram of luciferase reporter plasmids with the miR-10b-5p target site (10b mimic binding site) of human and mouse KLF11 (hKLF11 10b TS and mKlf11 10b TS) and a mutant (mKlf11 10b TSM). FIG. 5F shows the targeting effect of 10b mimic and inhibitor in stable HEK293 cells transfected with the luciferase reporter plasmids for 6-48 hours post transfection. FIG. 5G shows target validation of KLF11 with 10b mimic and inhibitors in the four cell lines transfected with luciferase reporter plasmids and a dose dependent targeting effect of 10b mimic and inhibitor in each cell line after 12 hours.

FIGS. 6A-6J illustrate the rescue of diabetic condition and gastroparesis in male mir-10b knockout mice by miR-10b mimic injection. FIG. 6A shows body weight comparison in male diabetic mir-10b knockout (KO) mice injected with miR-10b-5p mimic (263 ng/g of body weight), mir-10b KO and WT mice given no injection. FIG. 6B shows restoration of normal blood glucose levels in mir-10b KO male mice immediately after injection with the miR-10b-5p mimic. The mice that did not receive the miR-10b mimic injection (WT and ‘No injection’ mice) did not show a decrease in glucose levels. Normal and stable glucose levels were maintained for 4 weeks post-injection. FIG. 6C shows GTT showing dramatically improved glucose tolerance in miR-10b-5p mimic treated mice. FIG. 6D shows ITT showing significantly improved insulin sensitivity in miR-10b-5p mimic treated mice. FIG. 6E shows gradual restoration of total GI transit time in miR-10b-5p mimic treated mice. FIG. 6F shows increased average fecal output in miR-10b-5p mimic treated mice. FIG. 6G shows a gastric emptying test in mir-10b KO mice injected with miR-10b-5p mimic, mir-10b KO mice and WT mice given no injection using GastroSense IVIS in vivo imaging system. FIG. 6H shows ICC in jejunum and colon are restored in mir-10 KO mice after miR-10b-5p mimic injection. FIG. 6I shows expression of KLF11 and KIT is reversed in pancreas and colon in mir-10 KO mice after miR-10b-5p mimic injection. FIG. 6J shows expression of miR-10b-5p increased in blood, pancreas, jejunum, and colon after miR-10b-5p mimic injection.

FIGS. 7A-7G illustrate that the miR-10b-5p mimic rescues the diabetic condition and slows GI transit times in high fat and high sucrose diet (HFHSD) fed mice. FIG. 7A shows the body weight comparison in HFHSD induced diabetic and normal diet (ND) male mice injected twice with miR-10b-5p mimic [Thermo Fisher Scientific, Assay ID MC11108 (Catalog #4464070)] (500 ng/g of body weight), miRNA mimic negative control, and no injection up to 15 weeks. FIG. 7B shows the restoration of normal blood glucose levels in HFHSD induced diabetic male mice immediately after injection with the miR-10b-5p mimic, but not in either the negative control or the no injection groups. Normal fasting glucose levels were maintained for 5 weeks after the 1^{st} injection and for an additional 10 weeks after the 2^{nd} injection which was similar to the normal diet male mice without treatment. In addition, normal diet fed mice injected with the miR-10b-5p mimic showed normal glucose levels, which were similar to the negative control and none injected mice. FIG. 7C highlights the results of a GTT that shows significantly improved glucose tolerance in miR-10b-5p mimic treated HFHSD mice at 2 and 4 weeks after first (1-2W and 1-4W) and second injection (2-2W and 2-4W). FIG. 7D illustrates the results of an ITT showing significantly improved insulin sensitivity in miR-10b-5p mimic treated HFHSD mice. FIG. 7E shows improvement of total GI transit time in miR-10b-5p mimic treated HFHSD diet mice at 2 and 4 weeks after the first and second injection. FIGS. 7F-7G show fecal pellet output was restored to a normal state in miR-10b-5p mimic treated HFHSD fed for 5 weeks post injection after the first and second injection.

FIGS. 8A-8G illustrate the restoration of miR-10b, its regulated proteins, ICC, and β cells in diabetic mice treated with miR-10b-5p mimic. FIG. 8A is a graph showing the amount of miR-10b-5p in the blood of normal diet (ND) healthy mice, HFHSD diabetic mice, and miR-10b-5p mimic HFHSD mice at 1-4 weeks post-injection. FIG. 8B is a graph showing the changes of insulin levels after 6 hours of fasting and after glucose injection in the three mouse groups above. FIG. 8C is a graph showing the hemoglobin A1C levels. FIG. 8D shows changes of expression of KLF11 protein in HFHSD diabetic mice and ND healthy mice treated with 10b mimic, negative control RNA, or no injection at 2 weeks. FIGS. 8E-8F shows comparison of expression levels of KLF11 and KIT in the blood, pancreas, stomach, colon, and skeletal muscle of ND, HFHSD, and miR-10b-5p mimic injected HFHSD mice at 1 and 3 weeks. FIG. 8G illustrates the images of cross sections and a whole mount showing restoration of ICC (KIT+) in stomach, jejunum, colon, and pancreas of the 3 groups at 3 week.

FIGS. 9A-9G illustrate the lack of adverse effects from miR-10b-5p mimic injection in healthy C57 mice. FIG. 9A shows a body weight comparison in healthy C57 male mice injected twice with a series of concentrations of miR-10b-5p mimic (263 ng/g, 132 ng/g, 66 ng/g, 33 ng/g of body weight) and observed for 10 weeks. FIG. 9B shows a blood glucose level comparison. FIG. 9C shows GTT showing dose-dependent improvement in glucose tolerance in the mice injected with miR-10b-5p mimic at 2 and 4 weeks after 1^{st} (1-2 weeks and 1-4 weeks) and 2^{nd} injection (2-2 Weeks and 2-4 Weeks). FIG. 9D shows ITT showing dose-dependent improvement in insulin sensitivity. FIGS. 9E-9F show fecal pellet frequency and output. FIG. 9G shows total GI transit time.

FIGS. 10A-10I illustrate that the conditional removal of KIT+ cells in male Kit^{Cre-ERT2/+};ROSA26^{DTA/+} mice leads to the diabetic condition and slow GI transit. Kit-tdTom (Kit^{Cre-ERT2/+};ROSA26^{tdTom/+}) and Kit-DTA (Kit^{Cre-ERT2/+};ROSA26^{DTA/+}) mice were treated with tamoxifen at 2 months. FIG. 10A is a graph showing that body weight of Kit-DTA male mice progressively increase after 10 weeks compared to control male mice. FIG. 10B is a graph showing that blood glucose levels of Kit-DTA male mice also progressively increases and the mice become hyperglycemic after 24 months. FIG. 10C is a graph showing the results of an intraperitoneal glucose tolerance test (GTT) in Kit-DTA and WT mice. Kit-DTA male mice gradually show less clearance of glucose at 2 and 5 months of age. FIG. 10D is a graph showing an intraperitoneal insulin tolerance test (ITT) in Kit-DTA and WT mice. Kit-DTA KO mice show progressive insulin insensitivity at 2 and 5 months. FIG. 10E shows the degeneration of β cells in Kit-DTA mice compared to Kit-tdTom mice. KIT+ cell derived (tdTom⁺) cells are found among β (INS⁺) cells in the islets in Kit-tdTom, but depleted in Kit-DTA at 7 days and 5 months post-tamoxifen injection. FIG. 10F is a graph showing the expression of miR-10b-5p measured by qPCR. FIG. 10G is a graph showing the amount of insulin and C-peptide in the blood by ELISA. FIG. 10H shows the comparison of expression levels of KLF11 and KIT in the pancreas, colon, and blood of Kit-tdTom and Kit-DTA mice after 7 days and 5 months. FIG. 10I is a graph showing the total GI transit time in Kit-DTA mice after 2 and 5 months.

FIGS. 11A-11H illustrate human patients with gastroparesis and/or T2D have similar abnormal expression patterns of miR-10b-5p, KLF11 and KIT with the mouse data obtained above. FIG. 11A shows the expression of miR-10b-5p in blood samples collected from idiopathic gastroparesis (IG, n=13), and diabetic gastroparesis (DG, n=2) patients, compared to healthy controls (HC, n=15). The IG group was divided into two groups by miR-10b higher (IG-H, n=7) and lower (IG-L, n=6). **p<0.05, ***p<0.01. FIGS. 11B and 11C shows the insulin and C-peptide in the blood of the four groups mentioned above. FIG. 11D shows the hemoglobin A1C levels. FIG. 11E shows the expression of KLF11 and KIT in the blood and stomach samples. KLF11 protein levels in the blood serum and antrum were also higher in the DG group than in the HC group while KIT levels were lower in the DG group. FIG. 11F shows the fasting blood glucose levels. FIG. 11G shows a Pearson correlation analysis between miRNA-seq data obtained from the blood of HC, IG-H, IG-L, and DG (n=2 per group). FIG. 11H shows a heat map of 70 most dynamically regulated miRNAs in HC, IG-H, IG-L, and DG (n=2 per group). miR-10a-5p and miR-10b-5p levels are highest in HC, followed by IG-H, IG-L, & DG respectively.

FIG. 12 illustrates the regulation of the diabetic condition through miR-10b. MiR-10b-5p regulates key metabolic genes (LEP, ADIPOQ, GLP1R, KIT, INS, INSR, SLC2A2, SLC2A4, IRS1, and IRS2) via two metabolically related transcriptional factors, KLF7 and KLF11, which orchestrate cell proliferation, differentiation, insulin production and sensitivity in adipocytes, ICC, pancreatic progenitor cells, beta cells, and skeletal muscle cells. In normal glucose conditions, miR-10b is abundantly expressed and inhibits KLF7 and KLF11, but the miRNA is suppressed under hyperglycemic conditions, permitting overexpression of these transcription factors, leading to diabetes, GI dysmotility and obesity.

FIGS. 13A-13B illustrate sequence comparisons of miR-10a-5p and miR-10b-5p mimics and inhibitors used in this study. FIG. 13A shows the sequence and structure of the mouse miR-10a precursor encoding miR-10a-5p and miR-10a-3p, a synthetic miR-10a-5p molecule (miR-10a-5p mimic) and a synthetic miR-10a-5p antisense molecule (miR-10a-5p inhibitor). FIG. 13B shows the sequence and structure of the mouse miR-10b precursor encoding miR-10b-5p and miR-10b-3p, a synthetic miR-10b-5p molecule (miR-10b-5p mimic) and a synthetic miR-10b-5p antisense molecule (miR-10b-5p inhibitor). Note a single nucleotide base difference between the miR-10a-5p mimic (U) and miR-10b-5p mimic (A) and also between the miR-10a-5p inhibitor (A) and miR-10b-5p inhibitor (U) are shown in bold letters.

FIGS. 14A-14B illustrate that the miR-10a-5p mimic rescues the obese and diabetic phenotype in mice fed a high-fat and high-sucrose diet (HFHSD) or a normal diet (ND). (FIGS. 14A and 14B) Body weight and fasting blood glucose comparison. Male C57 mice were fed a HFHSD for the entirety of the experiment (4-43 weeks) and injected twice (1^{st} injection at 22 weeks and 2^{nd} injection at 31 weeks) with either the miR-10a-5p mimic, negative control (a scramble RNA), or given no injection at 22 weeks and were monitored thereafter over a 21-week period, post-injection (PI). n=3-6 per group. *p<0.05 and **p<0.01 (miR-10a-5p mimic versus no injection in a HFHSD).

FIGS. 15A-16B illustrate that miR-10a-5p mimic injection results in substantial weight loss (40%) in HFHSD-induced obese and diabetic mice. (FIG. 15A) Gross anatomical changes of HFHSD-induced obese and diabetic mice injected with miR-10a-5p mimic, negative control (a scramble RNA), or no injection, compared to a ND age-matched mouse (FIG. 15B) A gross anatomical change of the same HFHSD-induced obese and diabetic mouse injected with miR-10a-5p mimic at 22 weeks before injection and at 4 weeks PI.

FIGS. 16A-16B illustrate that miR-10a-5p mimic injection reduces Body Mass Index (BMI) and waist circumference in HFHSD-induced obese and diabetic mice. Changes of average BMI and waist circumference (FIGS. 16A and 16B) in HFHSD-induced obese and diabetic mice injected with miR-10a-5p mimic, negative control (a scramble RNA), or no injection, compared to healthy mice fed a ND and give no injection in FIG. 14 (measured twice at 8 weeks PI after 1^{st} injection at 23 weeks and at 1 week PI after 2^{nd} injection at 31 weeks). n=6-12 per each group. *p<0.05 and **p<0.01.

FIG. 17 demonstrates the inverse regulation of insulin by miR-10a-5p mimic and miR-10b-5p mimic. miR-10a-5p mimic injection slightly decreases and slowly restores insulin levels in HFHSD-induced diabetic mice while the miR-10b-5p mimic injection transiently increases and gradually decreases insulin levels. Changes in insulin levels after 6 h of fasting in male mice fed a HFHSD or a ND and injected with either a miR-10a-5p mimic, miR-10b-5p mimic, or no injection over 4 weeks. n=6-12 per condition for each experiment. **p<0.01 (miR-10a-5p mimic versus no injection in a HFHSD); ##p<0.01 (miR-10b-5p mimic versus no injection in a HFHSD).

FIGS. 18A-18B illustrate that miR-10a-5p mimic injection improves glucose tolerance in HFHSD-induced diabetic mice. (FIG. 18A) Intraperitoneal (IP) glucose tolerance tests (GTT) at 4 weeks PI after 1st injection. (FIG. 18B) GTT plot of the area under the curve (AUC). n=6-12 per each group. **p<0.01.

FIGS. 19A-19B illustrate that miR-10a-5p mimic injection improves insulin tolerance in HFHSD-induced diabetic mice. (FIG. 19A) IP insulin tolerance tests (ITT) at 4W PI after 1st injection. (FIG. 19B) ITT plot of the area under the curve (AUC). n=6-12 per each group. **p<0.01.

FIGS. 20A-20B illustrate that miR-10a-5p mimic injection increases (FIG. 20A) GLP-1 and (FIG. 20B) Leptin in HFHSD-induced diabetic mice. miR-10a-5p mimic injection increases and restores (FIG. 20A) GLP-1 and (FIG. 20B) Leptin in HFHSD-induced diabetic mice to the hormone levels close to healthy mice fed a ND over 4 weeks. n=4 per each group for each experiment. **p<0.01 (miR-10a-5p mimic versus no injection in a HFHSD).

FIGS. 21A-21F illustrate that miR-10a-5p mimic injection improves GI functions. (FIG. 21A) Total GI transit time, (FIG. 21B) fecal pellet output, and (FIG. 21C) colon transit time in male diabetic mice fed a HFHSD or male healthy mice fed ND and injected with either a miR-10a-5p mimic, or no injection at 4 weeks PI. (FIG. 21D-21F) Changes of total GI transit time, fecal pellet output, and colon transit time in individual mice in FIG. 21A-C. n=6-18 per each group. **p<0.01.

FIG. 22 is a series of fluorescent images demonstrating that miR-10a-5p mimic injection improves gastric emptying. Gastric emptying was measured in male diabetic mice fed a HFHSD or male healthy mice fed a ND and injected with either a miR-10a-5p mimic or no injection, oral gavaged with a fluorescent GastroSense 750 (GS) over 60 mins in IVIS Lumina III system.

FIGS. 23A-23D illustrate that both miR-10a-5p mimic and miR-10b-5p mimic protect against an obese and diabetic phenotype in mice fed a HFHSD. Male C57 mice were injected twice (1^{st} and 2^{nd} injection) with a miR-10a-5p mimic, miR-10b-5p mimic, negative control (a scramble RNA), or no injection, and then fed a HFHSD or ND. (A and B) Body weight and fasting blood glucose in mice fed a ND. (C and D) Body weight and fasting blood glucose in mice fed a HFHSD. n=3 per each group. *p<0.05 and **p<0.01 (miR-10a-5p mimic versus no injection); #p<0.05 and ##p<0.01 (miR-10b-5p mimic versus no injection).

FIGS. 24A-24D area series of graphs showing that both miR-10a-5p inhibitor and miR-10b-5p inhibitor triggers and exacerbates the obese and diabetic phenotype in mice fed a ND and HFHSD. Male C57 mice were injected twice, once at 4 weeks and then 9 weeks (1^{st} and 2^{nd} injection) with either the miR-10a-5p inhibitor, miR-10b-5p inhibitor, both miR-10a-5p inhibitor and miR-10b-5p inhibitor (miR-10a/b-5p inhibitor), negative control (a scramble RNA), or no injection, and then fed a HFHSD or a ND. (FIGS. 24A and 24B) Body weight and fasting blood glucose in mice fed a ND. (FIGS. 24C and 24D) Body weight and fasting blood glucose in mice fed a HFHSD. n=3 per group. *p<0.05 and **p<0.01 (miR-10a-5p inhibitor versus no injection), #p<0.05 and ##p<0.01, (miR-10b-5p inhibitor versus no injection), \hat{ }p<0.05 and \hat{ }\hat{ }p<0.01 (miR-10a/b-5p inhibitor versus no injection).

FIGS. 25A-25B illustrate that miR-10a-5p inhibitor increases fasting insulin levels while miR-10b-5p inhibitor decreases fasting insulin levels in male healthy mice fed a ND and male diabetic mice fed a HFHSD at 2 weeks PI after 2^{nd} injection at 9 weeks. **p<0.01.

FIG. 26 illustrates gastric emptying images in mice fed a ND at 4 weeks PI after 2^{nd} injection with either the miR-10a-5p inhibitor, miR-10b-5p inhibitor, negative control (a scramble RNA), or no injection at 10 weeks. GS denotes GastroSense 750.

FIG. 27 is a series of Western blots demonstrating that miR-10a-5p and miR-10b-5p differentially target key proteins (LGR5, REG4, PDX1, NEROG3, and PDGFRA) in regulating stem cells or progenitor cells in colon mucosa, pancreas, and white adipocytes in mice fed a ND at 4 weeks PI after 1^{st} injection. GAPDH was used as an endogenous control in this western blot.

FIGS. 28A-28C illustrate that miR-10a-5p and miR-10b-5p differentially target the same obesity and diabetes-linked proteins. Comparison of targeting effects on obesity and diabetes-linked proteins by miR-10a-5p mimic, miR-10b-5p mimic, miR-10a-5p inhibitor and miR-10b-5p inhibitor. Mouse pancreas β-cells (NIT-1 cells) or mouse adipocytes (L-M cells) were transfected with miR-10a-5p mimic, miR-10b-5p mimic, miR-10a-5p inhibitor, miR-10b-5p inhibitor, negative control (a scramble RNA), or given no transfection. (FIG. 28A) Western blots of KLF11, KIT, LEP, GLU4, ADIPOQ, IRS-1, IRS-2, and GAPDH control in NIT-1 cells. A protein marker (M) with corresponding molecular weights (kDa) is shown. (FIG. 28B) Quantification of proteins in A normalized by GAPDH. (C) Western blots of LEP, LEPR, and GAPDH control in L-M cells. n=3 per each group. *p<0.05 and **p<0.01.

FIG. 29 is a series of images and micrographs illustrating that the miR-10a-5p mimic rescues the fatty, inflammatory, and fibrosis liver phenotype in diabetic mice fed a HFHSD. Male diabetic C57 mice fed a HFHSD or male healthy mice fed a ND for 18 weeks (4-22 weeks) were injected once at 22 weeks with miR-10a-5p mimic, miR-10a-5p inhibitor, or given no injection while continuing feeding the same diet. Gross and staining images of liver in the mice are shown. Liver tissue was dissected at 2 weeks PI from HFHSD mice and/or ND mice and stained with H&E staining, Oil Red 0 Staining (lipid), Picro Sirius Red Staining (fiber), and CD64 (macrophage).

FIGS. 30A-30B illustrate that miR-10a-5p mimic rescues liver damage in diabetic mice fed a HFHSD. (FIGS. 30A and 30B) Blood test for aspartate aminotransferase (AST) and alanine aminotransferase (ALT) in male diabetic mice fed a HFHSD or male healthy mice fed a ND for 18 weeks injected once at 22 weeks with miR-10a-5p mimic or given no injection while continuing feeding the same diet. AST and ALT was measured at 2 weeks PI from HFHSD mice and/or ND mice. n=3 per group. *p<0.05 and **p<0.01.

FIGS. 31A-31B illustrate that miR-10a-5p mimic rescues high cholesterol in diabetic mice fed a HFHSD. (FIGS. 31A and 31B) Blood test for high-density lipoprotein (HDL) and low-density lipoprotein (LDL)/very low-density lipoprotein (VLDL) cholesterol in male diabetic mice fed a HFHSD or male healthy mice fed a ND for 18 weeks and injected once at 22 weeks with miR-10a-5p mimic or given no injection while continuing feeding the same diet. HDL and LDL/VLDL were measured at 2 weeks PI from HFHSD mice and/or ND mice. n=6 per group. **p<0.01.

FIGS. 32A-32B illustrate that miR-10a-5p mimic reduces inflammation in diabetic mice fed a HFHSD. (FIGS. 32A and 32B) Blood test for pro-inflammatory cytokine, IL-6 and anti-inflammatory cytokine, IL-10 in male diabetic mice fed a HFHSD or male healthy mice fed a ND for 18 weeks and injected once at 22 weeks with miR-10a-5p mimic or given no injection while continuing feeding the same diet. IL-6 and IL-10 were measured at 2 weeks PI from HFHSD mice and/or ND mice. n=6-8 per group. **p<0.01.

FIGS. 33A-33D are a series of graphs illustrating that miR-10b-5p mimic improves heart functions in HFHSD-induced diabetic mice. Cardiac functions in male diabetic mice fed a HFHSD and male healthy mice fed a ND injected with miR-10b-5p mimic, miR-10b-5p inhibitor, or given no injection were evaluated using echocardiography. (FIG. 33A) Ejection fraction (EF). (FIG. 33B) Factional shortening (FS). (FIG. 33C) Stroke volume (SV). (FIG. 33D) Heart rate (HR). Diabetic mice fed HFHSD (no injection) showed reduced EF, FS, and SV, compared to those fed a ND (no injection). miR-10b-5p mimic injection in mice fed a ND did not change EF and FS, but improved SV. miR-10b-5p inhibitor injection in mice fed a ND reduced EF and FS, and SV. miR-10b-5p mimic or inhibitor injection in mice fed a HFHSD did not significantly change EF, FS, and SV. A heart rate of 400 to 500 beats/min was maintained in all the mice during echocardiography. n=3 per group. *p<0.05 and **p<0.01.

FIGS. 34A-34B illustrate a comparison of drug effects of miR-10a-5p, miR-10b-5p, miR-103/107 inhibitor (RG-125) on body weight and blood glucose. The miR-10a-5p mimic rescues the obese and diabetic phenotype in mice fed a HFHSD. (FIGS. 34A and 34B) Body weight and fasting blood glucose comparison. Male C57 mice were fed a HFHSD or a ND for the entirety of the experiment and injected with either the miR-10a-5p mimic, miR-10b-5p mimic, miR-103/107 inhibitor, or given no injection at 22 weeks and monitored thereafter over a 9-week period, PI. AstraZeneca collaborating with Regulus Therapeutics has completed clinical trial phase 1 and 2 using miR-103/107 inhibitor (RG-125) in patients with type 2 diabetes and non-alcoholic fatty liver disease. miR-10a-5p and miR-10b-5p have greatly improved and lasting effects in lowering blood glucose and body weight than miR-103/107 inhibitor. n=3-6 per group. *p<0.05 and **p<0.01 (miR-10a-5p mimic versus miR-103/7 inhibitor), #p<0.05 and ##p<0.01, (miR-10b-5p mimic versus miR-103/7 inhibitor).

FIG. 35 illustrates a study design of drug effects of miR-10a-5p mimic, miR-10b-5p mimic, popular anti-diabetic medications, and an FDA-approved prokinetic drug on blood glucose, body weight, and GI functions in diabetic mice fed a HFHSD over 8 weeks post treatment or healthy mice fed a ND without injection as a control. miR-10a-5p (500 ng/g body weight), miR-10b-5p (500 ng/g body weight), or scramble RNA (500 ng/g body weight) were injected twice, once at 1 week and once at 2 weeks by IP injection; Metformin (250 mg/kg body weight) or Sitagliptin (DPP4 inhibitor) (10 mg/kg body weight) was provided daily per oral (PO) for 4 weeks; Liraglutide (GLP-1 receptor agonist) (0.2 mg/kg body weight) was injected twice daily by subcutaneous injection (SC) injection for 2 weeks; Insulin (0.75 U/kg body weight) was injected once daily by IP injection for 4 weeks; Prucalopride (5-HT₄ receptor agonist) (2 mg/kg body weight) was provided daily PO for 4 weeks. Drug effects are shown in FIGS. 36-38.

FIGS. 36A-36B illustrate that miR-10a-5p mimic and miR-10b-5p mimic have better and longer effects in lowering (FIG. 36A) blood glucose and (FIG. 36B) body weight than the anti-diabetic medications and prokinetic drug described in FIG. 35. n=5 per group. *p<0.05 and **p<0.01 (miR-10a-5p mimic versus Liraglutide), #p<0.05 and ##p<0.01, (Scramble versus Liraglutide), \hat{ }p<0.05 and \hat{ }\hat{ }p<0.01 (miR-10a-5p mimic versus Liraglutide).

FIG. 37 illustrates that miR-10a-5p mimic and miR-10b-5p mimic have better and longer effects in improving GI motility than anti-diabetic medications that are used to treat chronic constipation. Total GI transit time was measured in each group at 2-8 weeks post treatment. n=5 per group. *p<0.05, **p<0.01, ***p<0.001, and ****p<0.0001. Drug treatment frequency is shown in FIG. 35.

FIGS. 38A-38B illustrate that miR-10a-5p mimic and miR-10b-5p mimic have better and longer effects in improving gastric emptying than anti-diabetic medications that are used to treat chronic are used to treat chronic constipation. FIG. 38A is a series of images demonstrating delayed gastric emptying in control scramble RNA HFHSD-fed diabetic mice is improved with miR-10a-5p mimic, miR-10b-5p mimic and Prucalopride at 4 weeks, while Liraglutide does not improve delayed gastric emptying. FIG. 38B shows gastric emptying is still normal in mice injected with miR-10a-5p mimic or miR-10b-5p mimic at 8 weeks, but it is delayed again in Prucalopride treated mice. Drug treatment frequency is shown in FIG. 35.

FIGS. 39A-39H illustrate that miR-10b-5p mimic prevents diabetes and slow GI transit in HFHSD-fed mice. FIG. 39A shows gross images of mice injected with 10b mimic, 10b inhibitor, or given no injection and fed a HFHSD or a ND. FIGS. 39B and 39C show changes of body weight and fasting blood glucose in the mice for 4 weeks PI above. FIGS. 39D and 39E show GTT and ITT. FIGS. 39F-39H show levels of A1C (FIG. 9F), miR-10b-5p (FIG. 39G), and insulin (FIG. 39H) in the mice. FIG. 39I shows total GI transit time and FIG. 39J shows total fecal pellet output.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the terms “engineered,” “genetically engineered,” “recombinant,” “non-naturally occurring,” and “non-natural” are used interchangeably to refer to synthetic polynucleotides and polypeptides that have been intentionally manipulated by humans.

As used herein “endogenous” refers to any material from or produced inside an organism, cell, tissue or system.

As used herein, the term “exogenous” refers to any material introduced from or produced outside an organism, cell, tissue or system.

As used herein, the term “RNA interference” or RNAi is the process by which synthetic small interfering RNAs (siRNAs) or the expression of an RNA molecule, including micro RNAs (miRNAs), short interfering RNAs (siRNAs), or short-hairpin RNAs (shRNAs) cause sequence-specific degradation or translational suppression of complementary mRNA molecules. As such RNAi is a form of post-transcriptional gene silencing.

As used herein, the term “miRNA mimic” refer to double-stranded, synthetic versions of endogenous miRNAs which can resemble or mimic the functions of endogenous miRNA. Synthetic miRNA mimics can be modified (e.g. chemically) to have more or less activity than their endogenous equivalent (e.g. through greater resistance to degradation). In contrast, “miRNA inhibitors” or “antimiRs” refer to synthetic, single-stranded RNA molecules which are able to bind to endogenous target miRNAs and prevent them from regulating their mRNA targets.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “knockout mouse” refers to a mouse that has had an existing gene inactivated (i.e. “knocked out”). In some embodiments, the gene is inactivated by homologous recombination. In some embodiments, the gene is inactivated by replacement or disruption with an artificial nucleic acid sequence.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

An ICC-specific miR-10b knockout (KO) mouse line was generated to study the effect of mir-10b deficiency within ICC. When the miRNA gene was conditionally deleted within the ICC of healthy adult mice, the mice surprisingly developed gastroparesis and gradually became both overweight and hyperglycemic, all hallmarks of type 2 diabetes (T2D). It has been discovered herein in the new mouse model that impaired peristaltic activities in the GI tract lead to both obesity and T2D. The most common pathological GI abnormality in diabetic patients is the depletion of ICC, which are GI pacemaker cells that regulate peristaltic activities.

This new finding of gastroparesis-induced diabetes is unexpected and contrary to the current notion that the diabetic condition causes ICC degeneration and gastroparesis. Without wishing to be bound by theory, miR-10b deficiency-mediated ICC degeneration is not just a symptom, but indeed is believed to cause obesity and T2D like symptoms.

The present invention is further based the unexpected discovery that a miR-10a-5p mimic and its regulated genes, including Krüppel-like factor 11 (KLF11), leptin (LEP), adiponectin (ADIPOQ), insulin-dependent glucose transporter type 4 (GLU4/GLUT4), and tyrosine-protein kinase (KIT), can result in reducing body fat and weight, restoring glucose homeostasis and insulin sensitivity, and improve gastrointestinal (GI) functions.

Recent studies have identified that a miR-10b-5p mimic and it's targets can be used in the treatment of diabetes and gastrointestinal motility disorders. Subsequent work identified another miRNA, miR-10a-5p mimic, that has similar effects on restoring glucose homeostasis and insulin sensitivity and in improving GI functions in diabetic mice. miR-10a-5p and miR-10b-5p differ only by a single nucleotide base in the middle of the sequence. However, injection of miR-10a-5p mimic into obese and diabetic mice drastically reduces body fat and weight in addition to lowering blood glucose and improving GI functions. Moreover, while these two miRNAs both lower blood glucose, they regulate insulin levels in opposing ways. miR-10a-5p mimic decreases insulin level while miR-10b-5p mimic increases insulin production. Consistent with the miRNA mimic data, miR-10a-5p inhibitor and miR-10b-5p inhibitor have opposite and inverse effects in insulin level. These observations suggest that unlike the miR-10b-5p mimic, which lowers blood glucose by increasing insulin production, the miR-10a-5p mimic has the ability to lower blood glucose by improving insulin sensitivity without increasing insulin. Improving insulin sensitivity by miR-10a-5p mimic may be achieved by upregulating the insulin-dependent glucose transporter GLUT4. Treatment with miR-10a-5p also increases expression of leptin, a hormone produced mainly by adipose cells and regulates the energy balance by inhibiting hunger and reduces fat storage in adipocytes. Without wishing to be bound by theory, the miR-10a-5p mimic is ideal for treatment for patients with obese type 2 diabetes and GI dysmotility, and the miR-10b-5p mimic for patients with type 1 diabetes, non-obese type 2 diabetes and GI dysmotility.

### Diabetes and Related Conditions

In one aspect, the invention of the current disclosure provides methods of treating diabetes in a subject in need thereof comprising administering to the subject an effective amount of a miR-10a-5p or miR-10b-5p mimic. Diabetes or diabetes mellitus refers to a group of diseases that are broadly related to the inability to properly regulate the use of glucose or sugar as the result of defects in the production, secretion, or function of the hormone insulin. Aberrant function of insulin leads to abnormalities of carbohydrate, fat, and protein metabolism. Diabetes is divided into two general types. Type 1, or insulin-dependent diabetes mellitus results from an autoimmune reaction that results in the destruction of insulin-producing β-islet cells in the pancreas, resulting in a systemic lack of insulin. Treatment for the disease consists mainly of regularly monitoring blood glucose levels and injecting insulin several times a day. Failure to control insulin dosage can result in severe hypoglycemia and life-threatening damage to the brain and other functions.

Type 2, or non-insulin-dependent diabetes mellitus (T2DM) is a more complex disease that typically develops in adults and is associated with glucose-responsive tissues such as adipose fat tissue, muscle, and liver that become resistant to the action of insulin. In early stages of T2DM, pancreatic islet cells compensate by secreting excess insulin. Without intervention, β-islet cell dysfunction can result, leading to decompensation and chronic hyperglycemia. Additionally, T2DM may also be accompanied by peripheral insulin resistance, wherein otherwise insulin sensitive cells fail to respond normally. Whereas Type 1 diabetes is often an acute disease that presents early in life, Type 2 diabetes can develop gradually later in life as a result of many factors including genetics and lifestyle. There are several classes of medications commonly used for the treatment of T2DM: 1) insulin release agents that directly stimulate insulin secretion but are at risk of causing hypoglycemia; 2) a diet insulin releaser that enhances glucose-induced insulin secretion but must be taken before each meal; 3) biguanides including metformin that reduce production of glucose from digestion; 4) insulin sensitizers such as thiazolidinedione derivatives rosiglitazone and pioglitazone which improve peripheral response to insulin by modulating the expression of glucose metabolism genes, but has side effects such as weight gain, edema and hepatotoxicity; 5) Insulin injection, often required in late-stage T2DM.

The metabolic nature of type 2 diabetes and the abnormally high blood sugar that results from the condition often leads to the development of symptoms and disorders that affect a wide range of body tissues. Diabetes is linked to higher incidences of obesity, fatty liver disease, hyperlipidemia, fatty liver disease, and GI motility disorders including gastroparesis and constipation.

T2DM-related insulin resistance is generally associated with atherosclerosis, obesity, hyperlipidemia and essential hypertension. This group of abnormal conditions constitutes “metabolism” or insulin resistance. In addition, insulin resistance is associated with fatty liver disease, which may lead to chronic inflammation or nonalcoholic steatohepatitis, fibrosis and cirrhosis. Nonalcoholic fatty liver disease begins with the accumulation of triacylglycerol in the liver and is defined as the presence of cytoplasmic lipid droplets in more than 5% of hepatocytes or TAG levels exceeding the 95th percentile for healthy individuals. Both T2DM and fatty liver disease are associated with adverse outcomes of the other. Type 2 diabetes is a risk factor for progressive liver disease and liver-related death in patients with fatty liver disease, whereas fatty liver disease may be a marker of cardiovascular risk and mortality in individuals with Type 2 diabetes. Nonalcoholic steatohepatitis, a histological subtype of NAFLD characterized by hepatocyte injury and inflammation, is present in approximately 10% of patients with T2DM and is associated with an increased risk for the development of cirrhosis and liver-related death.

Diabetic gastroparesis, which is a common, yet serious, chronic disorder of the upper gastrointestinal tract, is defined by the presence of delayed gastric emptying in the absence of physical obstruction and is associated with symptoms such as nausea, vomiting, early satiation, bloating, and abdominal pain. Currently, the only FDA-approved drug for diabetic gastroparesis is metoclopramide, a dopamine D2 receptor antagonist and 5-HT3 receptor antagonist with weak 5-HT4 agonist activity, which is indicated for the relief of symptoms associated with acute and recurrent diabetic gastric stasis for no longer than 12 weeks of treatment. However, metoclopramide treatment is associated with significant side effects such as sudden muscle spasms and depression/mood changes.

Gene Expression Regulation by miRNA

In one aspect, the invention provides method of using miRNA mimics of miR-10a-5p and miR-10b-5p to treat diabetes and conditions related to diabetes.

miRNAs are small, non-coding RNA molecules that are typically 20-22 nucleotides in length. miRNAs act as key regulators of gene expression and function which act to modify gene expression by interacting with post-transcription RNA and modulating its stability and subsequent translation. Understanding of the biological roles of ncRNAs, including miRNAs is advancing rapidly. Numerous evolutionary studies have revealed that non-coding RNAs could be expressed in nearly 4-fold greater quantity than protein-coding RNAs.

Endogenous miRNAs are transcribed as 100-1000 nucleotide (nt) primary miRNAs (pri-RNAs) by RNA polymerase II. miRNAs may be modified by 5′ capping and 3′ poly(A) tailing. The miRNA-encoding portion of the pri-miRNA forms a hairpin, which is cleaved by the dsRNA-specific ribonuclease Drosha and its cofactor DiGeorge syndrome critical region 8 (DGCR8), to form a pre-miRNA that is about 60-70 nt long. The pre-miRNA is further processed by Dicer and the trans-activator RNA-binding protein TRBP to yield a miRNA duplex containing two mature miRNAs (5′- and 3′-strand miRNAs). Each mature miRNA is about 22-23 nt in length.

Depending on the degree of complementarity between the mature miRNA and its target, several mechanisms of mRNA silencing can occur. The leading bases from positions 2 to 7 of the mature miRNA are termed the ‘seed’ sequence and provide most of the pairing specificity with the target mRNA. In some cases, complete pairing between the seed sequence and its cognate target is sufficient to mediate cleavage and degradation of the cognate mRNA. More typically for mammalian and viral mRNA targets, however, cleavage is impaired by mismatched pairing in the seed and other regions and translational inhibition occurs through physical interference with the binding of translational machinery. Since the complementary length of seed sequence required for miRNAs to target cognate mRNAs is short, each miRNA has the possibility to target and modulate hundreds of transcripts. Furthermore, mRNA molecules can, in turn, also be acted upon by numerous distinct miRNAs. While most miRNAs decrease target protein levels by less than 2-fold, this is often sufficient to exert a significant physiological effect. Thus, the endogenous miRNA pathway represents a highly efficient system to simultaneously fine-tune the expression of numerous genes as well as modulate specific functional pathways. miRNAs are predicted to control the activity of approximately 30% of all protein-coding genes in mammals, and play important roles in normal physiological processes ranging from embryonic development to hematopoietic cell development to diseases ranging from cardiovascular disease, cancer, and immune disorders. Recent studies have demonstrated that miR-10a-5p and miR-10b-5p mimics may successfully treat diabetes and other conditions such as gastroparesis, fatty liver disease, and obesity.

miRNA Mimics

In certain embodiments of the current disclosure, the invention provides a number of miRNA mimics that target human genes that are regulated by endogenous miR-10b-5p and miR-10a-5p miRNAs. miRNA mimics is a strategy for gene silencing that utilizes non-natural double-stranded miRNA-like RNA fragments. The 5′ end of these fragments possess a partially complementary sequence to a cognate sequence in the 3′ UTR unique to the target gene. Once expressed or introduced into cells, the RNA fragment mimics the function of the endogenous miRNA and binds specifically to target gene, which results in posttranscriptional repression of the gene, typically through inhibition of transcription. Thus, miRNA mimics are able to be precisely targeted to affect specific genes. In various embodiments of the current invention, the miR-10a-5p and miR-10b-5p mimics are able to affect the expression of a number of target genes including, but not limited to KLF11, KIT, leupeptin, GLU/GLUT4, adiponectin, LGR5, REG4, PDX1, NEUROG3, PDGFA, LEP, LEPR, and IRS21 (miR-10a-5p) and KLF7, KLF11, KIT, INSR, IRS2 and IRS1 (miR-10b-5p).

miR-10a-5p Mimics

In some embodiments of the invention, the miR-10a-5p mimic mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

The sequence and/or source of some miR-10a-5p mimics and miR-10a-5p mimic inhibitors is listed below:

10a-5p mimic 1: 10a duplex RNA
Sense:
(SEQ ID NO: 1)
5′ UACCCUGUAGAUCCGAAUUUGUG 3′
Antisense:
(SEQ ID NO: 2)
3′ TTAUGGGACAUCUAGGCUUAAAC 5′
miR-10b-5p: 10a-5p single stranded RNA
(miR-10a-5p)
(SEQ ID NO: 1)
5′ UACCCUGUAGAUCCGAAUUUGUG 3′
10a Inhibitor: 10a-3p single stranded RNA
(miR-10a-5p inhibitor)
(SEQ ID NO: 5)
5′ AUGGGACAUCUAGGCUUAAACAC 3′
Human mir-10a gene (stem-loop) with mature
sequence underlined
Accession: MI0000266
ID: hsa-mir-10a
(SEQ ID NO: 4)
5′ GAUCUGUCUGUCUUCUGUAUAUACCCUGUAGAUCCGAAU
UUGUGUAAGGAAU 3′
Mature sequence mmu-miR-10a-5p (miR-10a mimic)
Accession: MIMAT0000253
ID: hsa-miR-10a
(SEQ ID NO: 1)
UACCCUGUAGAUCCGAAUUUGUG
Mouse mir-10a gene (stem-loop) with mature
sequence underlined
Accession: MI0000685
ID: mmu-mir-10a
(SEQ ID NO: 3)
5′ GACCUGUCUGUCUUCUGUAUAUACCCUGUAGAUCCGAAU
UUGUGUAAGGAAU 3′
Mature sequence mmu-miR-10a-5p (miR-10a mimic)
Accession: MIMAT0000648
ID: mmu-miR-10a
(SEQ ID NO: 1)
UACCCUGUAGAUCCGAAUUUGUG

miR-10b-5p Mimics

In some embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p.

The sequence and/or source of some miR-10b-5p mimics and miR-10b-5p mimic inhibitors is listed below:

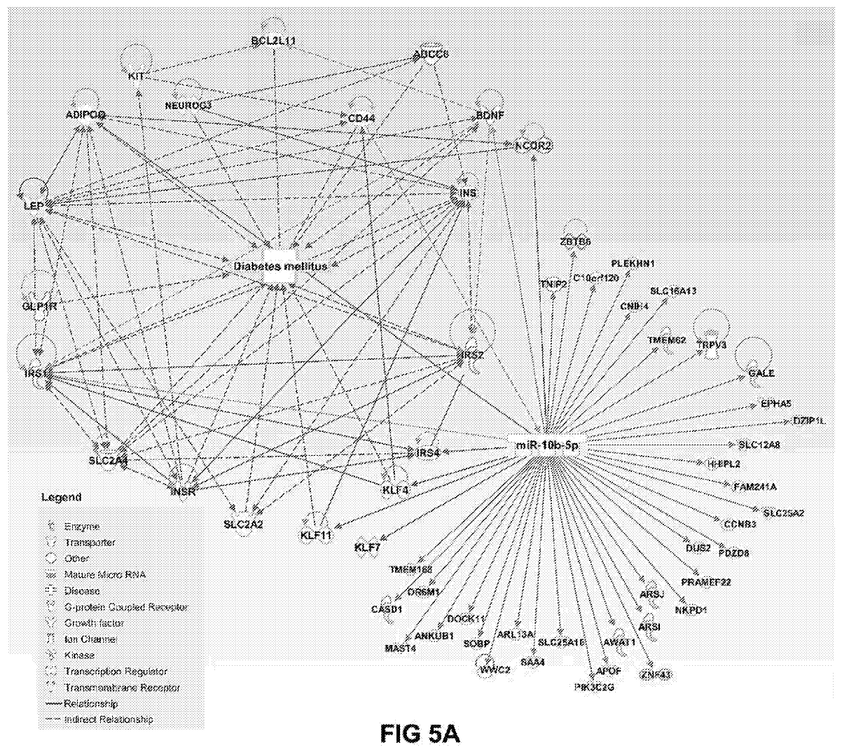

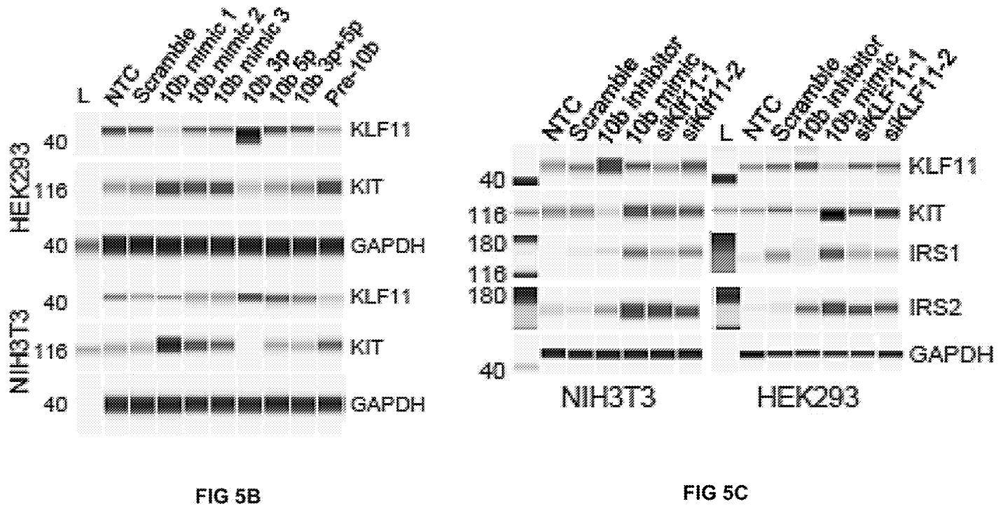

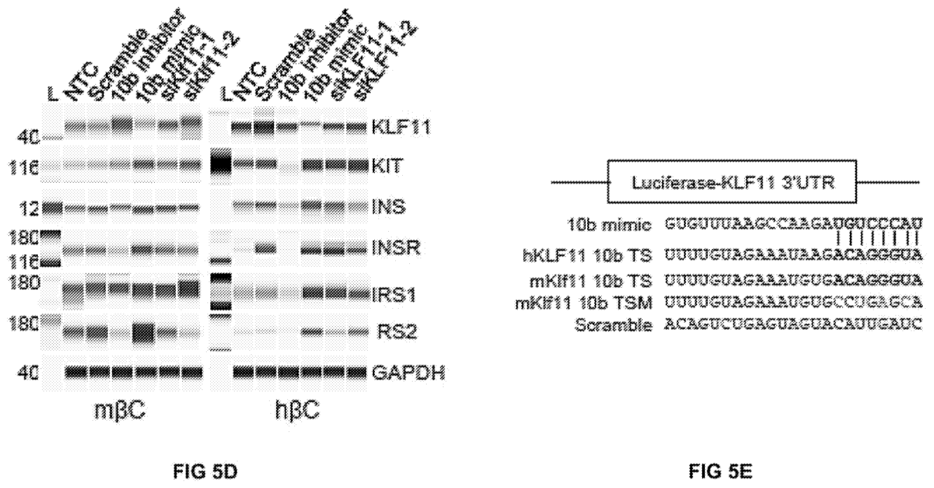

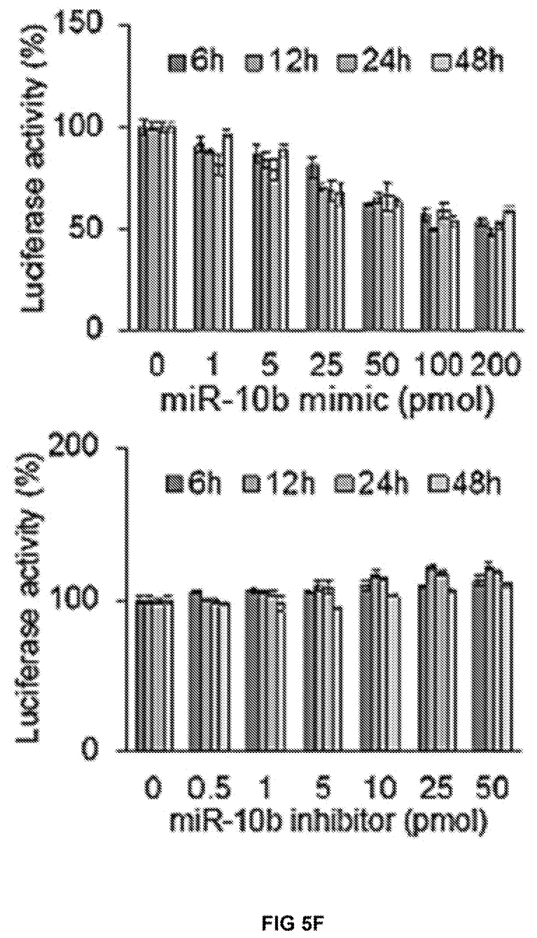

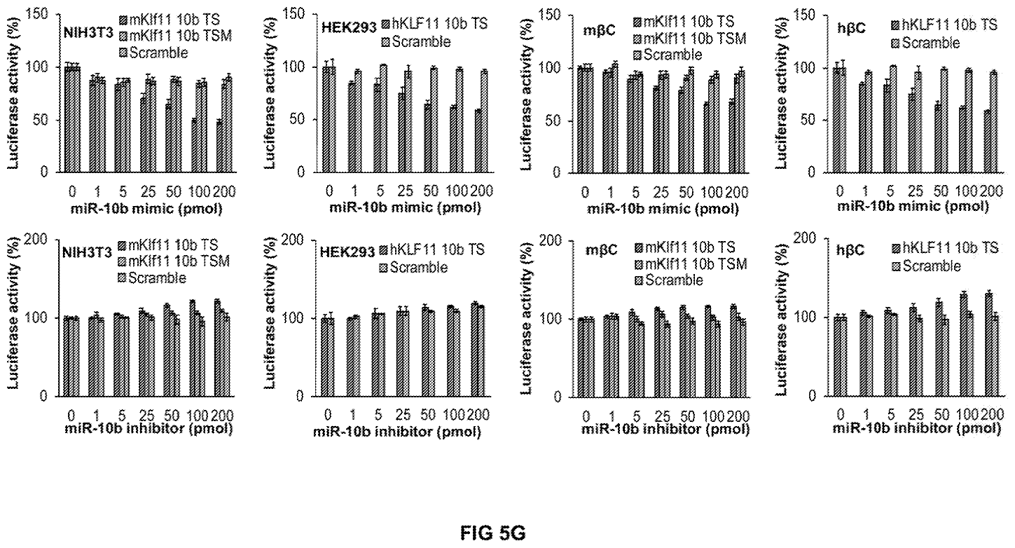

10b mimic 1 [FIGS. 5B-5C] (MC11108, Thermo
Fisher Scientific): 10b duplex RNA
Sense:
(SEQ ID NO: 6)
5′ UACCCUGUAGAACCGAAUUUGUG 3′
Antisense:
(SEQ ID NO: 7)
3′ TTAUGGGACAUCUUGGCUUAAAC 5′
10b mimic 2 [FIG. 5C] (C-310431-07-0005,
Dharmacon): 10b duplex RNA
10b mimic 3 [FIG. 5C] (219600, QIAGEN):
10b RNA duplex
miR-10b-5p (IDT): 10b-5p single stranded
RNA (miR-10b-5p)
(SEQ ID NO: 6)
5′ UACCCUGUAGAACCGAAUUUGUG 3′
Annealed miR-10b-3p and miR-10b-5p (IDT): 10b
duplex RNA with miR-10b-5p/3p
Sense:
(SEQ ID NO: 6)
5′ UACCCUGUAGAACCGAAUUUGUG 3′
Antisense:
(SEQ ID NO: 8)
3′ AUAAGGGGAUCUUAGCUUAGAC 5′
10b mimic 7 (IDT): 10b precursor RNA
(SEQ ID NO: 9)
5′ UACCCUGUAGAACCGAAUUUGUGUGGUACCCACAUAGUCACAG
AUUCGAUUCUAGGGGAAUA 3′

miRNA mimics may be commercially obtained. Human miR-10b-5p mimic (has-miR-10b-5p) may be commercially obtained from Thermo Fisher Scientific (product name mirVana® miRNA mimic; product ID MC11108).

Mature miR-10b sequence is:
(SEQ ID NO: 6)
5′-UACCCUGUAGAACCGAAUUUGUG-3′
Human mir-10b gene (stem-loop) with mature
sequence underlined
Accession Number: MI0000267
ID: hsa-mir-10b
>hsa-mir-10b MI0000267
(SEQ ID NO: 10)
CCAGAGGUUGUAACGUUGUCUAUAUAUACCCUGUAGAACCGAAUUU
GUGUGGUAUCCGUAUAGUCACAGAUUCGAUUCUAGGGGAAUAUAUG
GUCGAUGCAAAAACUUCA
Mature sequence hsa-miR-10b-5p (miR-10b mimic)
Accession: MIMAT0000254
ID: hsa-miR-10b
>hsa-miR-10b-5p MIMAT0000254
(SEQ ID NO: 6)
UACCCUGUAGAACCGAAUUUGUG
Mouse mir-10b gene (stem-loop) with mature
sequence underlined
Accession: MI0000221
ID: mmu-mir-10b
>mmu-mir-10b MI0000221
(SEQ ID NO: 11)
UAUAUACCCUGUAGAACCGAAUUUGUGUGGUACCCACAUAGUCAC
AGAUUCGAUUCUAGGGGAAUAUA
Mature sequence mmu-miR-10b-5p (miR-10b mimic)
Accession: MIMAT0000208
ID: mmu-miR-10b
>mmu-miR-10b-5p MIMAT0000208
(SEQ ID NO: 6)
UACCCUGUAGAACCGAAUUUGUG
Negative controls or inhibitors:
Negative control #1 (4464058, Thermo Fisher
Scientific): non-targeting duplex RNA
10b inhibitor (Thermo Fisher Scientific):
10b-5p inhibitor RNA (antisense of miR-10b-5p)
(SEQ ID NO: 12)
5′ CACAAAUUCGGUUCUACAGGGUA 3′
miR-10b-3p (IDT): 10b-3p single stranded RNA
(miR-10b-5p inhibitor)
(SEQ ID NO: 13)
5′ CAGAUUCGAUUCUAGGGGAAUA 3′

### Methods of Treatment

Provided is a method of treating diabetes in a subject in need thereof, the method comprising administering to the subject an effective amount of a miR-10a-5p mimic, thereby treating the diabetic condition. In some embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

In some embodiments, the diabetes is type 2 diabetes. In some embodiments, the miR-10a-5p mimic is mammalian. In some embodiments, the miR-10a-5p mimic is human. In some embodiments, the miR-10a-5p mimic is engineered. In some embodiments, the miR-10a-5p mimic further comprises a pharmaceutically acceptable carrier or adjuvant.

Also provided is a method for reducing body weight in a subject, the method comprising administering an effective amount of a miR-10a-5p mimic, thereby reducing body weight in the subject. In some embodiments, treatment with miR-10a-5p results in the upregulation of leptin, thereby decreasing hunger and reducing fat storage, thereby resulting in weight loss.

Also provided is a method for lowering blood glucose in a subject, the method comprising administering an effective amount of a miR-10a-5p mimic, thereby lowering blood glucose in the subject.

Also provided is a method for increasing insulin sensitivity comprising administering an effective amount of a miR-10a-5p mimic to a subject in need thereof, thereby increasing insulin sensitivity in the subject.

In some embodiments of any one of the previous methods, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p. In further embodiments, the miR-10a-5p mimic is mammalian. In further embodiments, the miR-10a-5p mimic is human. In further embodiments, the miR-10a-5p mimic is engineered. In further embodiments, the miR-10a-5p mimic further comprises a pharmaceutically acceptable carrier or adjuvant.

Also provided is a method for treating gastrointestinal disease comprising administering an effective amount of a miR-10a-5p mimic to a subject in need thereof, thereby treating gastrointestinal disease in the subject. In some embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p. In some embodiments, the miR-10b-5p mimic is mammalian. In some embodiments, the miR-10b-5p mimic is human.

In some embodiments, the gastrointestinal disease is selected from the group consisting of gastroparesis, functional gastrointestinal disorder, functional gastrointestinal motility disorder and intestinal pseudo obstruction. In further embodiments, the functional gastrointestinal disorder is selected from the group consisting of irritable bowel syndrome, functional constipation and unspecified functional bowel disorder.

Also provided is a method for increasing insulin sensitivity comprising administering an effective amount of a miR-10a-5p mimic to a subject in need thereof, thereby increasing insulin sensitivity in the subject. In some embodiments, administration of the miR-10a-5p mimic results in increased expression of insulin-dependent glucose transporter 4 (GLU4/GLUT4). In further embodiments, administration of the miR-10a-5p mimic results in a decrease in blood glucose levels in the subject.

In some embodiments of any one of the previous methods, the miR-10a-5p mimic targets a gene selected from the group consisting of KLF11, KIT, Leupeptin, GLU4/GLUT4, Adiponectin, and IRS2 and combinations thereof.

Also provided is a composition comprising a miR-10a-5p mimic and a pharmaceutically acceptable carrier or adjuvant. In some embodiments, the miR-10a-5p mimic and a pharmaceutically acceptable carrier or adjuvant. In some embodiments, the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p., In some embodiments, the miR-10a-5p mimic is engineered.

Provided is a method for increasing interstitial cells of Cajal (ICC) proliferation comprising administering an effective amount of a miR-10b-5p mimic to a subject in need thereof, thereby increasing ICC proliferation in the subject. In some embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p. In further embodiments, the miR-10b-5p mimic is mammalian. In yet further embodiments, the miR-10b-5p mimic is human.

In some embodiments, increasing ICC proliferation in the subject restores the function of the ICC in the subject. In further embodiments, the ICC is located in the smooth muscle of the gastrointestinal tract of the subject. In yet further embodiments, the smooth muscle is located in the stomach, small intestinal or colonic smooth muscle of the subject.

In some embodiments, the ICC comprises ICC progenitors, ICC-MY, ICC-IM, ICC-DMP, ICC-SM or ICC-SMP.

In some embodiments, the subject is human.

Provided is a method for increasing KIT expression in ICC comprising administering an effective amount of a miR-10b-5p mimic to a subject in need thereof, thereby increasing KIT expression in the subject. In some embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p. In further embodiments, the miR-10b-5p mimic is mammalian. In yet further embodiments, the miR-10b-5p mimic is human.

In some embodiments, increasing KIT expression in the subject restores the function of the ICC in the subject.

In some embodiments, administering the miR-10-b-5p mimic to the subject results in decreased expression of KLF7 and KLF11.

In some embodiments, ICC are phenotypically inactivated and become non-functional in gastrointestinal diseases.

Also provided is a method for treating gastrointestinal disease comprising administering an effective amount of a miR-10b-5p mimic to a subject in need thereof, thereby treating gastrointestinal disease in the subject. In some embodiments, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p. In further embodiments, the miR-10b-5p mimic is mammalian. In yet further embodiments, the miR-10b-5p mimic is human.

In some embodiments, the gastrointestinal disease is selected from the group consisting of gastroparesis, functional gastrointestinal disorder, functional gastrointestinal motility disorder and intestinal pseudo obstruction. In further embodiments, the functional gastrointestinal disorder is selected from the group consisting of irritable bowel syndrome, functional constipation and unspecified functional bowel disorder.

Provided is a method for reducing body weight comprising administering an effective amount of a miR-10b-5p mimic to a subject in need thereof, thereby reducing body weight in the subject.

Also provided is a method for lowering blood glucose comprising administering an effective amount of a miR-10b-5p mimic to a subject in need thereof, thereby lowering blood glucose in the subject

Also provided is a method for increasing KIT⁺ pancreatic stem cell (PSC) or KIT⁺ pancreatic progenitor cell (PPC) proliferation comprising administering an effective amount of a miR-10b-5p mimic to a subject in need thereof, thereby increasing PSC or PPC proliferation in the subject. In some embodiments, the PSC or PPC proliferation is increased in the subject as compared to a control.

Also provided is a method for increasing insulin sensitivity comprising administering an effective amount of a miR-10b-5p mimic to a subject in need thereof, thereby increasing insulin sensitivity in the subject. In some embodiments, administration of the miR-10b-5p mimic results in increased glucose intake into skeletal muscle cells (SkMC) in the subject. In further embodiments, administration of the miR-10b-5p mimic results in a decrease in blood glucose levels in the subject. In some diabetic subjects, insulin resistance may be found in the SkMC of the subject.

Also provided is a method for increasing insulin gene expression comprising administering an effective amount of a miR-10b-5p mimic to a subject in need thereof, thereby increasing insulin gene expression in the subject. In some embodiments, administration of the miR-10b-5p mimic results in restored function of beta cells, thereby increasing the amount of insulin produced from the beta cells as compared to a control. In some embodiments, administering the miR-10-b-5p mimic to the subject results in decreased expression of KLF7 and KLF11. In some embodiments, the decreased expression of KLF11 results in increased expression of NEUROG3 and INS in the beta cells.

In some untreated diabetic subjects, insulin is not produced or is only produced at low levels in the beta cells of the subject. In untreated diabetic subjects, there is a reduction of beta cells located in islet cells. Islet cells are located in the pancreas of the subject.

Also provided is a method for treating diabetes comprising administering an effective amount of a miR-10b-5p mimic to a subject in need thereof, thereby treating diabetes in the subject. In some embodiments, the diabetes is type 1 or type 2 diabetes. In some embodiments, administration of the miR-10b-5p mimic results in increased glucose intake into skeletal muscle cells (SkMC) in the subject. In further embodiments, administration of the miR-10b-5p mimic results in a decrease in blood glucose levels in the subject.

Provided is a method for increasing KIT expression in PSC or PPC by comprising administering an effective amount of a miR-10b-5p mimic to a subject in need thereof, thereby increasing KIT expression. In some embodiments, administering the miR-10-b-5p mimic to the subject results in decreased expression of KLF11.

Also provided is a method for increasing expression of INSR, IRS2 and IRS1 in skeletal muscle cells (SkMC) comprising administering an effective amount of a miR-10b-5p mimic to a subject in need thereof, thereby increasing INSR, IRS2 and IRS1 expression. In some embodiments, administering the miR-10-b-5p mimic to the subject results in decreased expression of KLF11. In some embodiments, the decreased expression of KLF11 results in increased INSR, IRS2 and IRS1 expression.

In some embodiments of any one of the previous methods, the miR-10b-5p mimic is a miR-10b-5p duplex, a chemically modified double stranded miR-10b-5p, an unmodified double stranded miR-10b-5p, a single stranded chemically modified miR-10b-5p or a single stranded unmodified miR-10b-5p. In further embodiments, the miR-10b-5p mimic is mammalian. In yet further embodiments, the miR-10b-5p mimic is human.

In some embodiments of any one of the previous methods, the miR-10b-5p mimic is engineered.

In some embodiments of any one of the previous methods, the miR-10b-5p mimic is administered by injection. In some embodiments, the miR-10b-5p mimic may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes.

In some embodiments of any one of the previous methods, the miR-10b-5p mimic further comprises a pharmaceutically acceptable carrier or adjuvant.

It is envisaged that the miR-10b-5p mimic may be administered to a subject in combination with other suitable treatments for diabetes (type 1 or type 2). In some embodiments, the other treatments are insulin, insulin sensitizers (Thiazolidinedione), metformin, glucagon like peptide-1 (GLP-1) receptor agonists (Exenatide, Albiglutide, Dulaglutide, Liraglutide, Lixisenatide), dipeptidylpeptidase-4 (DPP4) inhibitors (Sitagliptin, Vildagliptin, Alogliptin, Linagliptin), sodium-glucose transporters-2 (SGLT2) inhibitors (Dapagliflozin, Empagliflozin), and sulfonylureas (Glimepiride).

It is envisaged that the miR-10b-5p mimic may be administered to a subject in combination with other suitable treatments for a gastrointestinal disorder. In some embodiments, the other treatments are prokinetics, 5-HT4 receptor agonist (Prucalopride, tegaserod and Velusetrag), ghrelin agonist (Relamorelin), dopamine receptor antagonists and 5-HT4 agonists (metoclopramide and domperidone), motilin receptor agonists (Macrolide antibiotics: Erythromycin and azithromycin)], anti-emetic agents (Aprepitant, Promethazine, Prochlorperazine, and Ondansetron), and agents acting on secretion (Lubiprostone and Tenapanor).

In some embodiments, the miR-10b-5p mimic comprises a nucleic acid sequence comprising SEQ ID NO: 6, 7, 8, 9, 10, 12, 13 or combinations thereof.

In any one of the previous aspects or embodiments, the miR-10b-5p mimic may be chemically modified or unmodified.

### Pharmaceutical Compositions

Provided is a composition comprising a miR-10a-5p mimic or a miR-10b-5p mimic and a pharmaceutically acceptable carrier or adjuvant. In some embodiments, the miR-10b-5p or miR-10a-5p mimic is a miR-10a-5p or miR-10b-5p duplex, a chemically modified double stranded miR-10a-5p or miR-10b-5p, an unmodified double stranded miR-10a-5p or miR-10b-5p, a single stranded chemically modified miR-10a-5p or miR-10b-5p or a single stranded unmodified miR-10a-5p or miR-10b-5p. In further embodiments, the miR-10a-5p or miR-10b-5p mimic is mammalian. In yet further embodiments, the miR-10a-5p or miR-10b-5p mimic is human.

In some embodiments of any one of the previous compositions, the miR-10a-5p or miR-10b-5p mimic is engineered.

In some embodiments, the miR-10a-5p mimic comprises a nucleic acid sequence comprising SEQ ID NO: 1, 2, 4, 5 or combinations thereof.

In some embodiments, the miR-10b-5p mimic comprises a nucleic acid sequence comprising SEQ ID NO: 6, 7, 8, 9, 10, 12, 13 or combinations thereof.

In some embodiments, the miR-10a-5p or miR-10b-5p mimic may be chemically modified or unmodified.

Pharmaceutical compositions of the present invention may comprise a miR-10a-5p or miR-10b-5p mimic as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Pharmaceutical compositions of the present invention may be administered in solid or liquid form such as tablets, capsules, powders, solutions, suspensions, emulsions and the like. Pharmaceutical compositions of the present invention may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes. In some embodiments, the composition may be applied to the nose, throat or bronchial tubes, for example by inhalation.

The dosage of the above treatments to be administered to a patient will vary with the precise nature of the condition being treated and the recipient of the treatment and can be determined by physical and physiological factors such as body weight, severity of condition, previous or concurrent therapeutic interventions, and on the route of administration. The scaling of dosages for human administration can be performed according to art-accepted practices. The dose for a miR mimic, for example, will generally be in the range 1 to about 100 mg for an adult patient, usually administered monthly for a period between 1 and 12 months. The preferred monthly dose is 1 to 10 mg per month although in some instances larger doses of over 10 mg per month may be used.

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 1 microgram/kg/body weight, from 5 microgram/kg/body weight, 10 microgram/kg/body weight, 50 microgram/kg/body weight, 100 microgram/kg/body weight, 200 microgram/kg/body weight, 350 microgram/kg/body weight, 500 microgram/kg/body weight, 1 milligram/kg/body weight, 5 milligram/kg/body weight, 10 milligram/kg/body weight, 50 milligram/kg/body weight, 100 milligram/kg/body weight, 200 milligram/kg/body weight, 350 milligram/kg/body weight, or 500 milligram/kg/body weight, to 1000 mg/kg/body weight or more per administration, and any range derivable therein. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 1 micrograms compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular subject. Those of skill in the art would recognize the conditions and situations warranting modified dosing.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventor regard as his invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1—A Highly Expressed miR-10b-5p, is Absent in Jejunal and Colonic KIT⁺ ICC in Hyperglycemic Kit^{copGFP/+};Lep^{ob/ob} Male Mice

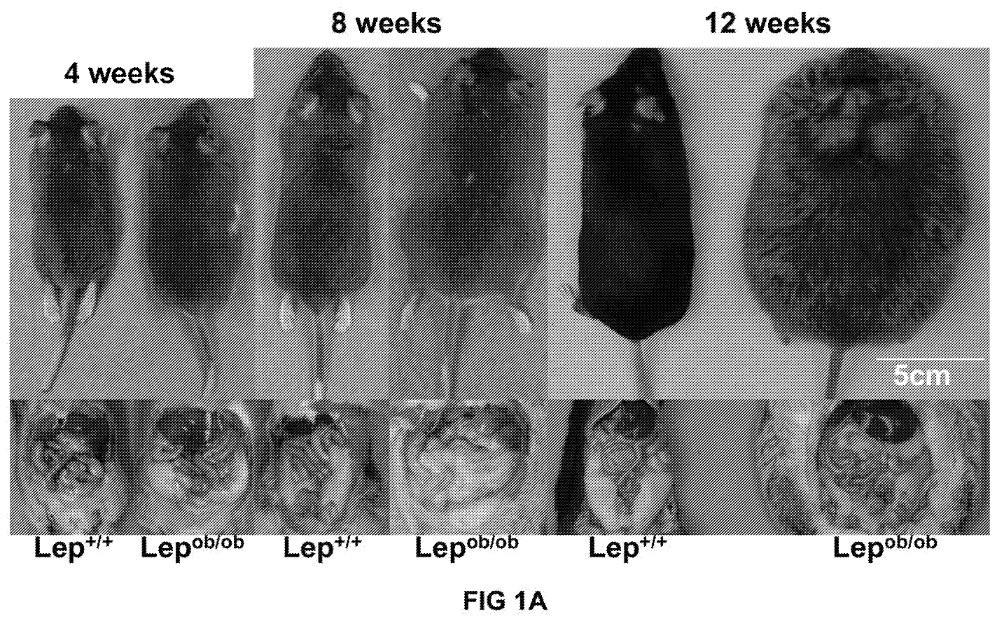

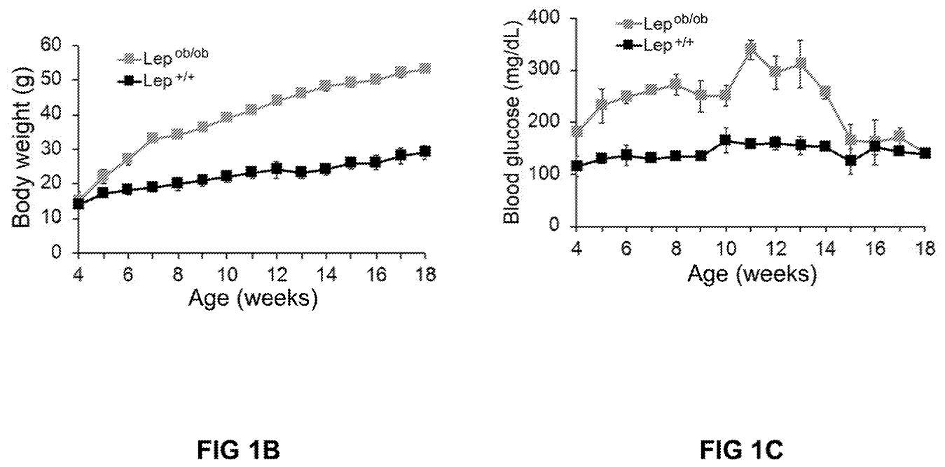

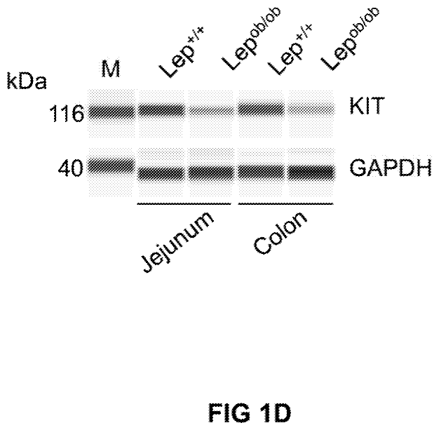

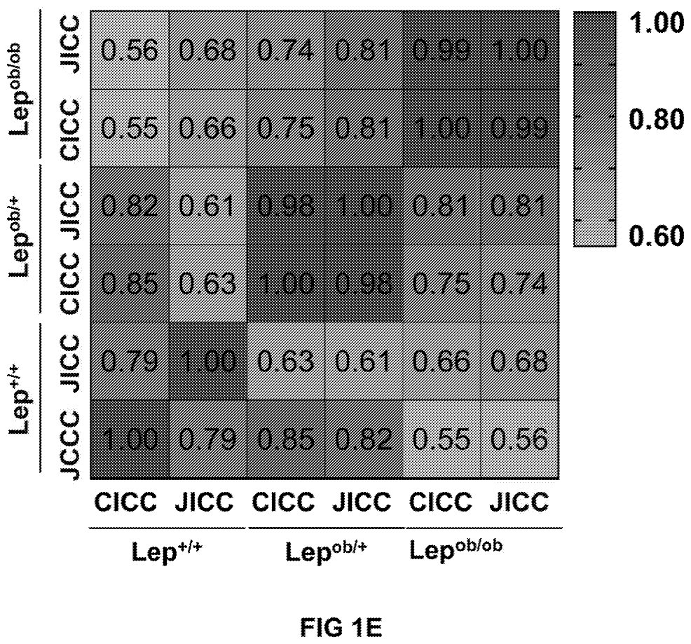

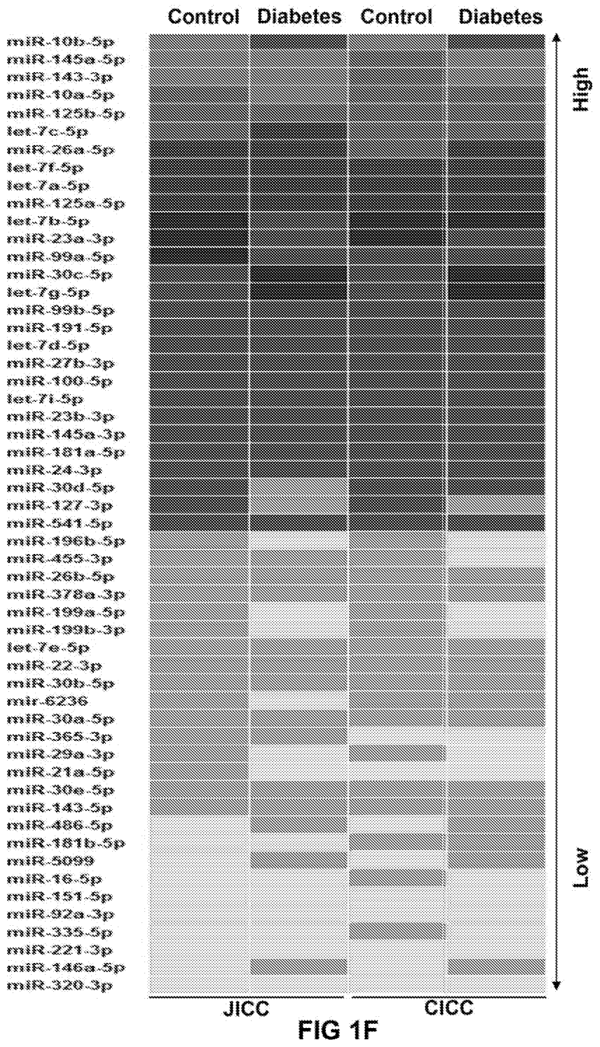

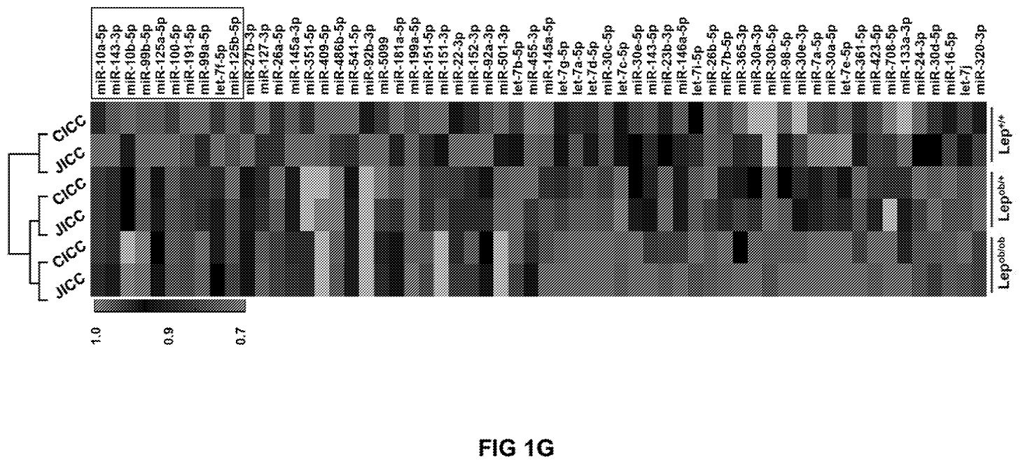

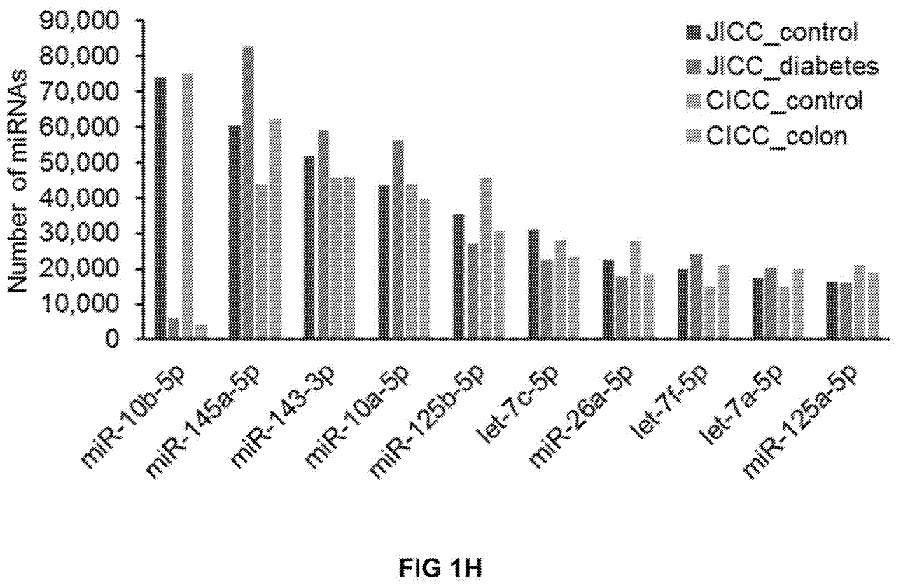

The obese female spontaneous leptin gene mutant mouse, Lep^{ob/ob} (type 2 diabetic model) (The Jackson Laboratory), was crossed with a ICC-copGFP male mouse Kit^{copGFP/+} (Ro, 2010) to generate Kit^{copGFP/+};Lep^{ob/ob} mice (Ro, 2010). The diabetic male Kit^{copGFP/+};Lep^{ob/ob} mice and healthy control male Kit^{copGFP/+};Lep^{+/+}mice at 4, 8, and 12 weeks were sacrificed and compared (FIG. 1A). The leptin mutant males contained much more body fat than the healthy controls. The leptin mutant mice gained weight faster than their wild type (WT) siblings 4 weeks after birth and their body weight continued to be twice as much as controls at 18 weeks (FIG. 1B). Fasting blood glucose levels were much higher in the mutants, compared to the healthy controls, and reached starkly hyperglycemic levels (over 250 mg/dL) after 11 weeks (FIG. 1C). However, the glucose was reduced to normal levels after 14 weeks. It has been previously reported that during the temporal hyperglycemic (diabetic) condition, the number of copGFP⁺ ICC in the small intestine and colon were reduced as they lost KIT expression (Ro, 2010). Diabetic copGFP⁺ ICC were isolated by FACS from the colon and jejunum respectively from 3 male diabetic mice at 12-13 weeks along with age and sex matched healthy non-diabetic controls. Isolated jejunal copGFP⁺ ICC (JICC) and colonic copGFP⁺ ICC (CICC) were pooled into one sample respectively which was used for small RNA isolation and subsequent miRNA sequencing. Global miRNA expression patterns within diabetic homozygous Lep mutants and non-diabetic WT Lep colonic and jejunal ICCs are quite different (Pearson correlation coefficient: 0.55 for CICC and 0.68 for JICC) while Lep heterozygous ICCs show similarity between both homozygous mutant (Pearson correlation coefficient: 0.75 for CICC and 0.81 for JICC) and WT mice (Pearson correlation coefficient: 0.63 for CICC and 0.61 for JICC) (FIG. 1E). Surprisingly, colonic and jejunal ICCs show remarkably similar miRNA expression profiles within Lep heterozygous and homozygous mutant genotypes (Pearson correlation coefficient: 0.98 and 0.99, respectively) (FIG. 1E). miR-10a-5p, miR-143-3p, and miR-10b-5p are the most highly expressed miRNAs in WT ICCs, but their expression was substantially decreased in Lep heterozygous and homozygous mutant ICCs. Among these three miRNAs, miR-10b-5p displayed a diabetes-dependent reduction as further reductions in diabetic Lep homozygous mutant ICCs were observed (FIGS. 1F and 1G). These facilitated the determination that miR-10b-5p is selectively depleted in diabetic ICCs. MiRNA sequencing and annotation showed that miR-10a-5p and miR-10b-5p was most highly expressed in healthy WT JICC and CICC, but dramatically depleted in diabetic JICC and CICC (FIGS. 1F-1G). These results are shown in FIGS. 1A-1G.

### Example 2-Generation of KIT⁺ ICC Specific Mir-10b Knockout Mice

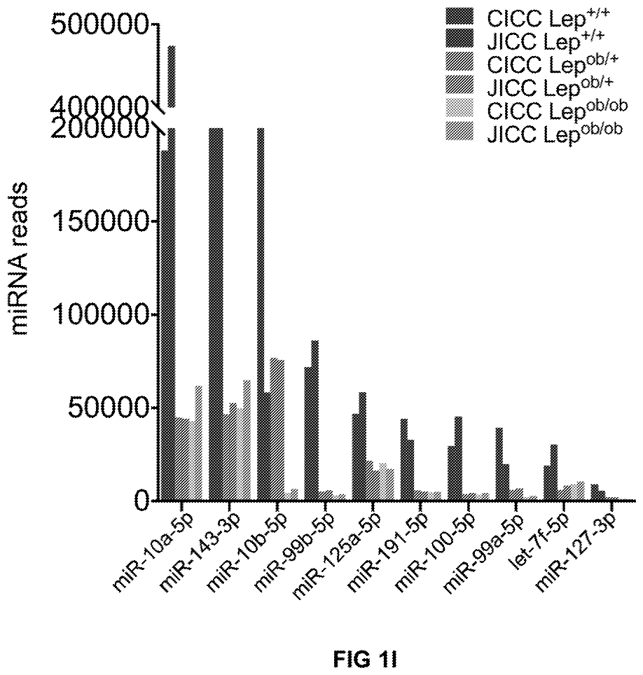

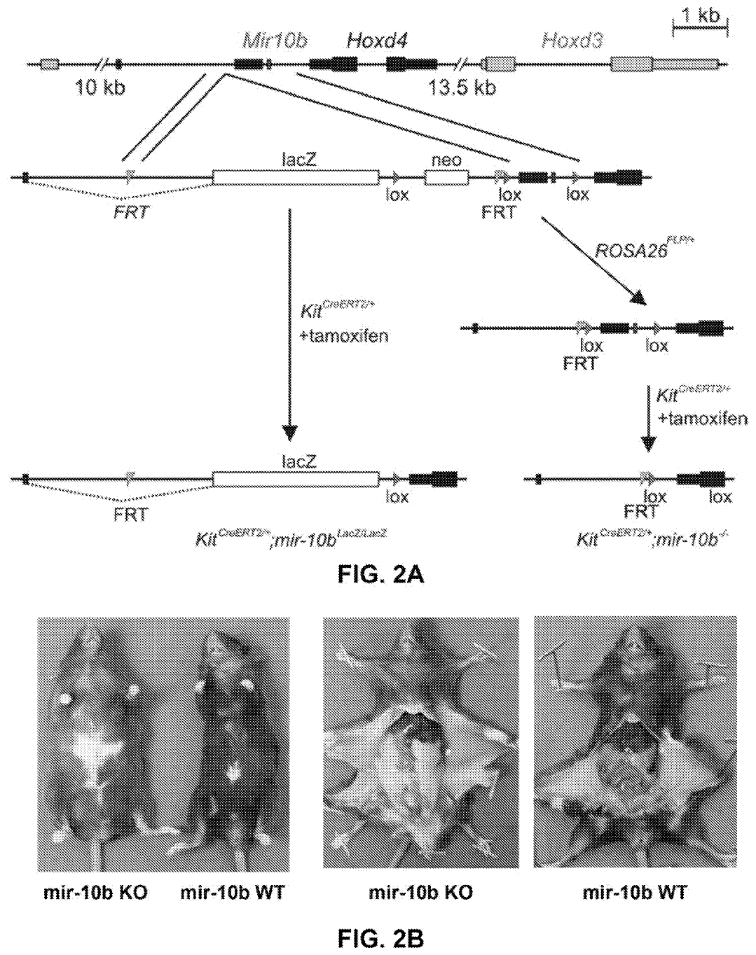

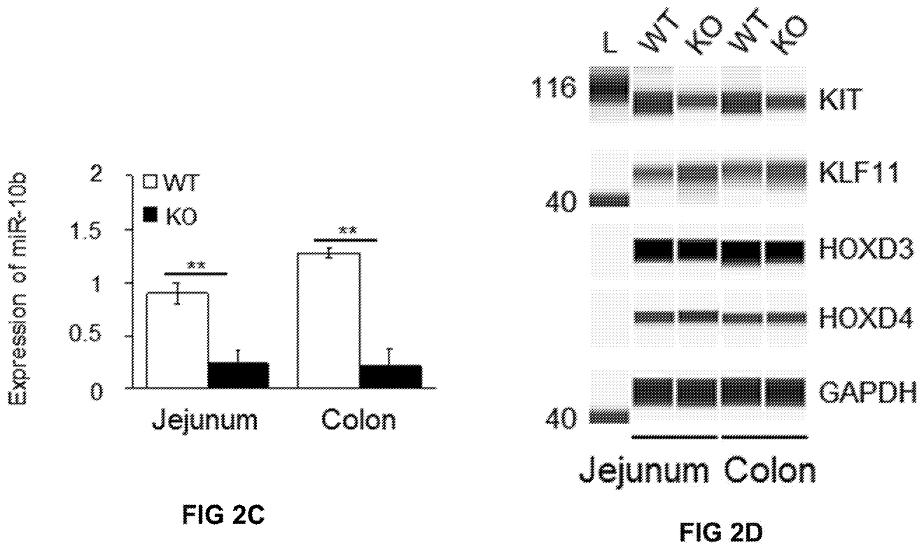

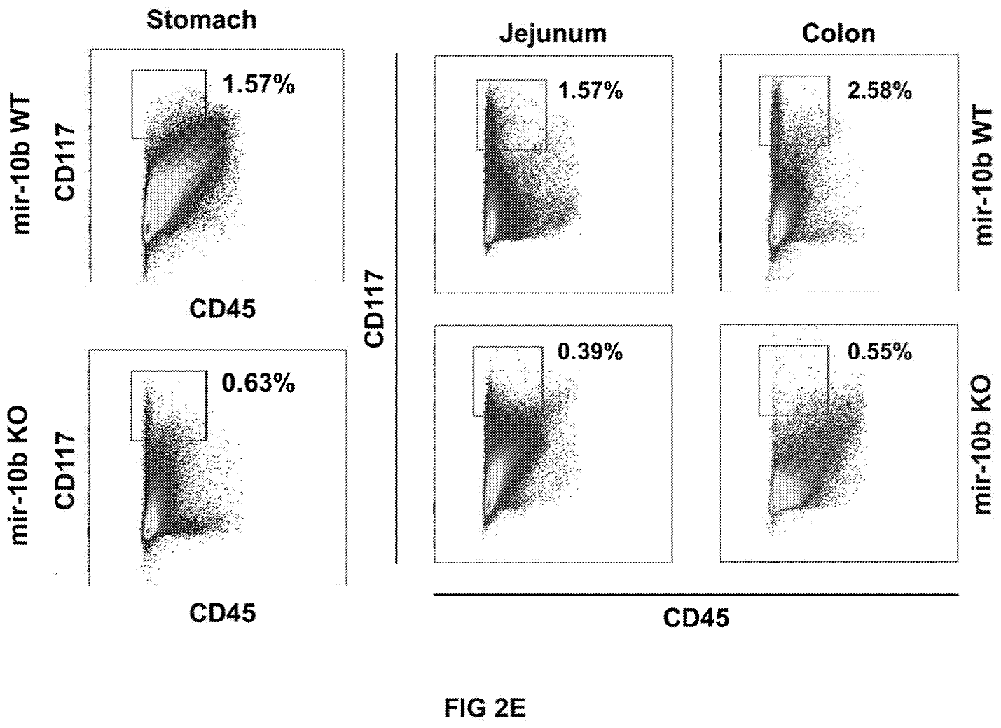

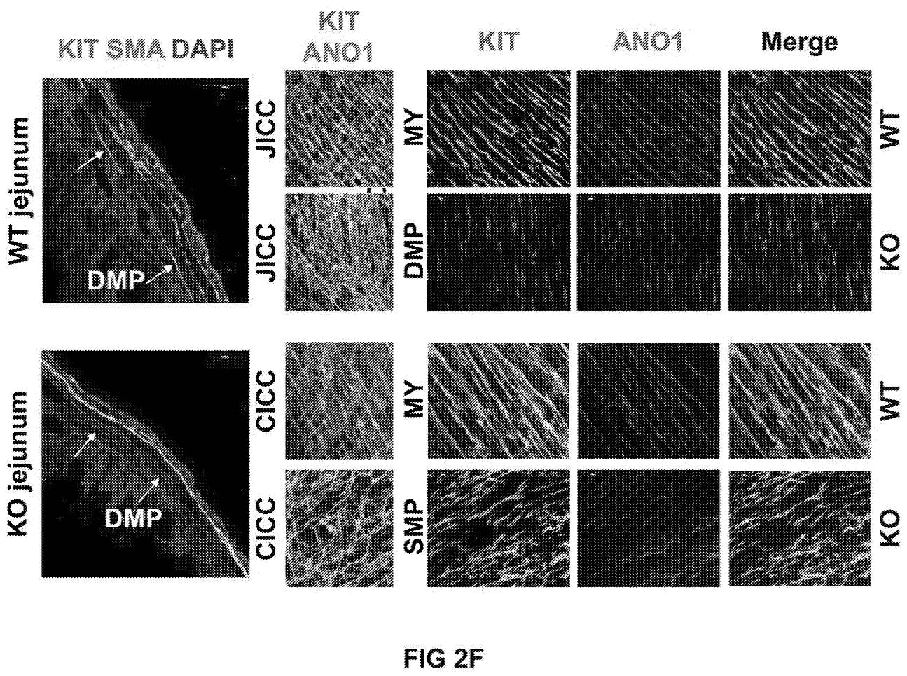

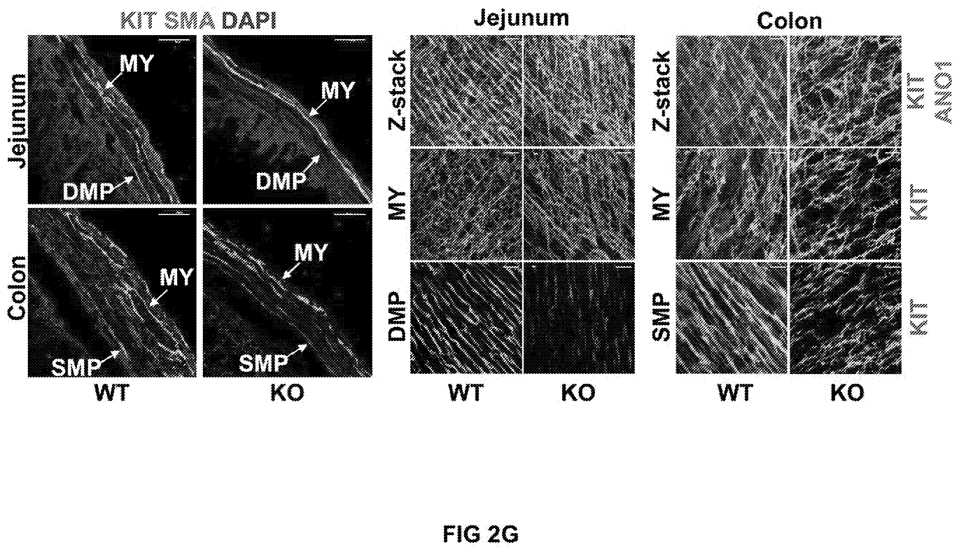

The Mir10b gene is located within the fifth intron of Hoxd4, which is also overlaid with the intron 1 of Hoxd3 on chromosome 2 (FIG. 2A). A foxed Mir10b litter (mir-10b^{LacZ-FLP-lox/+}) was recovered from cryo-archive (The Jackson Laboratory). Mir-10b^{LacZ-FLP-lox/+} mice were crossed with Kit^{CreERT2/+} mice or ROSA26^{FLP/+} mice to generate Kit^{CreERT2/+}; mir-10b^{LacZ/LacZ} mice and ROSA26^{FLP/+};mir-10b^{lox/lox} mice (FIG. 2A). ROSA26^{FLP/+}; mir-10b^{lox/lox} mice were further crossed with Kit^{CreERT2/+} mice to generate Kit^{CreERT2/+};mir-10b^{lox/lox} mice. Kit^{CreERT2/+};mir-10b^{LacZ/LacZ} mice and Kit^{CreERT2/+};mir-10b^{lox/lox} mice were treated with tamoxifen to delete mir10b in ICC. Both lines injected with tamoxifen for 5 consecutive days resulted in mir-10b KO (Kit^{CreERT2/+};mir-10b^{-/-}). Male mir-10b KO and WT mice at 6 months after tamoxifen and oil injection are shown in FIG. 2B. Mir-10b KO mice are heavier and contained more abdominal fat than WT controls. Mir-10b deletion in mir-10b KO mice was confirmed through qPCR (FIG. 2C). Expression of miR-10b-5p was significantly decreased in mir-10b KO jejunal and colonic smooth muscle. In the KO jejunal and colonic smooth muscle, expression of KIT protein was also decreased (FIG. 2D). MiR-10b-5p is predicted to target KLF11 in TargetScan. KLF11 was increased in KO tissues, suggesting that miR-10b-5p targets KLF11. In addition, expression of HOXD3 and HOXD4 was not changed in the KO tissues, suggesting that deletion of Mir10b, encoded in the intronic region of the two overlapping genes, did not disrupt the expression of these two genes. ICC, analyzed through CD117⁺ (KIT antibody) and CD45⁻ (hematopoietic cell marker) selection in flow cytometry, were significantly decreased in the mir-10b KO small intestine and colon (FIG. 2E). Loss of ICC in the jejunum and colon was confirmed by immunohistochemistry (FIG. 2F). ICC subtypes, ICC DMP in jejunum and ICC SMP in colon, appeared to be reduced most in the KO tissues. The results are shown in FIGS. 2A-2F.

### Example 3—Male Mir-10b Knockout Mice Develop Diabetes

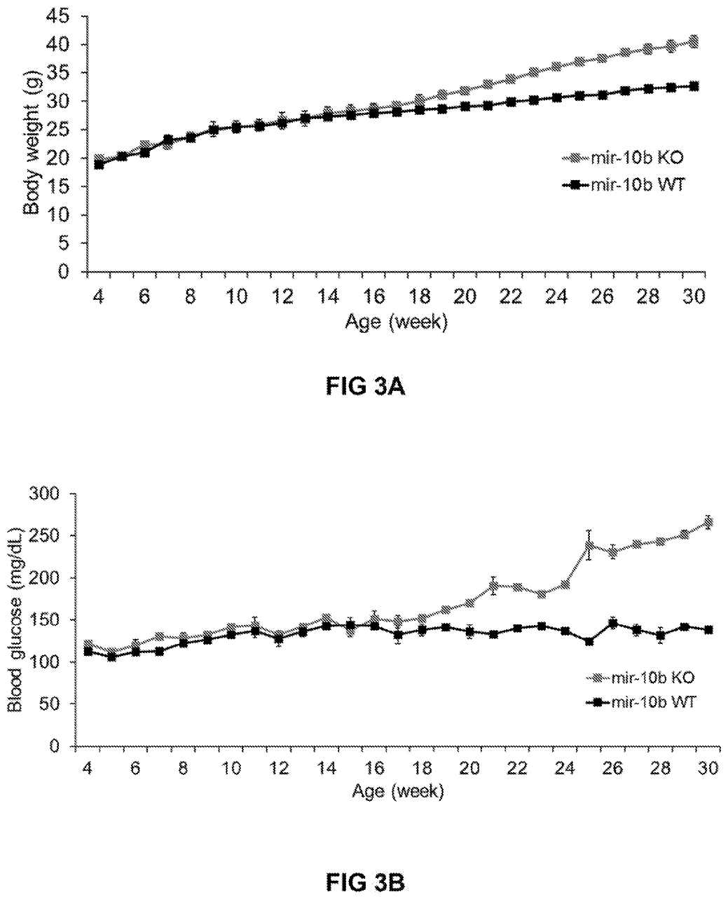

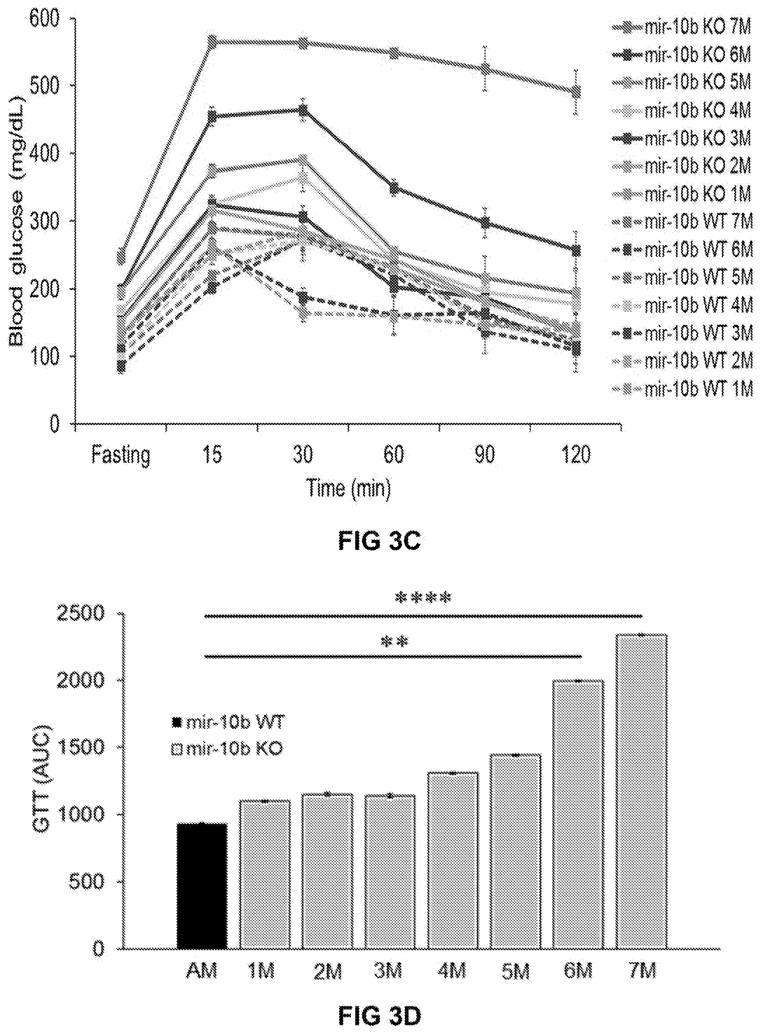

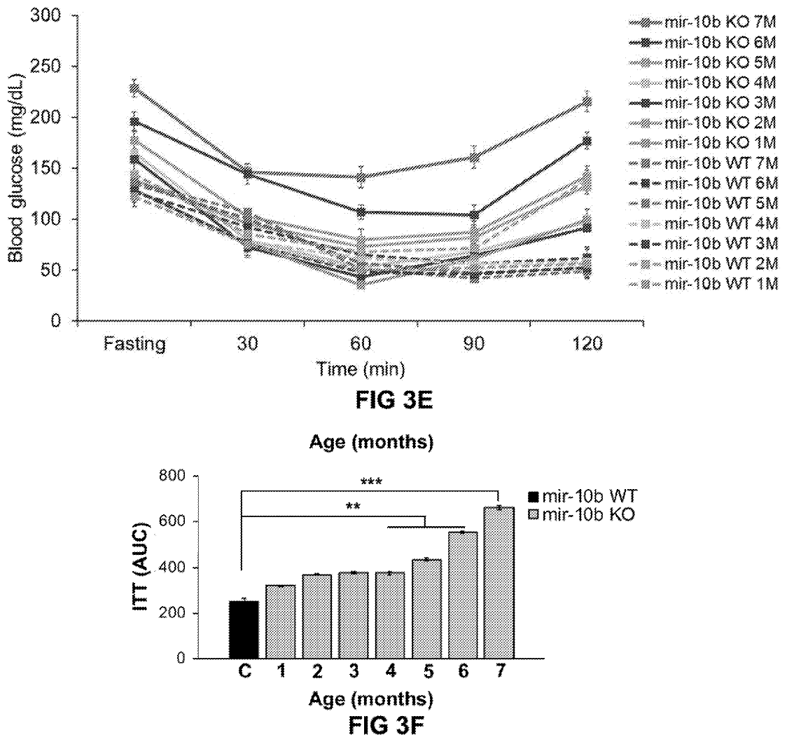

Male mir-10b KO mice, but not female mice, became moderately obese and developed type 2 diabetes (FIGS. 3A-3F). Body weight of mir-10b KO male mice progressively increased after 16 weeks (4 months) post-tamoxifen injection and was significantly heavier (40.5 g vs 32.7 g) at 30 weeks (6 and half months), compared to control male mir-10b WT mice (FIG. 3A). Blood glucose levels of male mir-10b KO mice also progressively increased and the mice became hyperglycemic (over 200 mg/dL) after 24 weeks (6 months) (FIG. 3B). Intraperitoneal glucose tolerance test (GTT) in mir-10b KO mice confirmed that they progressively impaired their response to a glucose challenge from 1 month to 7 months although 1-7 months old WT control mice could clear the challenged glucose within 2 hours (FIG. 3C). There is significant increase of glucose intolerance in mir-10b KO mice at 6-7 months (FIG. 3D). In addition, mir-10b KO mice progressively impaired insulin sensitivity from 1 month to 7 months (FIG. 3E). There is significant increase of insulin resistance in mir-10b KO mice after 4 months, compared to mir-10b WT mice (FIG. 3F). The results are shown in FIGS. 3A-3F.

### Example 4—Male Mir-10b Knockout Mice Display Prolonged Gastrointestinal (GI) Transit

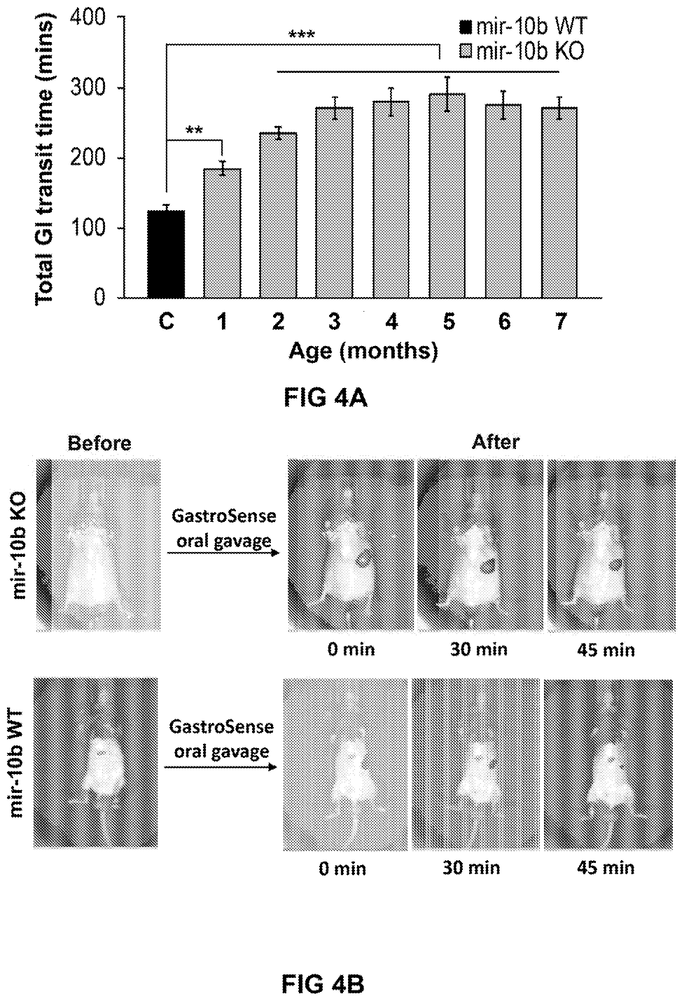

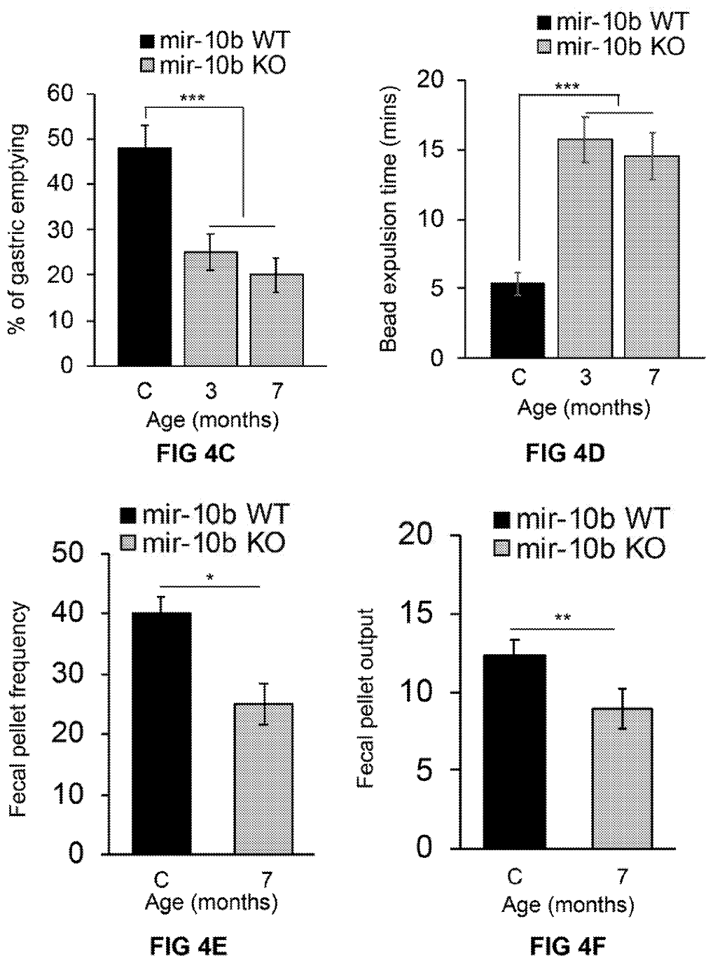

Mir-10b KO mice also develop gastroparesis and constipation. The KO mice show prolonged total GI transit starting early at 1 month old, which was progressively delayed until 3 months of age and stabilized to be constipated after that (FIG. 4A). At 3 months old, transit time was delayed over two times (125 min vs 270 min). The delayed total GI transit resulted from slow gastric emptying and slow colonic transit. The IVIS in vivo imaging showed that gastric emptying in mir-10b KO mice at 7 months old was obviously delayed compared to WT controls (FIG. 4B). The gastric emptying time in KO mice was almost two times slower at 3 months of age and further slowed at 7 months old (FIG. 4C). Colonic transit in KO mice was also further delayed by over three times (FIG. 4D). Moreover, the average fecal pellet frequency and output in the KO mice was significantly lowered (FIGS. 4E-4F). All these data consistently suggest that the mir-10b KO mice exhibited severely reduced GI motility. More importantly, the motility disorder precedes impaired glucose and insulin tolerance in the mir-10b KO mice. The results are shown in FIGS. 4A-4F.

### Example 5—Pathway Analysis and Target Validation for miR-10b-5p in Diabetes

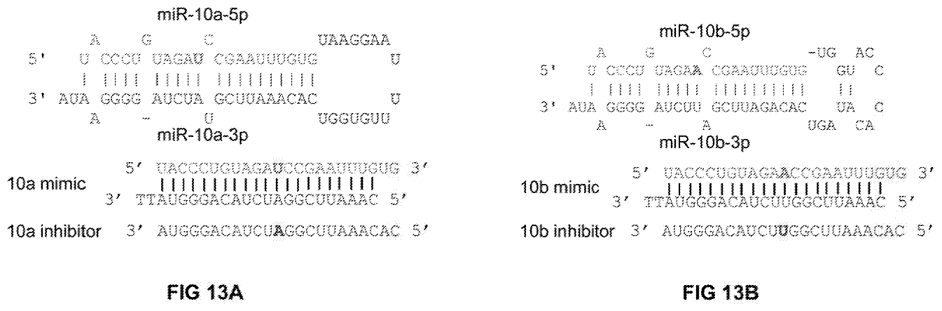

MiR-10b-5p and its target genes associated with diabetes were analyzed by the Ingenuity Pathway Analysis (IPA) software. This analysis identified many miR-10b-5p targets including KLF4, KLF7, and KLF11, which are linked to diabetes (FIG. 5A). KLF11 is a transcriptional repressor which was selected for further studies based on interactions with INS. KLF11 binds both GC boxes and CACCC boxes in the insulin (INS) promoter (Niu 2007; Neve 2005), and suppresses transcriptional activation of the target gene (Niu 2007). The interacting genes (INSR, IRS1, IRS2, KIT, NEUROG3, SLC2A4, and GLP1R) contain a GC-rich CpG island, suggesting that these genes are potential KLF11 targets. Two mature miRNAs, miR-10b-5p and -3p are generated from a precursor (pre-miR-10b), which is highly conserved in both mice and humans: the mature miRNAs are the same (Kozomara 2019). For miRNA-target screening, six chemically synthesized forms of miR-10b-5p mimic (10b mimic 1, duplex RNA from Thermo Fisher Scientific; 2, duplex RNA from Dharmacon; 3, duplex RNA from QIAGEN; 4, single stranded miR-10b-5p RNA from IDT; 5, annealed miR-10b-3p and miR-10b-5p RNA from IDT; 6, precursor miR-10b RNA from IDT) were tested in murine NIT3T3 cells and human HEK 293 along with antisense miR-10b-5p (single stranded 10b inhibitor from Thermo Fisher Scientific; single stranded miR-10b-3p from IDT), two mouse Klf11 (siKlf11-1, siKlf11-2) and human KLF11 siRNAs (siKLF11-1, and siKLF11-2) (Thermo Fisher Scientific). Mouse pre-miR-10b encoding miR-10b-5p and -3p, 10b mimic and inhibitor are shown in FIG. 13B. Effects on the target genes in NIH3T3 and HEK293 cells transfected with the 10b mimic and inhibitor is shown in FIG. 5C. Western blots show that miR-10b-5p efficiently targets Klf11 in transfected NIH3T3 and HEK293 cells (FIG. 5C). The duplex RNA miR-10b mimic 1 most efficiently suppressed the translation of the KLF11 protein, followed by pre-miR-10b. Two other miR-10b mimics (2 and 3), single stranded miR-10b-5p RNA, and annealed miR-10b-3p and miR-10b-5p RNA suppressed the protein much less than the mimic 1 or did not incite any suppression. On the other hand, the inhibitor miR-10b-3p RNA drastically increased KLF11, suggesting the inhibitor efficiently binds and knocks down endogenous miR-10b-5p levels. In addition, KIT expression was negatively regulated by the amount of concurrent KLF11, suggesting KLF11 represses KIT expression in NIH3T3 and HEK293 cells. miR-10b mimic 1 was selected and used for further targeting experiments. Diabetic genes suppressed by KLF11 were assessed in four mouse and human cell lines (NIH3T3; HEK293; mouse β cell, NIT-1; human β cell Panc 10.05) using 10b mimic, inhibitor, two mouse and human siKLF11s. KLF11 negatively regulated KIT, IRS1 and IRS2 in NIH3T3 and HEK293 (FIG. 5D) and KIT, IRS1, IRS2, INS, and ISNR in the mouse and human β cell lines (FIG. 5E). KLF11 expression was decreased both by the 10b mimic and two siKLF11-1 and -2. KLF11 levels were increased upon introduction of the 10b inhibitor in all four cell lines, confirming the regulatory pathway of the diabetic genes by miR-10b-5p (mimic)-KLF11. For further target validation of 10b mimic-KLF11, a miR-10b-5p target site in mouse Klf11 (mKlf11 10b TS) and human KLF11 (hKLF11 10b TS) 3′UTR was inserted at the end of a luciferase gene in reporter gene plasmids (FIG. 5F). The two reporter gene plasmids containing mKlf11 10b TS mutant (mKlf11 10b TSM: 4 mutations in the seed sequence) and scramble sequences were also constructed and used as negative controls (FIG. 5F). The mouse and human KLF11 10b TS sequence are highly conserved (FIG. 5F). Four stable cell lines (NIH3T3, HEK293, mouse and human β cell lines) transformed with each of the reporter gene plasmids were generated. NIH3T3-mKlf11 10b TS transfected with six series of 10b mimic and 10b inhibitor (1-200 pmol) showed dose-dependent reduction and induction of luciferase activity at 6, 12, 24 and 48 hours, but most reduction and induction at 12 hours (FIG. 5G). There was no reduction or induction of luciferase activity in NIH3T3-mKlf11 10b TSM transfected with 10b mimic/inhibitor (FIG. 5G). Then the four cell lines with mKlf11/hKLF11 10b TS, mKlf11/hKLF11 10b TSM, and/or scramble were transfected with 10b mimic and inhibitor in a series of various concentrations. All four cell lines with mKlf11/hKLF11 10b TS showed dose-dependent inhibition of luciferase by 10b mimic and induction of the enzyme by 10b inhibitor (FIG. 5H). But the cells with mKlf11/hKLF11 10b TSM and/or scramble had no significant effect by 10b mimic and inhibitor. Taken together, these in vitro experiments demonstrated that miR-10b-5p negatively regulates both mouse and human KLF11 which, in turn, suppresses expression of the diabetic genes. The results are shown in FIGS. 5A-5H.

### Example 6—Rescue of Diabetes and Gastroparesis in Mir-10b Knockout Male Mice by miR-10b Mimic Injection

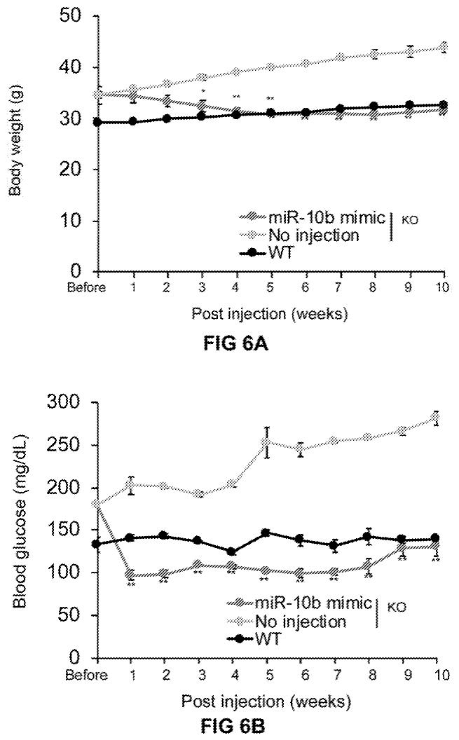

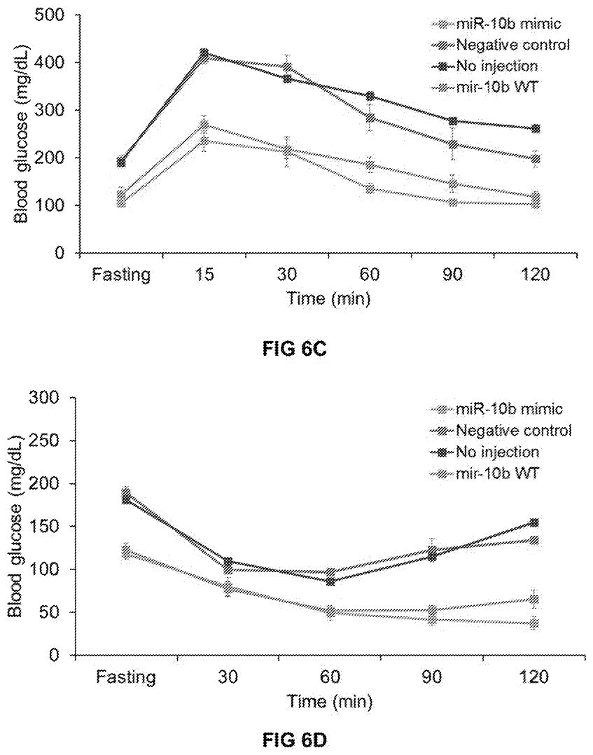

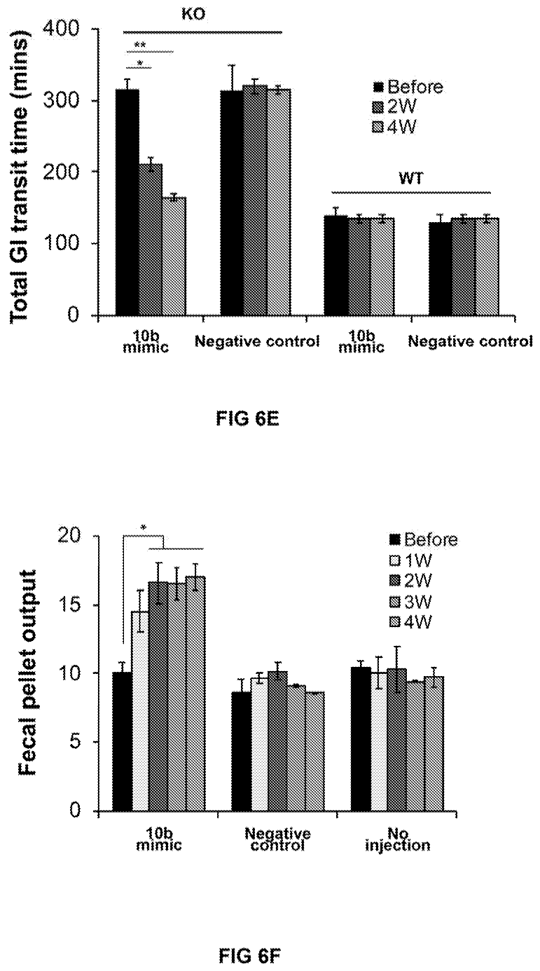

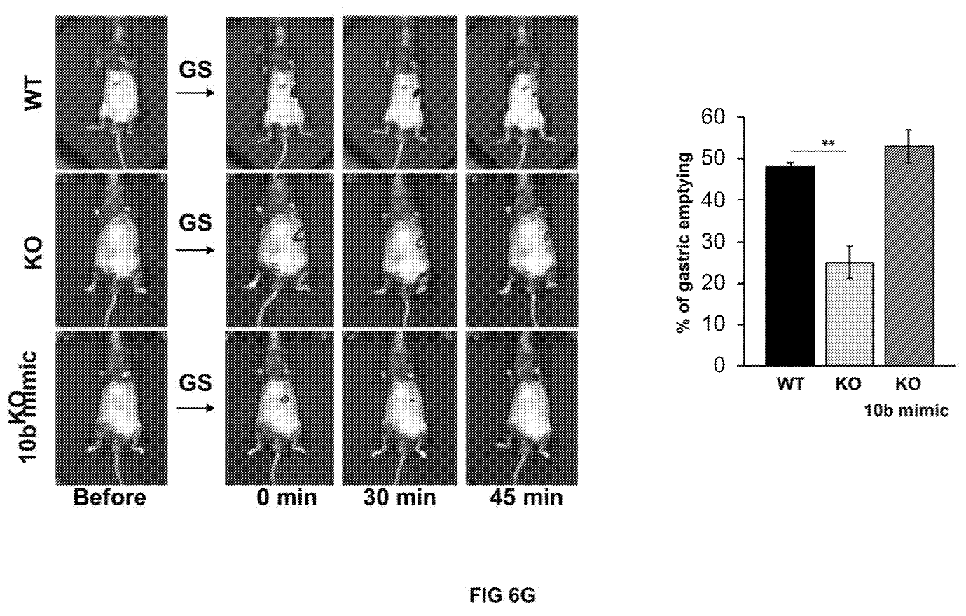

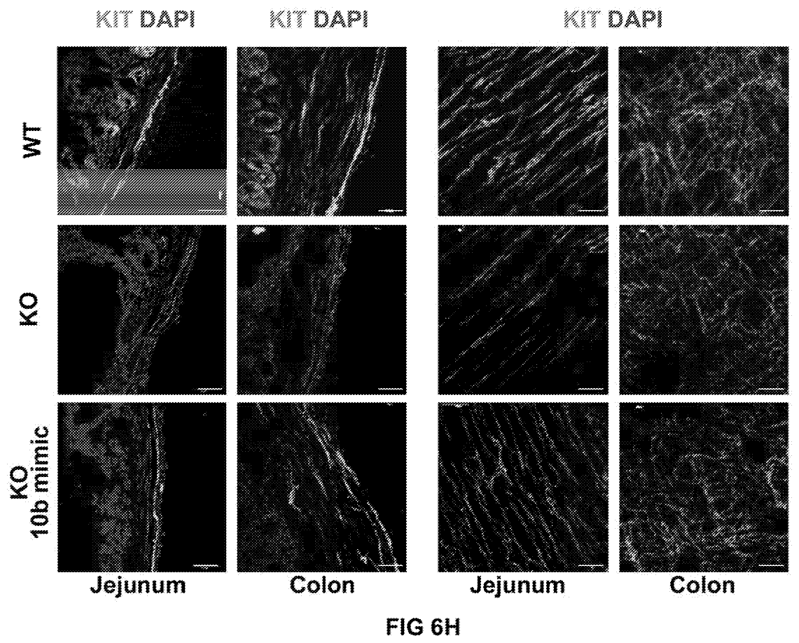

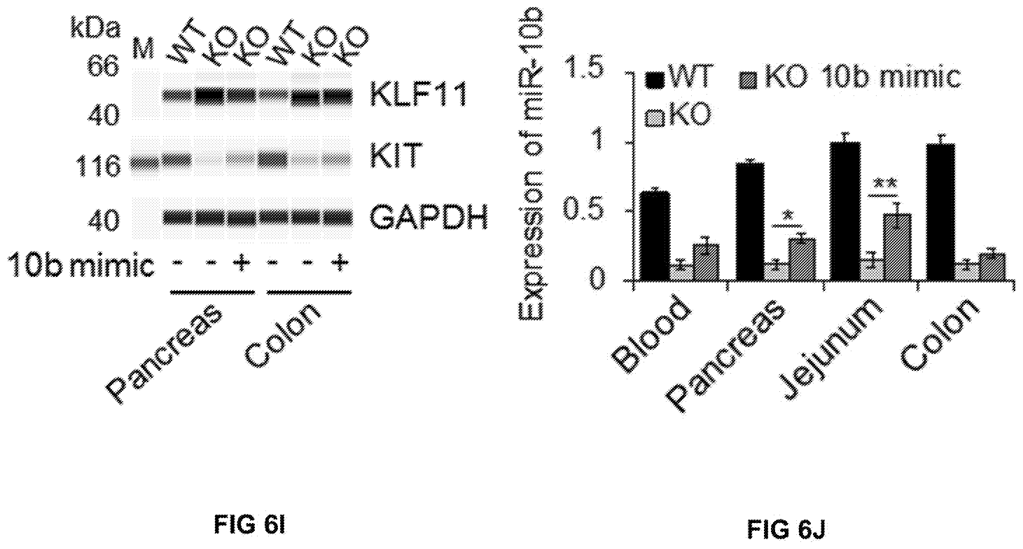

To test the in vivo effect of 10b mimic, 10b mimic (263 ng/g) was delivered with in vivo jetPEI into mir-10b KO diabetic mice by intraperitoneal injection. Body weight in three groups (10b mimic injection and no injection in mir-10 KO and WT mice) were compared for 10 weeks after injection. The 10b mimic injected mice lost small amounts of weight over 4 weeks while none injected KO mice gradually gained weight (FIG. 6A). More importantly, the 10b mimic injected mice immediately lowered their fasting glucose levels to normal levels (˜100 mg/dL) at 1 week and maintained it up to 10 weeks (FIG. 6B). GTT and ITT showed that the mimic injected mice improved glucose tolerance and insulin resistance similar to or even slightly better than WT mice (FIGS. 6C-6D). Furthermore, GI function in the mimic injected mice was gradually improved and restored. The fecal output was gradually increased at 1 and 2 weeks where it became stabilized after 2 weeks (FIG. 6E). Total GI transit time was also decreased at 2 and 4 weeks, which then became similar to WT mice (FIG. 6F). Furthermore, gastric emptying time was greatly improved and entirely restored in the 10b KO diabetic mice injected by 10b mimic to be similar to WT mice (FIG. 6G). ICC in jejunum and colon were lost in the 10b KO mice, but they were restored in mir-10 KO mice after miR-10b-5p mimic injection (FIG. 6H). Restoration of KIT protein expression was also confirmed in pancreas and colon in the 10b KO mice. Furthermore, increased miR-10b-5p after injection of 10b mimic was confirmed in blood, pancreas, jejunum, and colon (FIG. 6J). The results are shown in FIGS. 6A-6J.

### Example 7—miR-10b-5p Mimic Rescues Diabetes and GI Slow Transit in High Fat and High Sucrose Diet (HFHSD) Mice

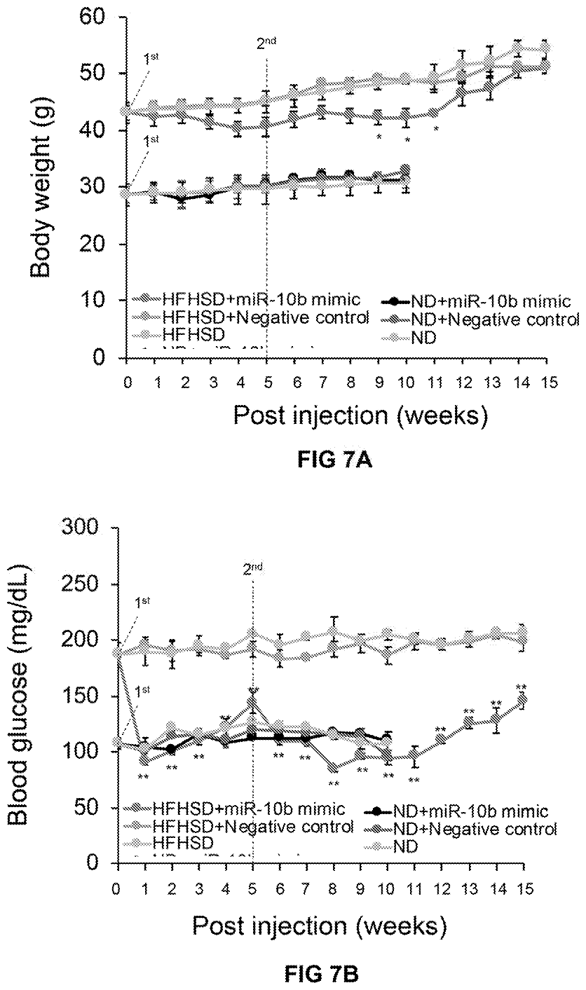

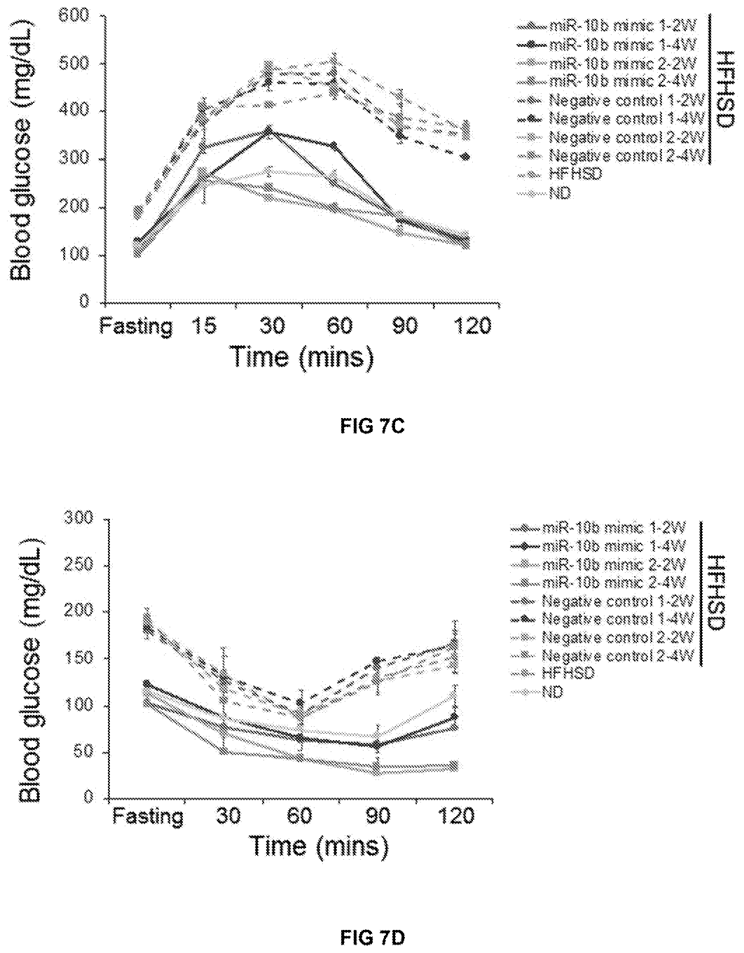

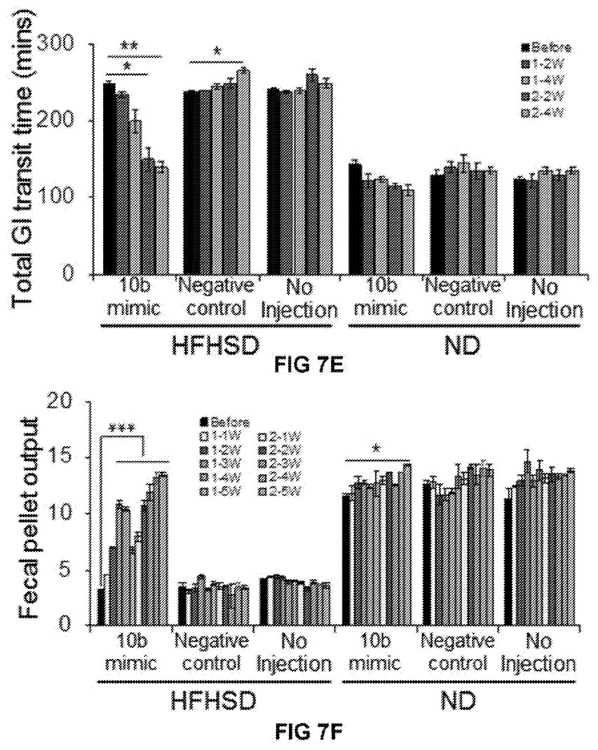

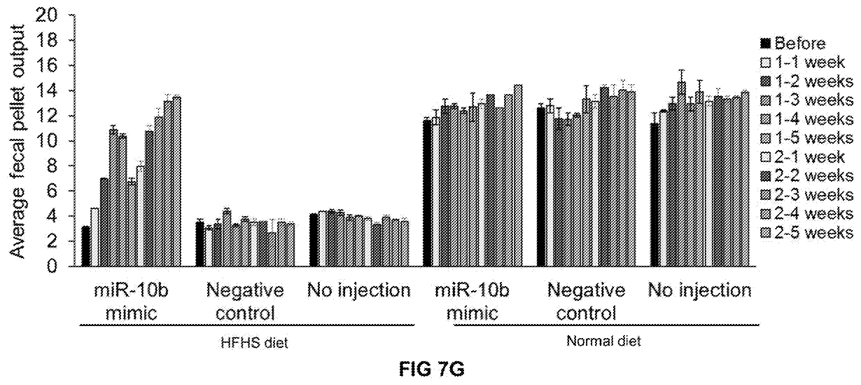

Next, an experiment was designed to test whether the 10b mimic can rescue HFHSD induced diabetic mice. Kit^{copGFP/+} mice were fed with either a HFHS diet or a normal diet for 4 months. HFHS diet mice (˜43 g) significantly gained more weight and increased blood glucose (˜200 mg/dL), compared to normal diet mice (29 g and ˜107 mg/dL). Both groups of mice were injected intraperitoneally twice (1^{st} injection followed by 2^{nd} injection with 5-week interval) with 10b mimic (500 ng/g) and negative control RNA (500 ng/g) with in vivo jetPEI, or not injected. Body weight, blood glucose levels, and GI function in the three groups (10b mimic injection, negative control RNA injection, and no injection) in HFHSD mice and normal diet mice were compared for 15 weeks after the two injections. The 1^{st} and 2^{nd} 10 b mimic injected mice in HFHSD mice did not gain any weight over 11 weeks while the negative control and none injected mice fed the HFHSD mice gradually gained weight (FIG. 7A). The three groups in normal diet mice also gradually gained weight, but much slower than HFHSD fed mice. The 10b mimic injected HFHS diet mice have immediately and remarkably lowered fasting glucose levels to normal levels (90 mg/dL) at 1 week as seen in 10b mimic injected 10b KO mice (FIG. 7B). However, the glucose level progressively bounced back and reached pre-diabetic levels (150 mg/dL) at 5 weeks. The glucose level dropped to normal levels after the 2^{nd} injection and maintained at 100-140 mg/dL for 10 weeks. The three groups in normal diet mice maintained glucose at normal levels. GTT and ITT showed that the mimic injected HFHSD mice had improved glucose tolerance and insulin resistance similar to, or better than, normal diet mice (especially after the 2^{nd} injection) (FIGS. 7C-7D). In addition, GI function in the mimic injected HFHSD progressively improved and was restored at 2 and 4 weeks in 1^{st} and 2^{nd} injection (FIG. 7E). GI transit time at 4 weeks after 2^{nd} injection was almost similar to that of normal diet mice. The average pellet frequency and fecal output gradually increased from 1-4 weeks and decreased at 5 weeks in the 1^{st} mimic injected HFHSD mice (FIG. 7F). However, the 2^{nd} injection led to a much improved frequency and output at 5 weeks, similar to normal diet mice. The results are shown in FIGS. 7A-7F.

### Example 8—Restoration of miR-10b, its Regulated Proteins, ICC, and β Cells in Diabetic Mice Treated with 10b Mimic

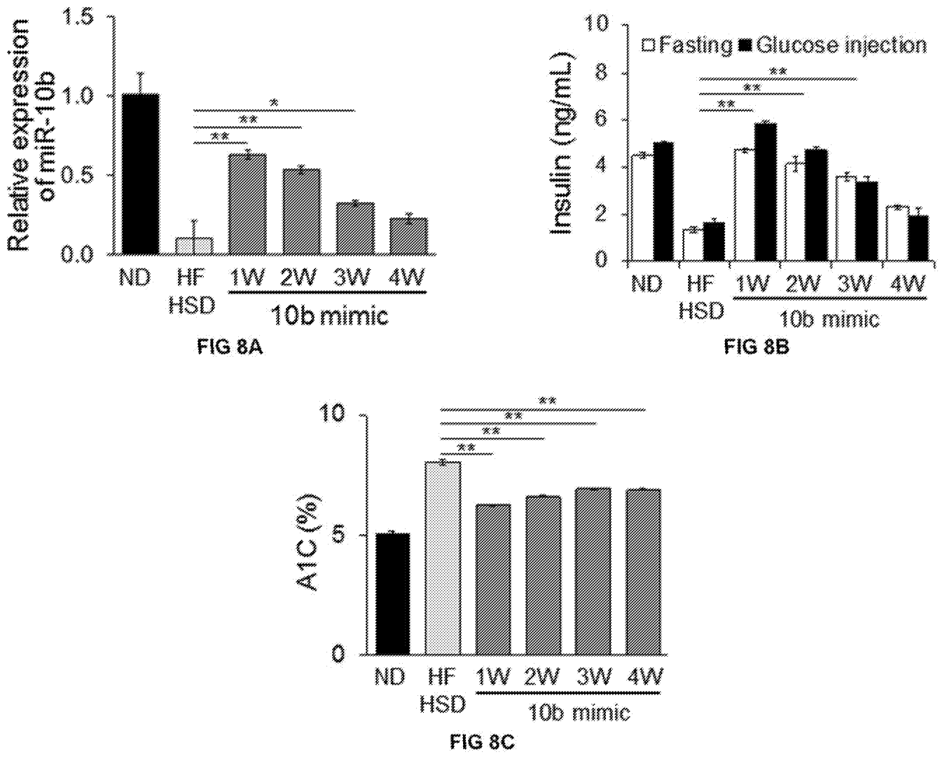

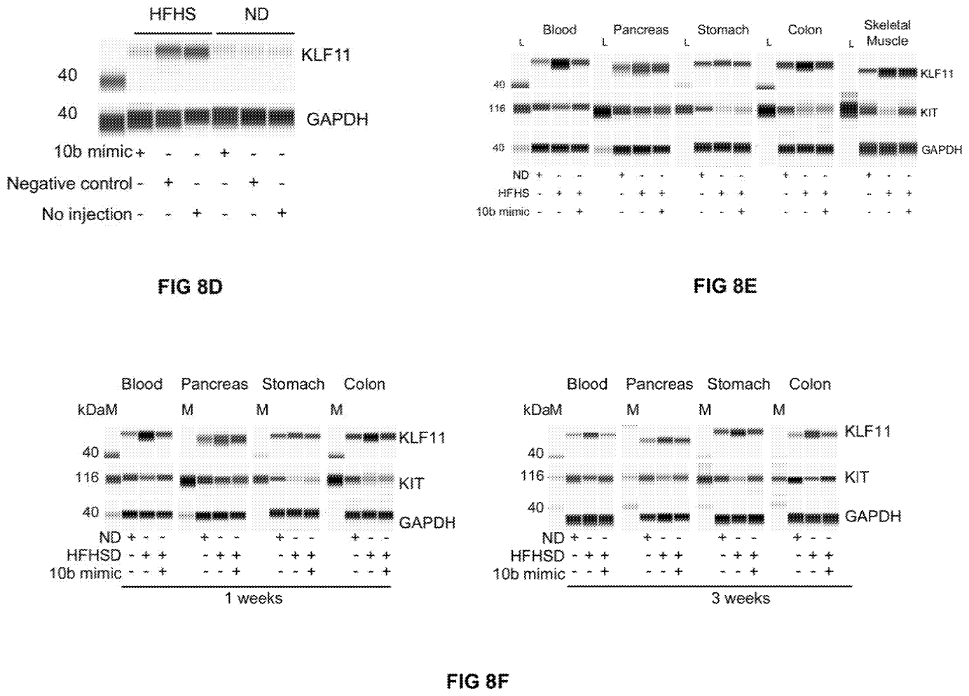

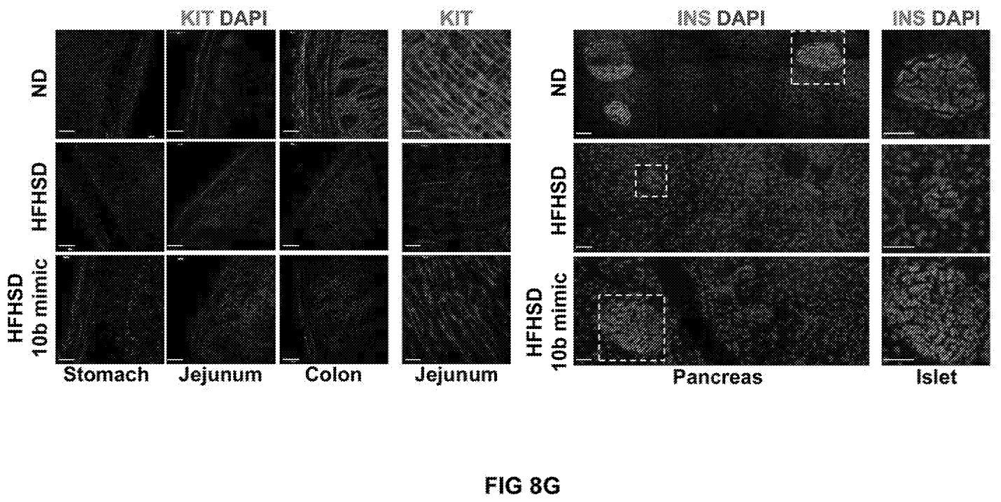

Reduction of miR-10b-5p in the blood of HFHSD diabetic mice was confirmed by qPCR (FIG. 8A). The miRNA was dramatically reduced in diabetic mice compared to normal diet healthy controls. The miRNA was restored by around 63% through 10b mimic injection at 1 week and gradually declined at 2-4 weeks. Surprisingly, the levels of the miRNA were paired with those of insulin in blood in mice after 6 hours of fasting and subsequent glucose challenge (FIG. 8B). In addition, insulin was higher in 10b mimic injected mice at 1 week than healthy control mice. Percent of hemoglobin A1C was the reverse of insulin and C-peptide data (FIG. 8C). 10b mimic injection decreased A1C % which slowly rebounded at 1-4 weeks. KLF11 protein changes were examined in the blood, pancreas, stomach, and colon of 10b mimic injected mice through Western blotting (FIG. 8D). KLF11 was highly increased in none injected mice fed a HFHSD while it was much expressed at much lower levels in normal diet fed mice. 10b mimic injection into HFHSD mice efficiently depleted KLF11 in the tissues at 1 and 3 weeks post injection (FIG. 8D). In addition, KIT was decreased in the tissues in HFHSD, and increased by 10b mimic at the first week and further increased at the third week (FIG. 8D). Moreover, ICC were lost in HFHSD, but readily detected in 10b mimic injected stomach, jejunum, colon and pancreas (FIG. 8E). The results are shown in FIGS. 8A-8G.

### Example 9—Preventive Effects of miR-10b-5p Mimic Against Diabetes and Slow GI Transit in HFHSD Mice

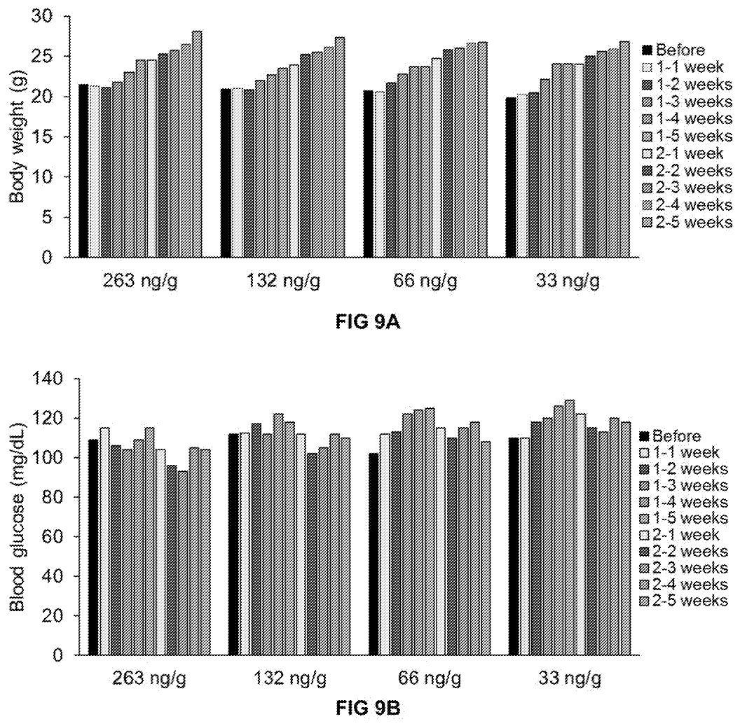

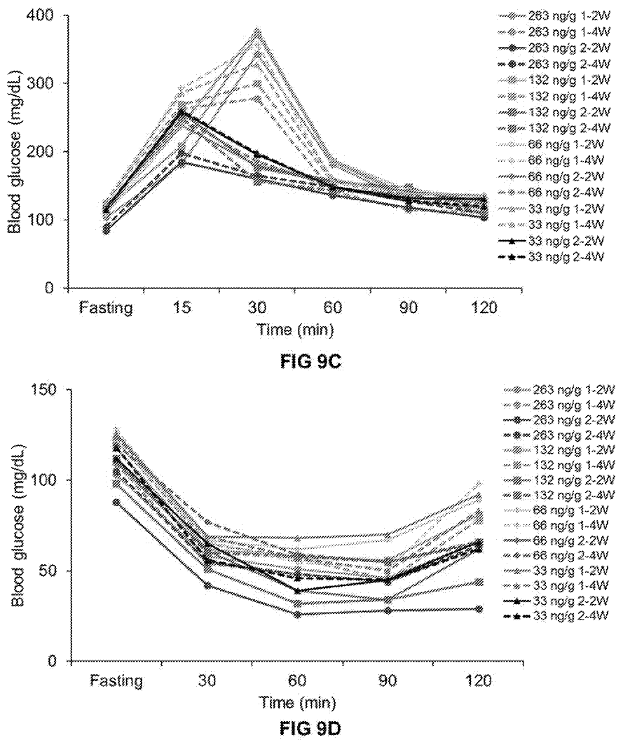

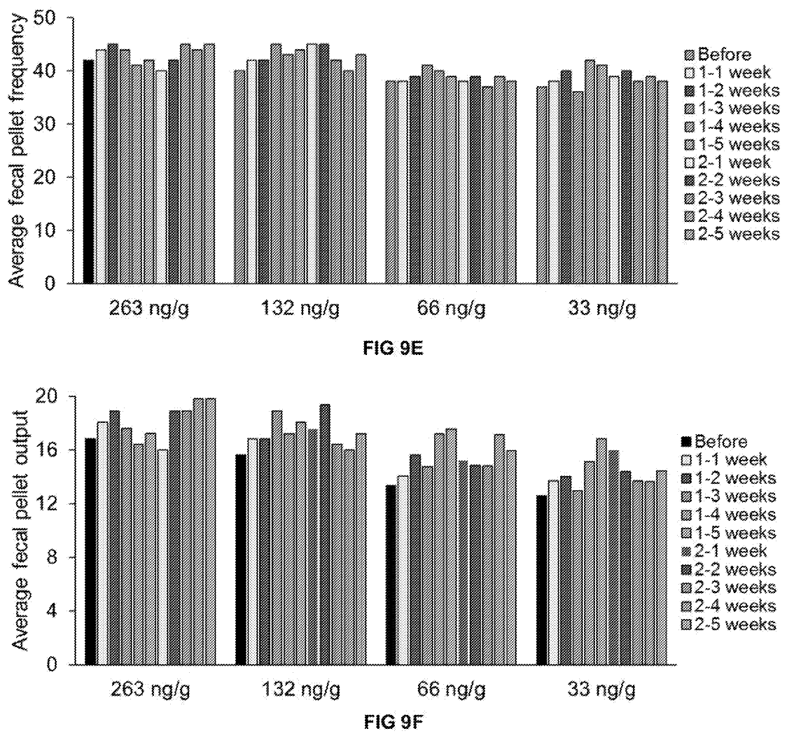

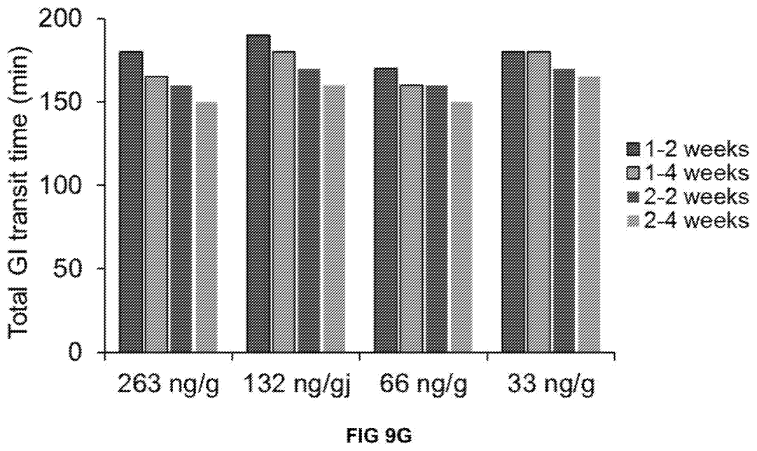

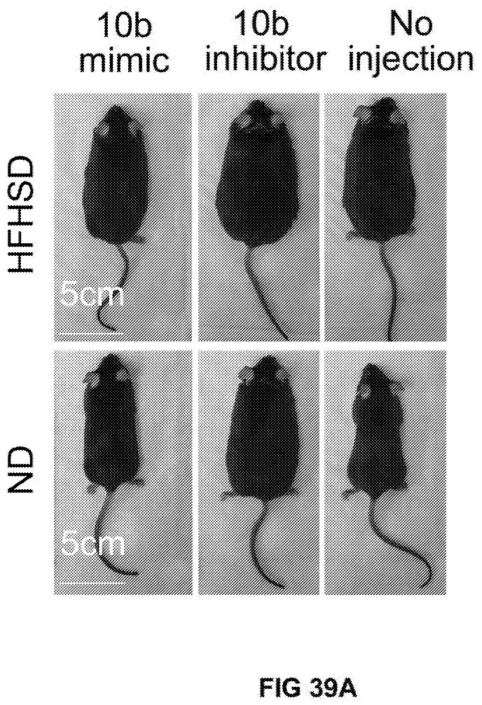

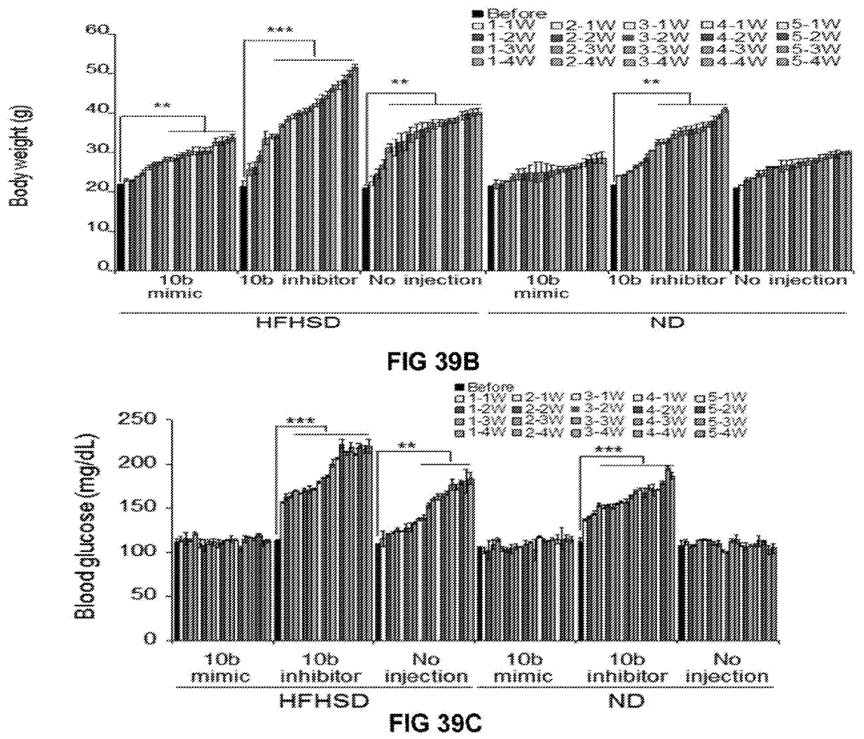

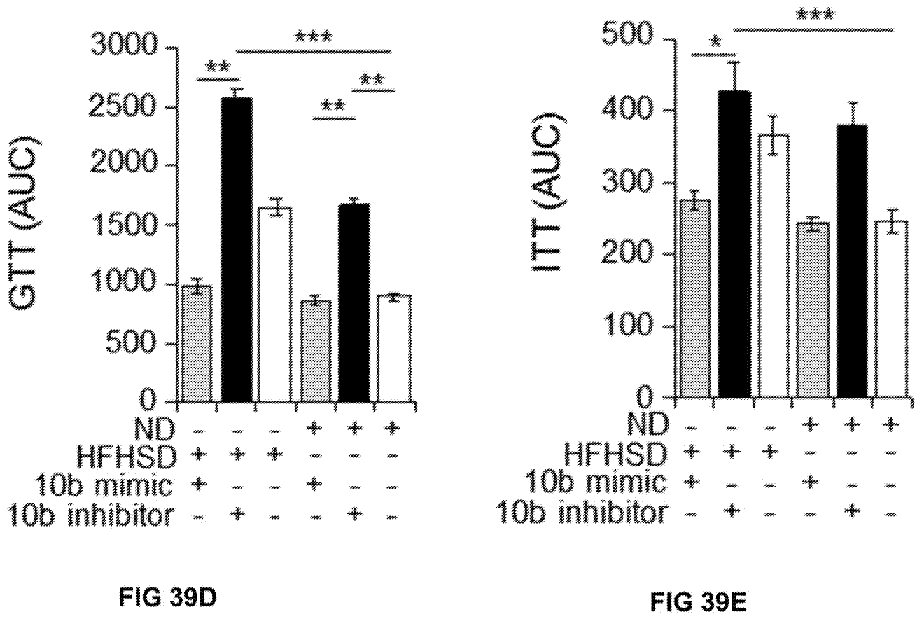

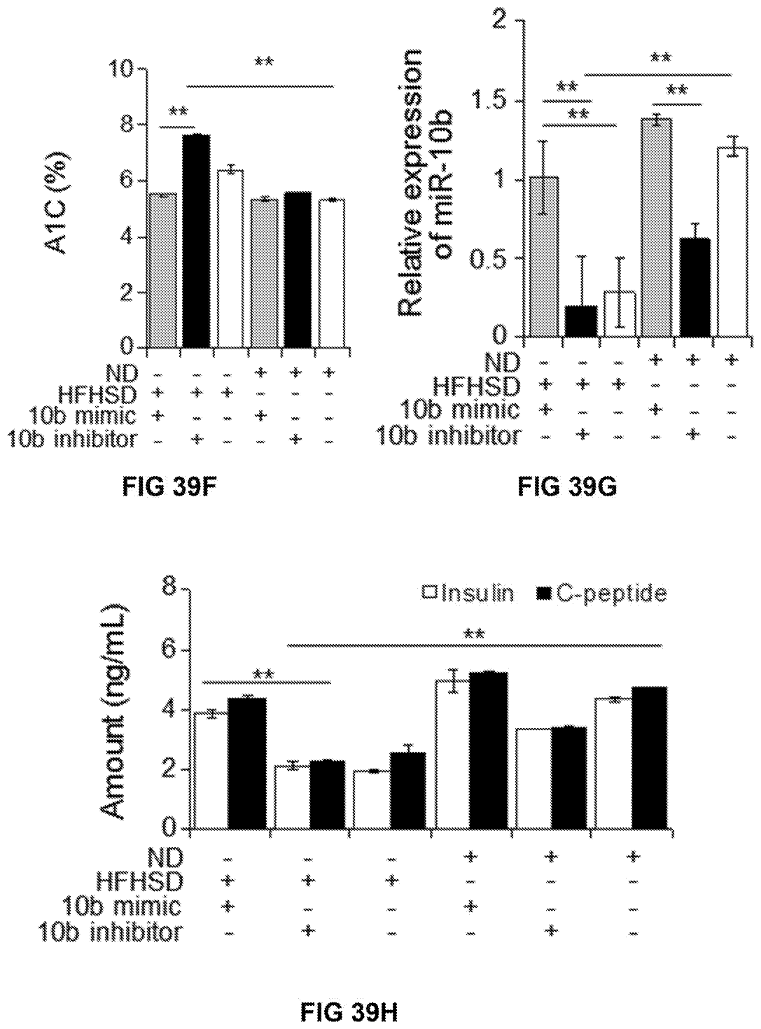

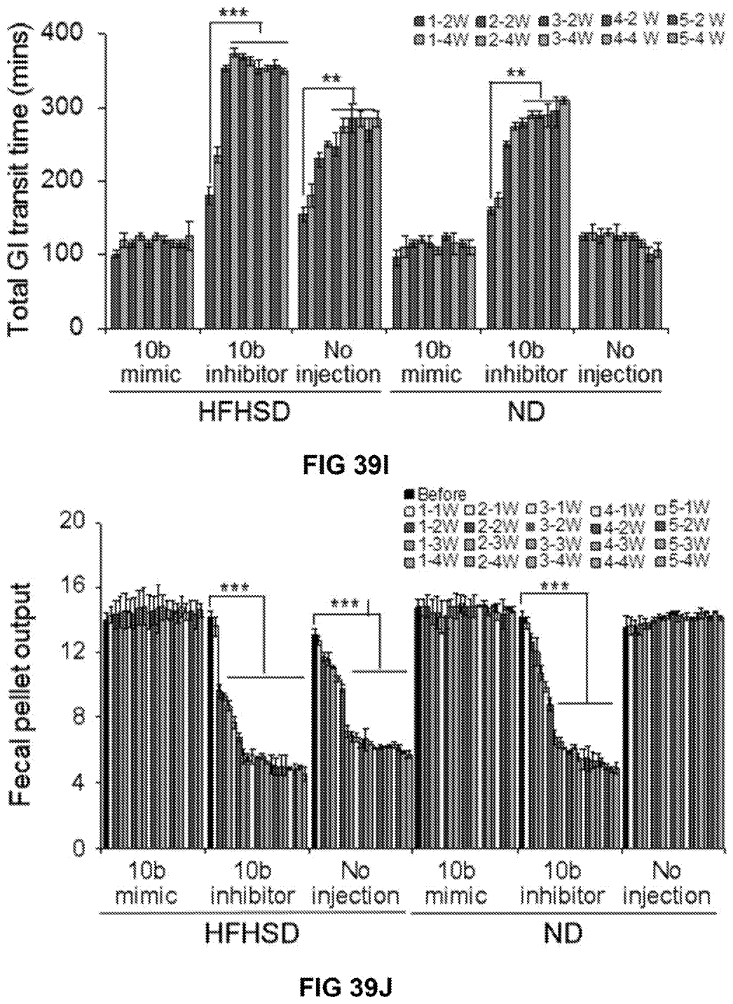

To determine whether miR-10b-5p can prevent the onset of diabetes and gastroparesis in mice fed a HFHSD, the miR-10b-5p mimic or miR-10b-5p inhibitor was injected monthly into healthy C57 mice fed either a HFHSD or a ND. Non-injected control mice fed a HFHSD rapidly gained weight, whereas ND-fed control mice gained weight at a slower rate. (FIGS. 39A and 39B) The HFHSD-fed mice injected with the miR-10b-5p mimic gained weight slowly over 5 months, similar to ND-fed control mice, while miR-10b-5p inhibitor-injected mice gained weight rapidly in both the HFHSD and ND-fed groups. (FIGS. 39C-39E) Blood glucose levels substantially increased in miR-10b-5p inhibitor-injected mice and reached diabetic levels (>180 mg/dL) in HFHSD-fed mice and pre-diabetic levels (121-179 mg/dL) in ND-fed mice, whereas miR-10b-5p mimic-injected mice maintained healthy blood glucose levels over a 5 month time period. (FIG. 39G) Levels of miR-10b-5p in the blood were restored and maintained through monthly injections of the miR-10b-5p mimic, whereas levels rapidly dropped after miR-10b-5p inhibitor injection. (FIG. 9H) Consistently, the levels of insulin and C-peptide followed similar patterns to those of miR-10b-5p in miR-10b-5p mimic- or inhibitor-injected mice. (FIGS. 39I and 39J) GI functions were severely impaired in miR-10b-5p inhibitor-injected mice fed either diet, while miR-10b-5p mimic injection protected against dysmotility in HFHSD-fed mice. The antagonistic targeting effects by the miR-10b-5p mimic and inhibitor in mice confirms that miR-10b-5p can regulate the development of diabetes and slow GI transit. Moreover, restoration of miR-10b-5p through miR-10b-5p mimic injection prevents the onset of these pathologies in HFHSD-fed mice

### Example 10—Conditional Removal of KIT⁺ Cells in Kit^{Cre-ERT2/+};Rosa26^{DTA/+} Male Mice Leads to Diabetes and Slow GI Transit

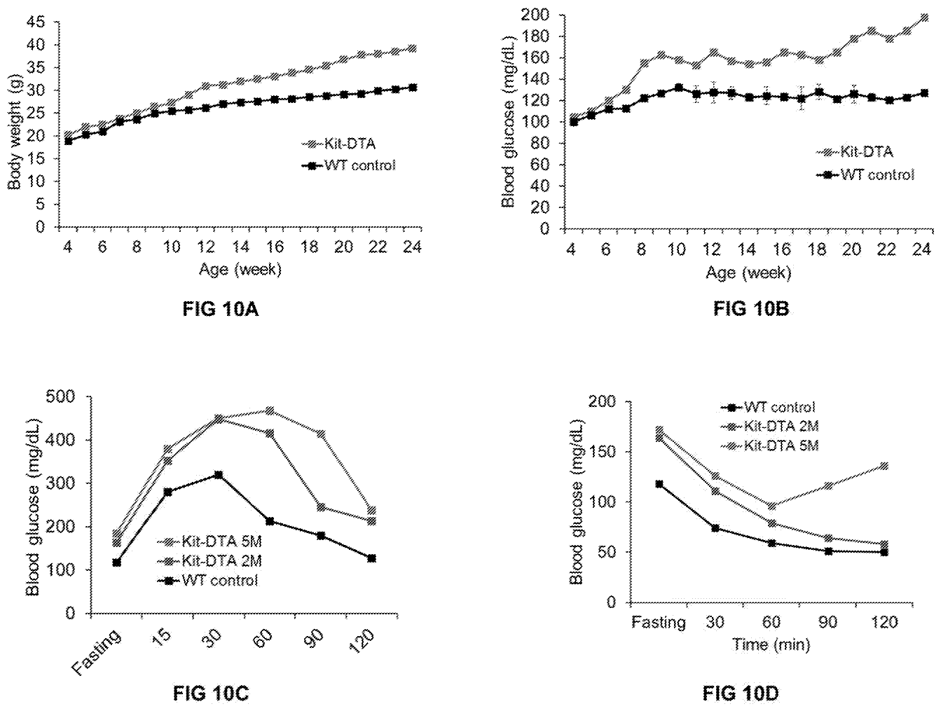

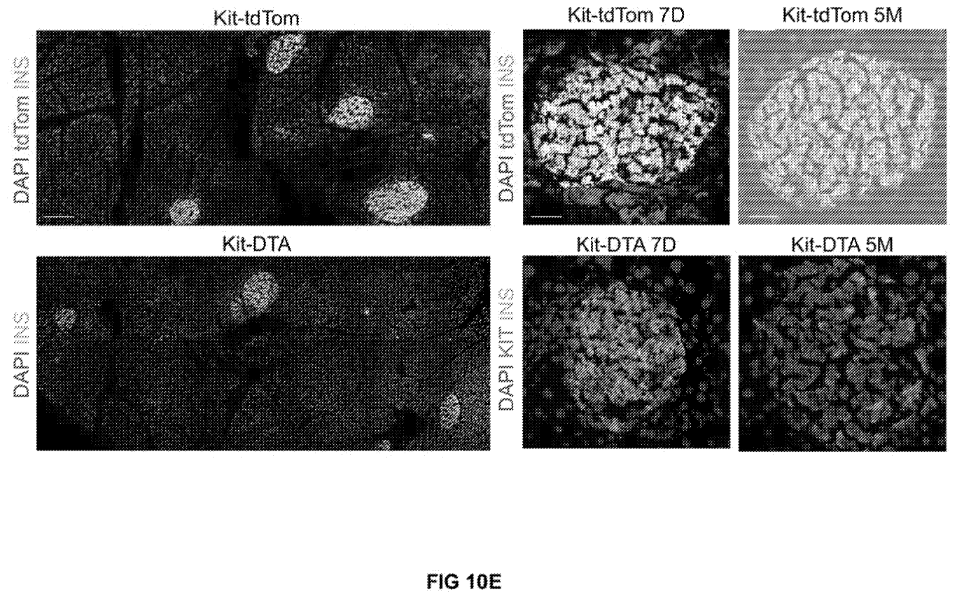

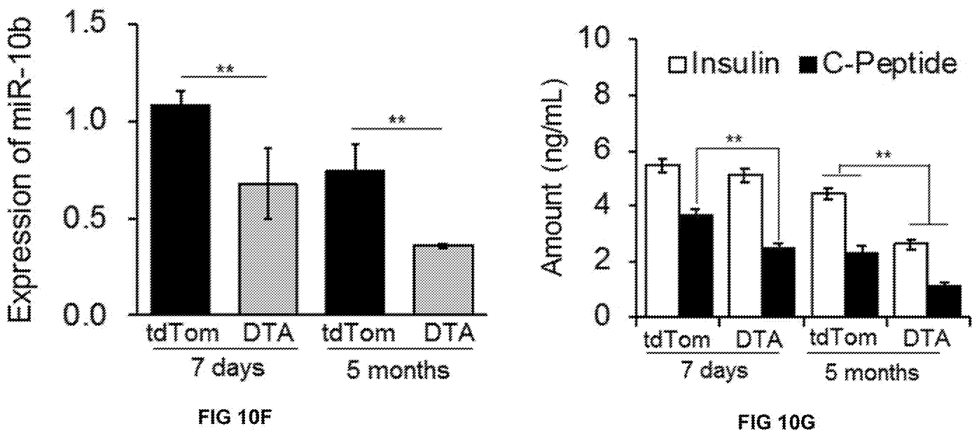

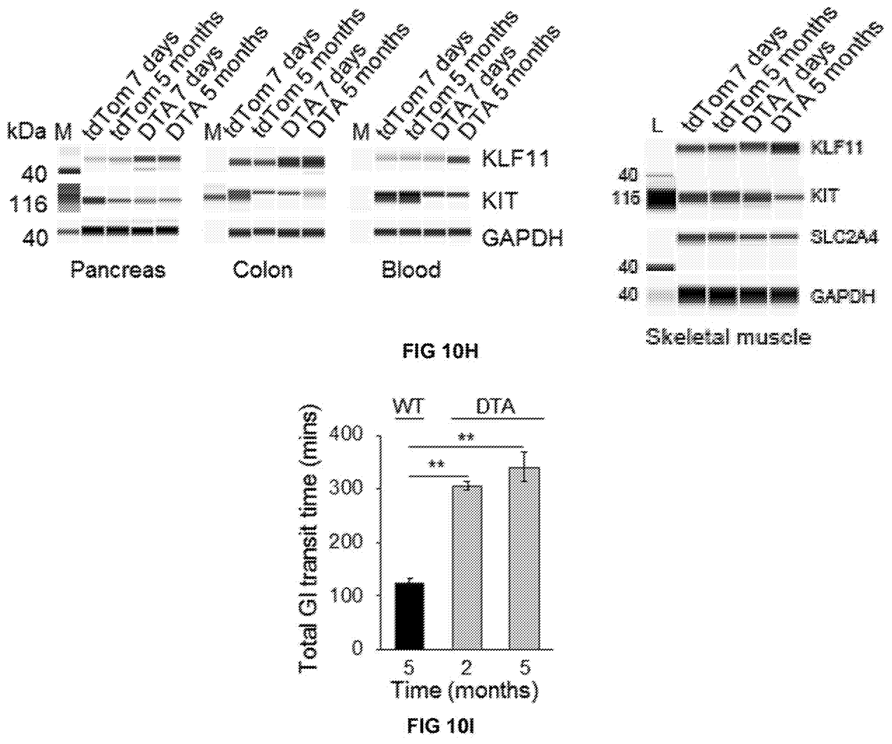

To examine loss of function of KIT⁺ cells in mice, the cells were conditionally removed with DTA in Kit^{CreERT2/+};Rosa26^{DTA/+} (Kit-DTA) and the cells were labeled with tdTomato in Kit^{CreERT2/+};Rosa26^{tdTom/+} (Kit-tdTom) by tamoxifen injection, respectively. Kit-DTA mice gained weight faster than WT controls after 4 weeks (FIG. 10A). Blood glucose noticeably increased after 6 weeks post-tamoxifen injection and became hyperglycemic levels at 24 weeks (FIG. 10B). GTT and ITT showed that glucose tolerance and insulin sensitivity in Kit-DTA mice have been gradually impaired at 2 and 5 months (FIGS. 10C-10D). A large number of KIT⁺ cells were identified in the pancreas of Kit-tdTom mice after 7 days (7D) post-tamoxifen injection (FIG. 10E). Many KIT⁺ cells (tdTom) appeared to be β cells (INS⁺) in islets at 7 days, but a few β cells appeared to be KIT⁺ at 5 months. This suggests that β cells were derived from KIT⁺ cells, but most of the β cells originated from the KIT⁺ precursors were lost and replaced with newly derived β cells at 5 months. It also suggests that a few KIT⁺ precursor-derived β cells are preserved up to 5 months. On the other hand, Kit-DTA mice lost KIT⁺ cells at 7 days and 5 months. The islet number and size were similar in the pancreas of the Kit-DTA mice at 5 months, but β cells were smaller in size and degenerated. MiR-10b was noticeably reduced in the blood of the Kit-DTA mice at 5 days and 5 months, compared to Kit-tdTom mice (FIG. 10F). Consistent with miR-10b reduction, both insulin and C-peptide were reduced slightly at day 5 and noticeably at 5 months (FIG. 10G). KLF11 was also increased in the blood, pancreas, and colon of the Kit-DTA mice at 7 days and 5 months while KIT was accordingly reduced (FIG. 10H). Furthermore, total GI transit time was severely delayed in the Kit-DTA mice at 2 and 5 months (FIG. 10I). The results are shown in FIGS. 10A-10I.

### Example 11—Expression of miR-10b-5p, KLF11, and KIT in Human Patients with T2D and Gastroparesis

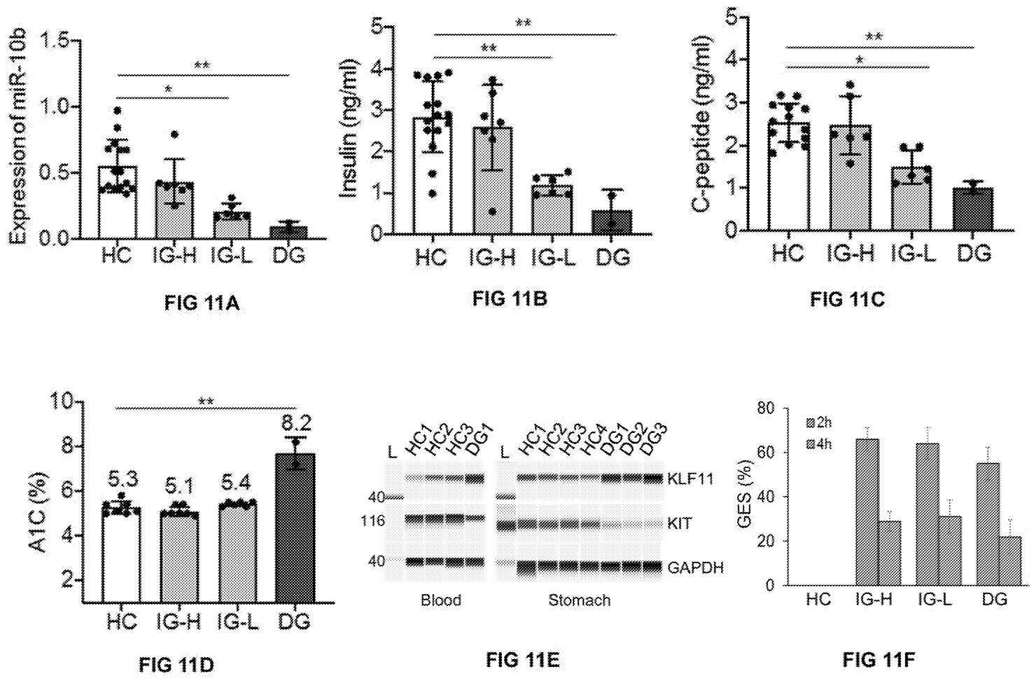

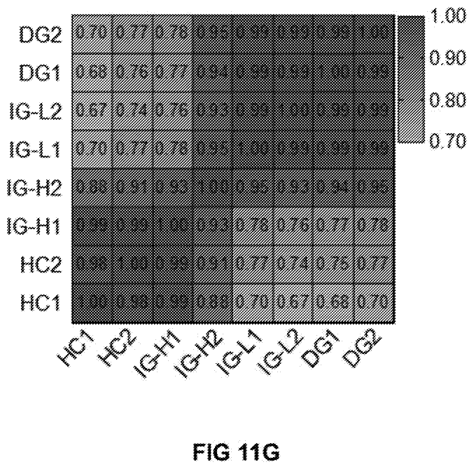

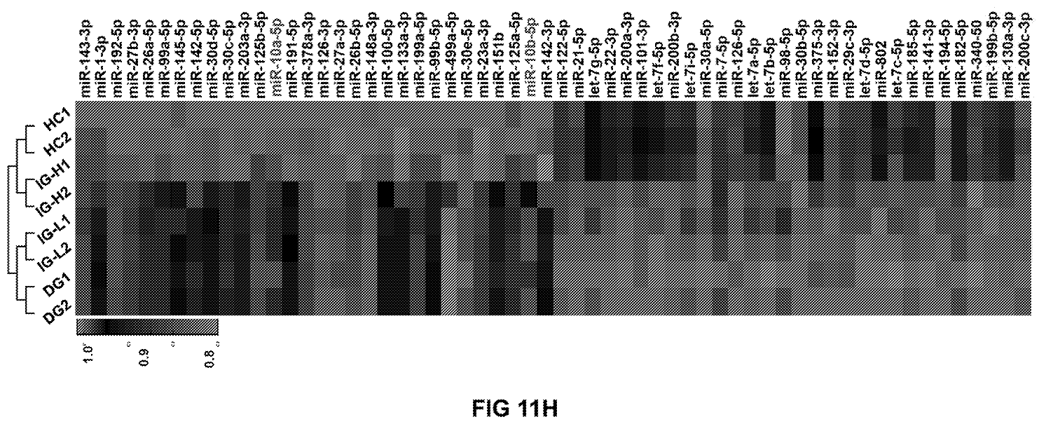

Next, samples from human patients with diabetic and idiopathic gastroparesis were examined to see if the abnormal expression patterns of miR-10b-5p, KLF11, and KIT found in the diabetic murine models are similar in human patient samples. Fifteen adult subjects (age 18-65) with a diagnosis of gastroparesis based on established guidelines and abnormal gastric emptying scintigraphy and 15 control subjects were used for the gene expression analysis. Based on miR-10b-5p expression levels, samples could be divided into four distinct groups: high expression, which corresponded to healthy control samples (HC); intermediated expression, which corresponded to idiopathic gastroparesis (IG) samples and were divided in higher and lower expression groups (IG-H and IG-L); and low expression, which corresponded to diabetic gastroparesis samples (DG) (FIG. 11A). Consistent with the data from mice, insulin and C-peptide levels showed correlative patterns to miR-10b-5p expression in human samples (FIGS. 11B and 11C). The IG-L group had significantly lower levels of miR-10b-5p, insulin and C-peptide than compared to those of the HC group, suggesting that the IG-L group is at a prediabetic stage. A1C levels of the IG-H and IG-H groups were similar to the HC group with a normal range (>5.7) (FIG. 11D), suggesting that A1C could not distinguish the prediabetic condition from the HC group. KLF11 protein levels in the blood serum and antrum were also higher in the DG group than in the HC group while KIT levels were lower in the DG group (FIG. 11E). Consistent with results found in mice, samples from human patients with T2D and/or gastroparesis showed decreased miR-10b-5p, increased KLF11, and decreased KIT. Deep sequencing of miRNAs from two individual blood samples of HC, IG-H, IG-L, and DG identified differentially expressed miRNAs. Expression patterns of the top 70 most dynamically regulated miRNAs show clustering between HC and IG-H and between IG-L and DG (FIGS. 11F and 11G). miR-10a-5p and miR-10b-5p levels are highest in HC, followed by IG-H, IG-L, & DG respectively (FIG. 11G). Taken together, expression levels of miR-10b-5p, KLF11, and KIT in HC and DG human patient samples are very similar to the mouse data obtained in this study. Additionally, expression levels of miR-10b-5p, insulin and C-peptide in IG-H and IG-L suggest these groups are in a pre-diabetic condition progressing into diabetes.

### Example 12—Diabetes Model of miR-10b Regulation

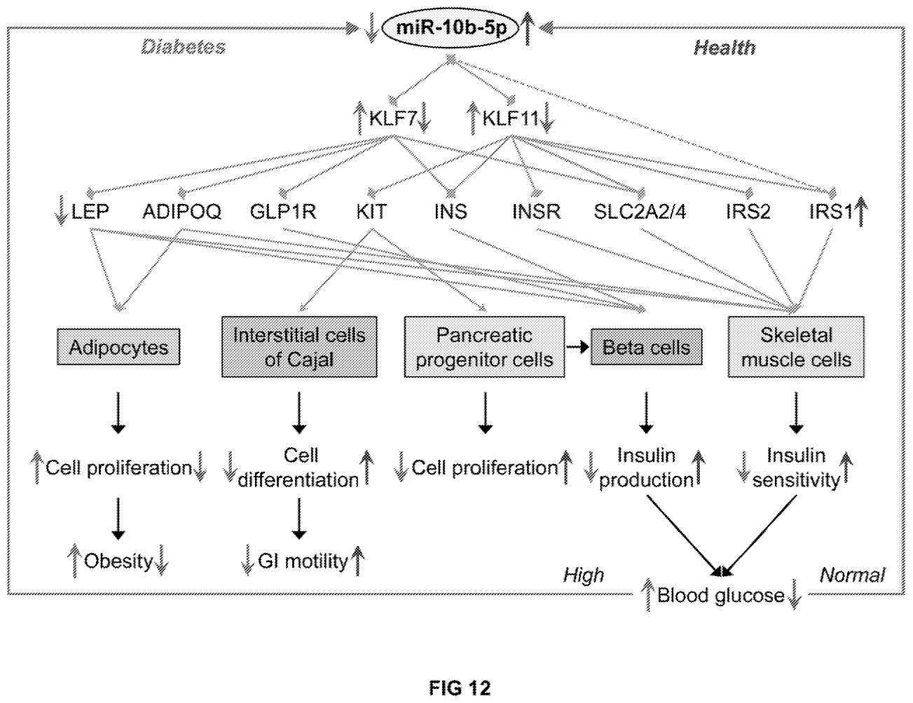

Without wishing to be bound by theory, based on data obtained from the present transgenic animal and human study, it seems that miR-10b regulates diabetes, GI motility and obesity (FIG. 12). MiR-10b-5p targets and suppresses the translation of two metabolic transcriptional factors, KLF7 and KLF11. These transcription factors negatively regulate the expression of metabolic genes (LEP, ADIPOQ, GLP1R, KIT, INS, INSR, SLC2A2, SLC2A4, IRS1, and IRS2) that dictate cell proliferation, differentiation, insulin production and sensitivity in adipocytes, ICC, pancreatic progenitor cells, beta cells, and skeletal muscle cells. MiR-10b is highly expressed under normal glucose level conditions, which suppresses KLF7 and KLF11, thereby allowing high expression of metabolic genes. However, in high glucose condition, miR-10b is silenced, thereby allowing the two transcription factors to escape, becoming over-expressed, which down-regulates the expression of metabolic genes, leading to the development of diabetes, GI dysmotility and obesity.

### Example 13: The miR-10a-5p Mimic Rescues the Obese and Diabetic Phenotype

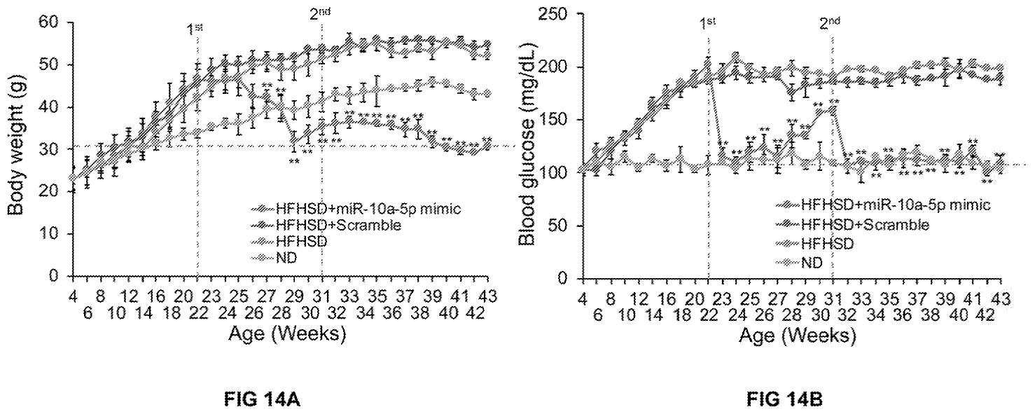

FIG. 13A. shows the sequence and structure of the mouse miR-10a precursor encoding miR-10a-5p and miR-10a-3p, a synthetic miR-10a-5p molecule (miR-10a-5p mimic) and a synthetic miR-10a-5p antisense molecule (miR-10a-5p inhibitor). FIG. 13B shows the sequence and structure of the mouse miR-10b precursor encoding miR-10b-5p and miR-10b-3p, a synthetic miR-10b-5p molecule (miR-10b-5p mimic) and a synthetic miR-10b-5p antisense molecule (miR-10b-5p inhibitor). Note a single nucleotide base difference between the miR-10a-5p mimic (U) and miR-10b-5p mimic (A) and also between the miR-10a-5p inhibitor (A) and miR-10b-5p inhibitor (U) are shown in bold letters. These constructs were then used in subsequent studies. The miR-10a-5p mimic rescues the obese and diabetic phenotype in mice fed a high-fat and high-sucrose diet (HFHSD) or a normal diet (ND). (FIGS. 14A and 14B) Body weight and fasting blood glucose comparison. Male C57 mice were fed a HFHSD for the entirety of the experiment (4-43 weeks) and injected twice (1^{st} injection at 22 weeks and 2^{nd} injection at 31 weeks) with either the miR-10a-5p mimic, negative control (a scramble RNA), or given no injection at 22 weeks and were monitored thereafter over a 21-week period, post-injection (PI). n=3-6 per group. *p<0.05 and **p<0.01 (miR-10a-5p mimic versus no injection in a HFHSD).

### Example 14: The miR-10a-5p Mimic Injection Results in Substantial Weight Loss and Reduces BMI and Waist Circumference

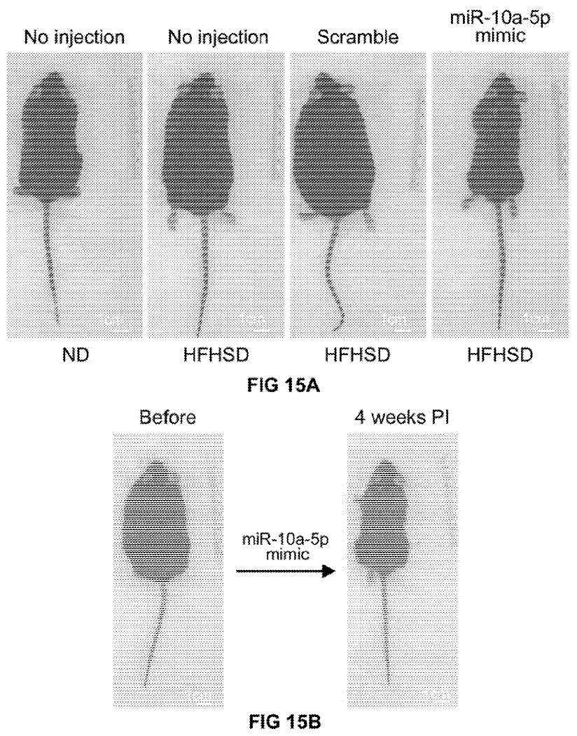

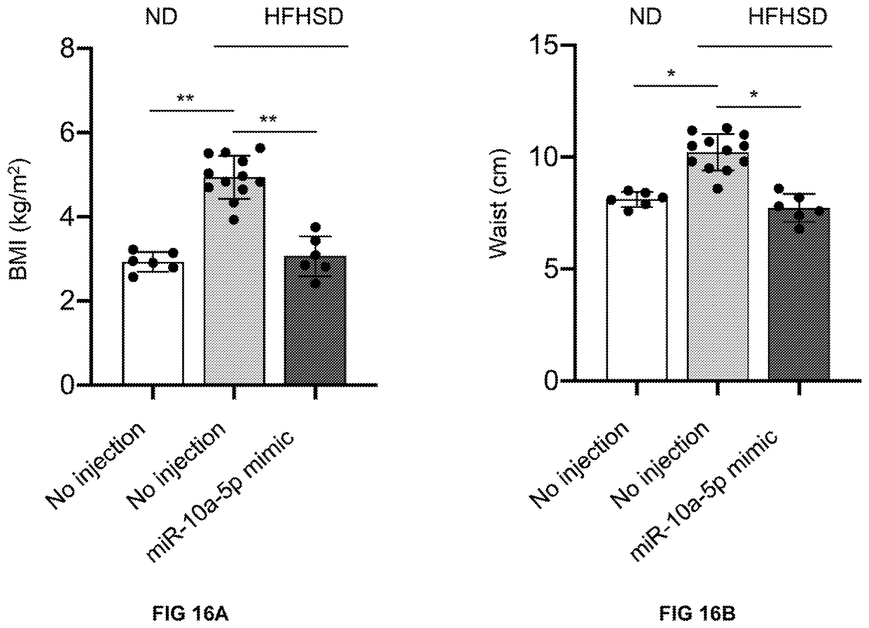

Male C57 mice were fed a HFHSD for the entirety of the study and treated with two injections of miR-10a-5p mimic, negative control, or no injection in a manner similar to the previous study. FIG. 15A shows the gross anatomical changes of HFHSD-induced obese and diabetic mice injected with miR-10a-5p mimic, negative control (a scramble RNA), or no injection, indicated in FIG. 14 (mice at 12W PI after 1^{st} injection at 43W). (FIG. 15B) A gross anatomical change of the same HFHSD-induced obese and diabetic mouse injected with miR-10a-5p mimic at 22 weeks before injection and at 4 weeks PI. Changes of average BMI and waist circumference (FIGS. 15A and 15B) were also assessed. n=3 per each group. *p<0.05 and **p<0.01. Changes of average BMI and waist circumference (FIGS. 16A and 16B) in HFHSD-induced obese and diabetic mice injected with miR-10a-5p mimic, negative control (a scramble RNA), or no injection, compared to healthy mice fed a ND and give no injection in FIG. 14 (measured twice at 8 weeks PI after 1^{st} injection at 23 weeks and at 1 week PI after 2^{nd} injection at 31 weeks). n=6-12 per each group. *p<0.05 and **p<0.01. In all, this study demonstrated a weight loss of 40% and improved glucose and insulin tolerance in diabetic mice receiving the 10a mimic.

### Example 15: miR-10a-5p Mimic Injection Restores Insulin Levels in HFHSD-Induced Diabetic Mice

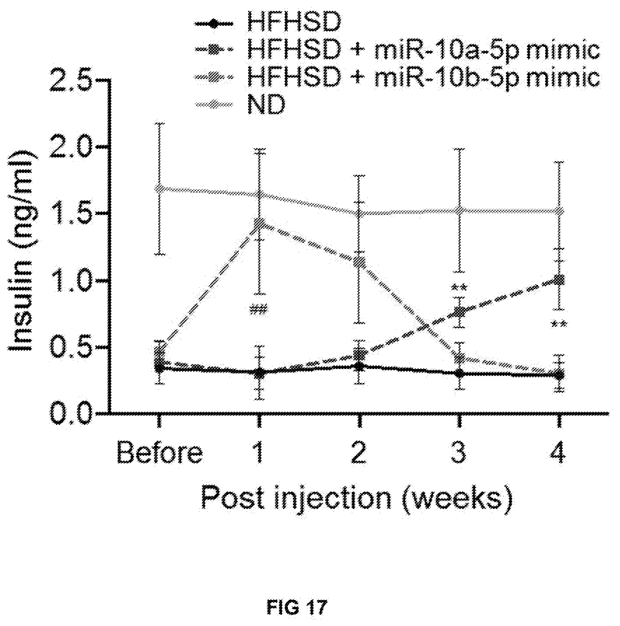

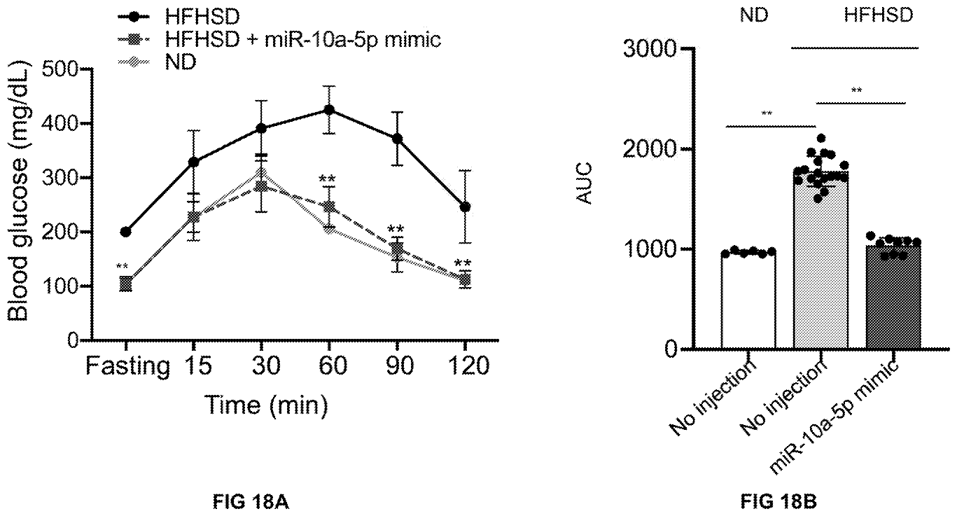

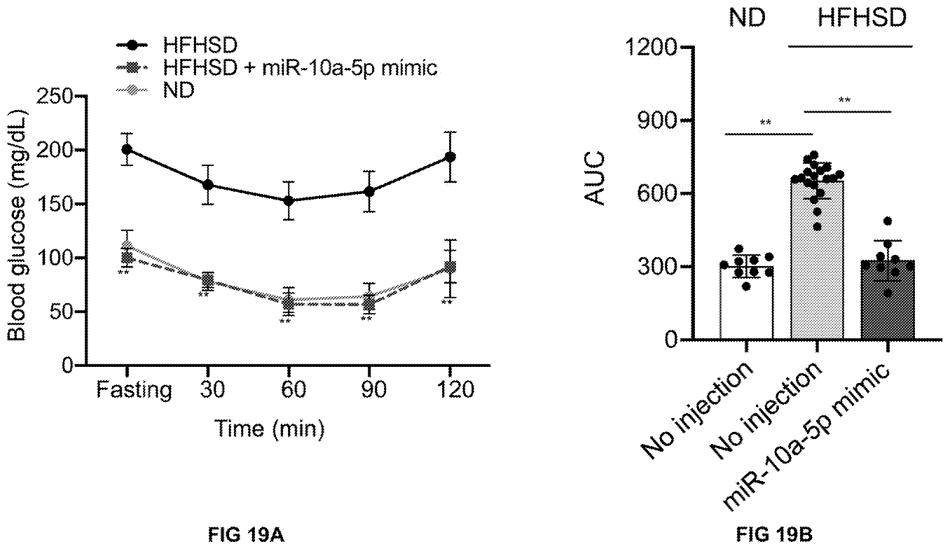

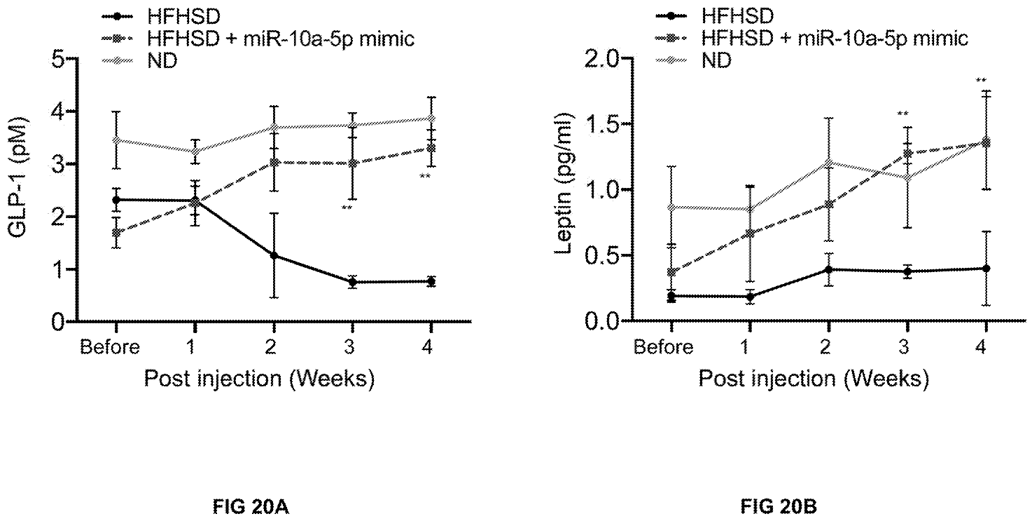

While the miR-10b-5p mimic injection increases insulin levels, the effects of miR-10a-5p on insulin levels was then examined. Changes in insulin levels were assessed after 6 h of fasting in male mice fed a HFHSD or a normal diet (ND) and injected with either a miR-10a-5p mimic, miR-10b-5p mimic, negative control (a scramble RNA), or no injection (measured at 2W PI after 2^{nd} injection at 31W). (FIG. 17) n=6-12 per condition for each experiment. Follow-up studies then assessed changes in insulin over the course of four weeks after injection with either miR-10b-5p or miR-10a-5p. An inverse regulation of insulin by miR-10a-5p mimic and miR-10b-5p mimic was observed (FIG. 17). miR-10a-5p mimic injection slightly decreases and slowly restores insulin levels in HFHSD-induced diabetic mice while the miR-10b-5p mimic injection transiently increases and gradually decreases insulin levels. Changes in insulin levels after 6 h of fasting in male mice fed a HFHSD or a ND and injected with either a miR-10a-5p mimic, miR-10b-5p mimic, or no injection over 4 weeks. n=6-12 per condition for each experiment. **p<0.01 (miR-10a-5p mimic versus no injection in a HFHSD); ##p<0.01 (miR-10b-5p mimic versus no injection in a HFHSD). The ability of the miR-10a-5p mimic to improve the glucose and insulin tolerance of HFHSD-induced diabetic mice was then assessed. (FIG. 18A) Intraperitoneal (IP) glucose tolerance tests (GTT) at 4 weeks after injection of miR-10a-5b mimic or no injection. (FIG. 18B) GTT plot of the area under the curve (AUC). n=6-12 per each group. **p<0.01. A similar study assessed insulin tolerance. (FIG. 19A) IP insulin tolerance tests (ITT) at 4 weeks after 1^{st} injection of miR-10a-5p mimic. (FIG. 19B) ITT plot of the area under the curve (AUC). n=6-12 per each group. **p<0.01. The effects of miR-10a-5p treatment on insulin-related metabolism-regulating hormones GLP-1 and leptin. miR-10a-5p mimic injection was found to increase (FIG. 20A) GLP-1 and (FIG. 20B) Leptin in HFHSD-induced diabetic mice. miR-10a-5p mimic injection increases and restores (FIG. 20A) GLP-1 and (FIG. 20B) Leptin in HFHSD-induced diabetic mice to the hormone levels close to healthy mice fed a ND over 4 weeks. Increased signaling of both of these hormones is known to reduce body weight. n=4 per each group for each experiment. **p<0.01 (miR-10a-5p mimic versus no injection in a HFHSD). Together, these results demonstrated a significant restoration of insulin levels in miR-10a-5p mimic treated mice to those similar to non-diabetic mice, and without wishing to be bound by theory suggested that treatment of diabetic obesity with miR-10a-5p mimic can reverse multiple symptoms of the condition.

### Example 16: miR-10a-5p Mimic Injection Improves GI Function

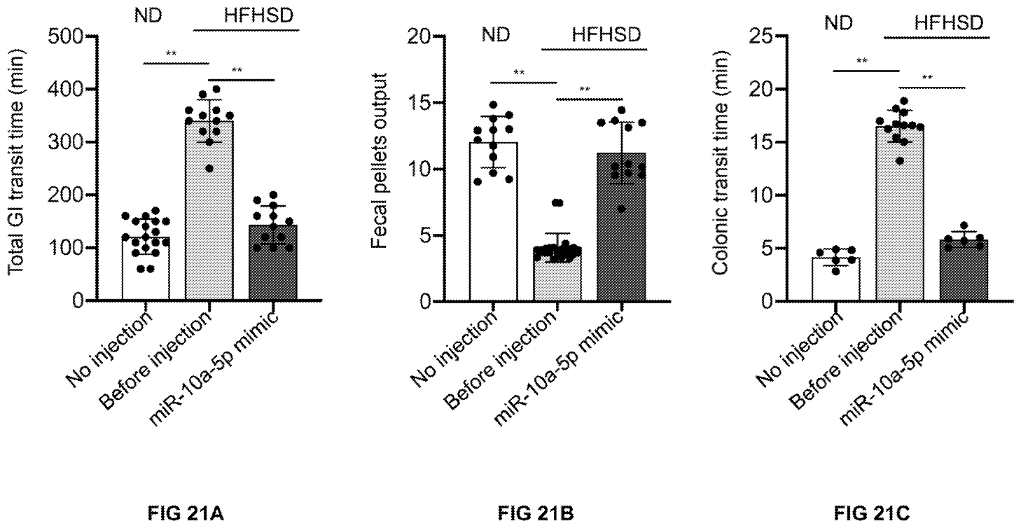

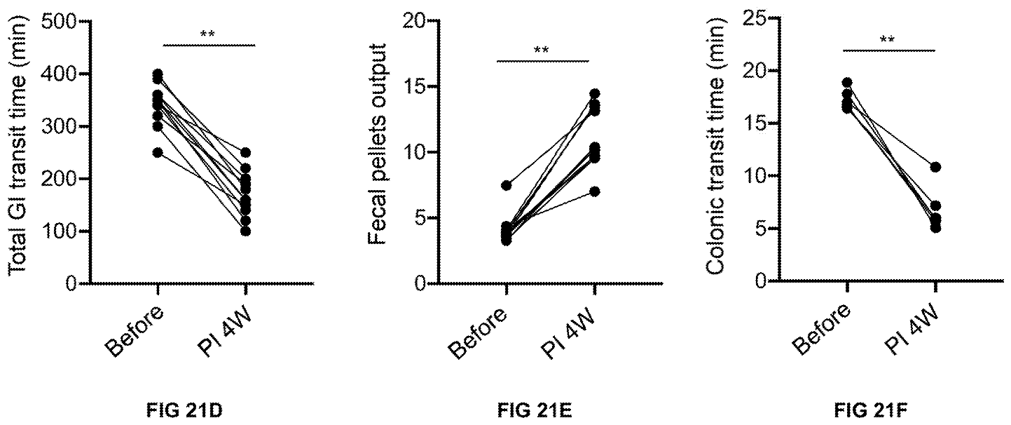

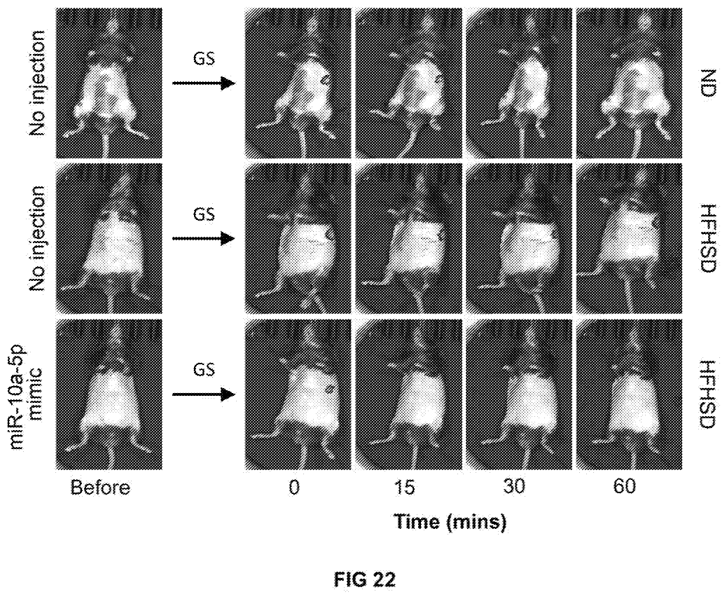

Male C57BL/6 mice fed a HFHSD were assessed for GI function after treatment with a miR-10a-5p mimic, a scramble negative control, or no injection. (FIG. 21A-21C) Total GI transit time, fecal pellet output, and colon transit time was assessed (FIGS. 21A and 21B, measured at 8W PI after 1^{st} injection at 23W; FIG. 21C, at 2W PI after 2^{nd} injection at 31W). A follow-up study assessed total GI transit time (FIG. 21A), fecal pellet output (FIG. 21B), and colon transit time (FIG. 21C) in male diabetic mice fed a HFHSD or male healthy mice fed ND and injected with either a miR-10a-5p mimic, or no injection at 4 weeks PI. (FIGS. 21D-21F) Changes of total GI transit time, fecal pellet output, and colon transit time in individual mice that were in FIG. 21A-21C. n=6-18 per each group. **p<0.01. As an additional measure of gastric function, the rate of gastric emptying was measured in male diabetic mice fed a HFHSD or male healthy mice fed a ND and injected with either a miR-10a-5p mimic or no injection. Fluorescent GastroSense 750 (GS) mixed food was applied to the stomach by oral gavage and then imaged by an IVIS Lumina III system at 0 and 60 minutes post gavage (FIG. 22). These data demonstrated that treatment with 10a-mimic could reduce GI transit time, increase fecal pellet output, increase gastric emptying, and significantly reduced colonic transit time. In total these observations suggested that not only does treatment with miR-10a-5p mimic result in the reversal of diabetic obesity, but also improves GI function.

### Example 17: Both miR-10a-5p Mimic and miR-10b-5p Mimic Protect Against an Obese and Diabetic Phenotype

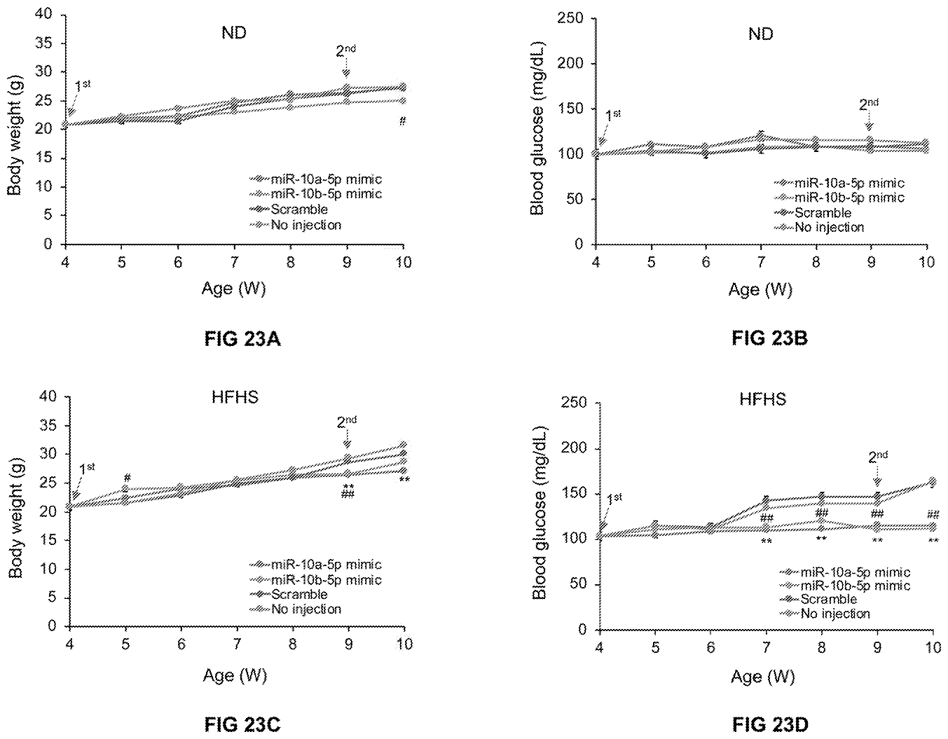

To see if treatment with miR-10a-5p and miR-10b-5p mimics could prevent the development of obesity and diabetes in addition to treating already-existing conditions, male C57BL/6 mice were fed a HFHSD diet and then injected twice (1^{st} and 2^{nd} injection) with a miR-10a-5p mimic, miR-10b-5p mimic, negative control (a scramble RNA), or no injection, and then fed a HFHSD or ND. (FIGS. 23A and 23B) Body weight and fasting blood glucose in mice fed a ND. (FIGS. 23C and 23D) Body weight and fasting blood glucose in mice fed a HFHSD. n=3 per each group. *p<0.05 and **p<0.01 (miR-10a-5p mimic versus no injection); #p<0.05 and ##p<0.01 (miR-10b-5p mimic versus no injection). These data showed the pre-treatment with both mimics could significantly reduce the onset of obesity and maintain lower blood glucose levels as compared to controls. In mice fed a normal diet, only miR-10b-5p treatment resulted in a significant delay in body weight gain, though there were no accompanying differences in blood glucose levels. Further study of ND-fed mice revealed that miR-10a-5p mimic and/or miR-10b-5p mimic injection into mice fed a ND do not change fasting glucose levels at 2W PI after 2^{nd} injection at 9W. n=3 per each group (FIG. 23B).

### Example 18: Treatment with miR-10a-5p and miR-10b-5p Inhibitors Trigger and Exacerbate the Obese and Diabetic Phenotype in Mice

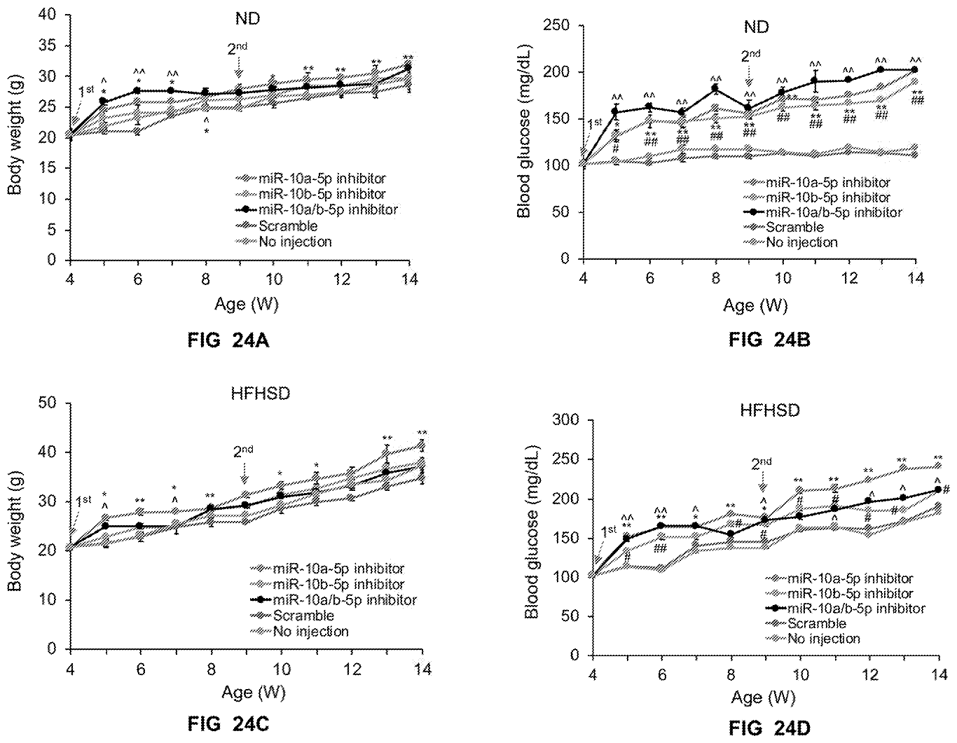

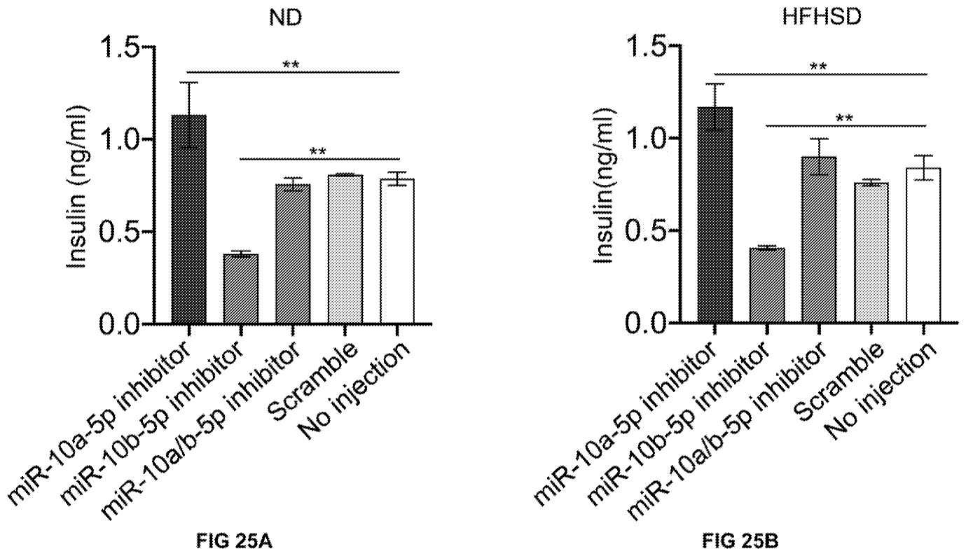

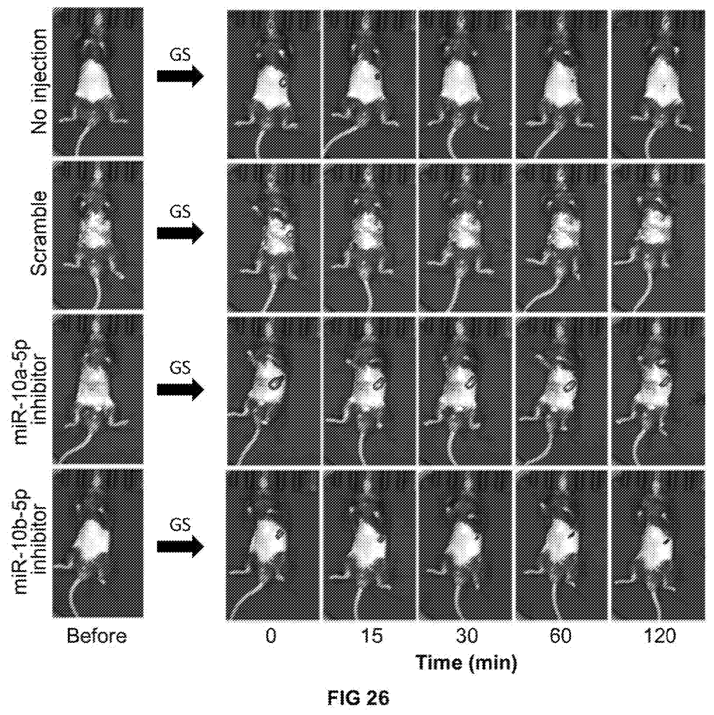

Having observed that miR-10a-5p and miR-10b-5p mimic treatment could reduce and prevent the obese and diabetic phenotype in mice, studies were then conducted to see if treatment with inhibitors based on the sequences of miR-10a-5p and miR-10b-5p could have the opposite effect (displayed in FIG. 23). Male C57BL/6 mice were fed ND or HSHSD and then injected twice at 4W and 9W (1^{st} and 2^{nd} injection) with either the miR-10a-5p inhibitor, miR-10b-5p inhibitor, both miR-10a-5p inhibitor and miR-10b-5p inhibitor (miR-10a/b-5p inhibitor), negative control (a scramble RNA), or no injection, and then fed a HFHSD or a normal diet (ND). (FIGS. 24A and 24B) Body weight and fasting blood glucose in mice fed a ND. (FIGS. 24C and 24D) Body weight and fasting blood glucose in mice fed a HFHSD. n=3 per group. *p<0.05 and **p<0.01 (miR-10a-5p inhibitor versus no injection), #p<0.05 and ##p<0.01, (miR-10b-5p inhibitor versus no injection), \hat{ }p<0.05 and \hat{ }\hat{ }p<0.01 (miR-10a/b-5p inhibitor versus no injection). Inhibitor effects on fasting insulin levels was then assessed. miR-10a-5p inhibitor increases fasting insulin levels while miR-10b-5p inhibitor decreases fasting insulin levels in male healthy mice fed a ND (FIG. 25A) and male diabetic mice fed a HFHSD (FIG. 25B) at 2 weeks PI after 2^{nd} injection at 9 weeks. **p<0.01. Additionally, gastric emptying was observed in inhibitor-treated mice. (FIG. 26) Images in mice fed a ND at 4 weeks PI after 2^{nd} injection with either the miR-10a-5p inhibitor, miR-10b-5p inhibitor, negative control (a scramble RNA), or no injection at 10 weeks. GS denotes GastroSense 750. Together these data demonstrated an accelerated onset of diabetes and weight gain in mice fed both types of diet. Treatment with miR-10a-5p and miR-10b-5p inhibitors trigger and exacerbate the obese and diabetic phenotype in mice.

### Example 19: Genetic Targets of miR-10a-5p and miR-10b-5p

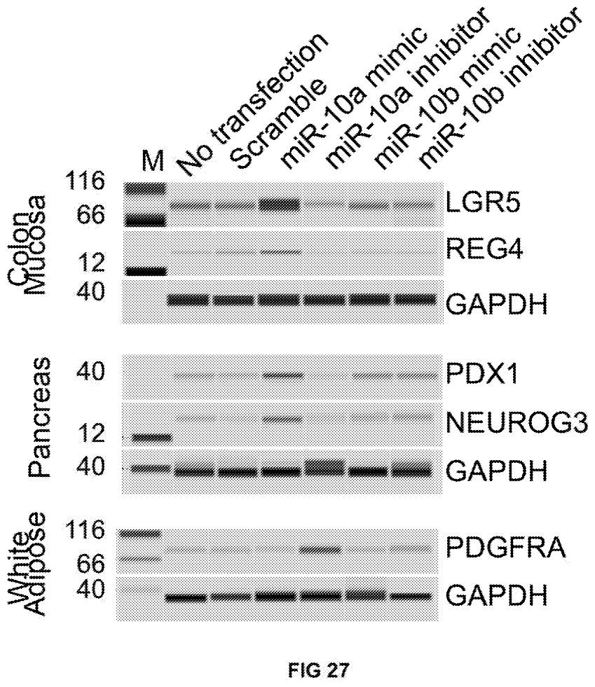

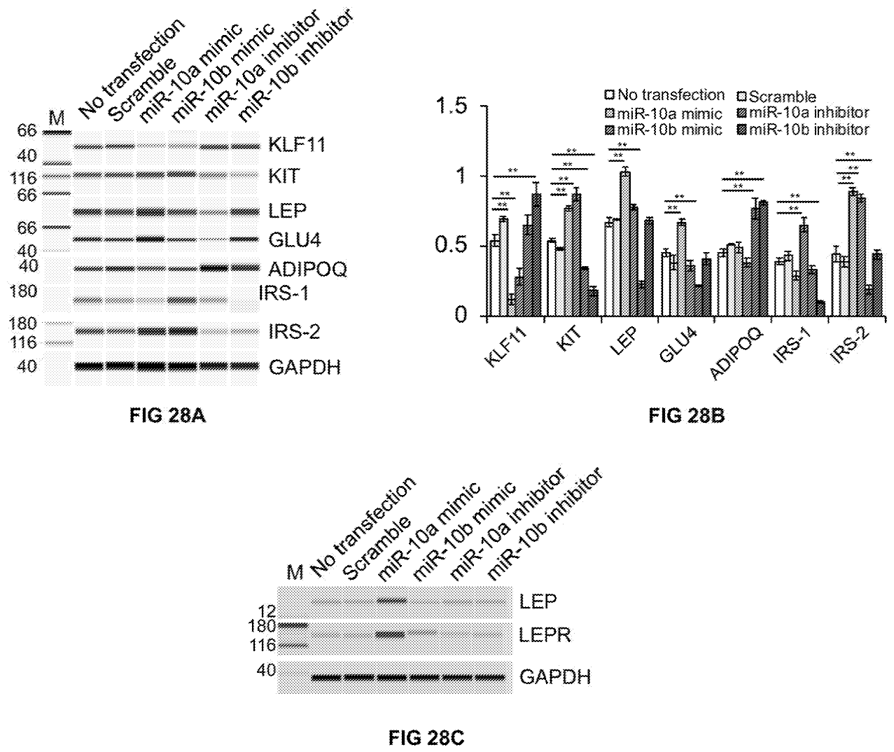

A series of studies was then undertaken to study the effects of miR-10a-5p and miR-10b-5p mimics on target gene expression. (FIG. 27) Observations indicated that miR-10a-5p and miR-10b-5p differentially target key proteins (LGR5, REG4, PDX1, NEROG3, and PDGFRA) in regulating stem cells or progenitor cells in colon mucosa, pancreas, and white adipocytes in mice fed a ND at 4 weeks PI after 1^{st} injection. GAPDH was used as an endogenous control in this western blot. Subsequent studies demonstrated that miR-10a-5p and miR-10b-5p differentially target the same obesity and diabetes-linked proteins. Comparison of targeting effects on obesity and diabetes-linked proteins by miR-10a-5p mimic, miR-10b-5p mimic, miR-10a-5p inhibitor and miR-10b-5p inhibitor. Mouse pancreas β-cells (NIT-1 cells) or mouse adipocytes (L-M cells) were transfected with miR-10a-5p mimic, miR-10b-5p mimic, miR-10a-5p inhibitor, miR-10b-5p inhibitor, negative control (a scramble RNA), or given no transfection. (FIG. 28A) Western blots of KLF11, KIT, LEP, GLU4, ADIPOQ, IRS-1, IRS-2, and GAPDH control in MT-1 cells. A protein marker (M) with corresponding molecular weights (kDa) is shown. (FIG. 28B) Quantification of proteins in A normalized by GAPDH. (FIG. 28C) Western blots of LEP, LEPR, and GAPDH control in L-M cells. n=3 per each group. *p<0.05 and **p<0.01.

### Example 20: miR-10a-5p Rescues Diabetes-Related Non-Alcoholic Fatty Liver Disease

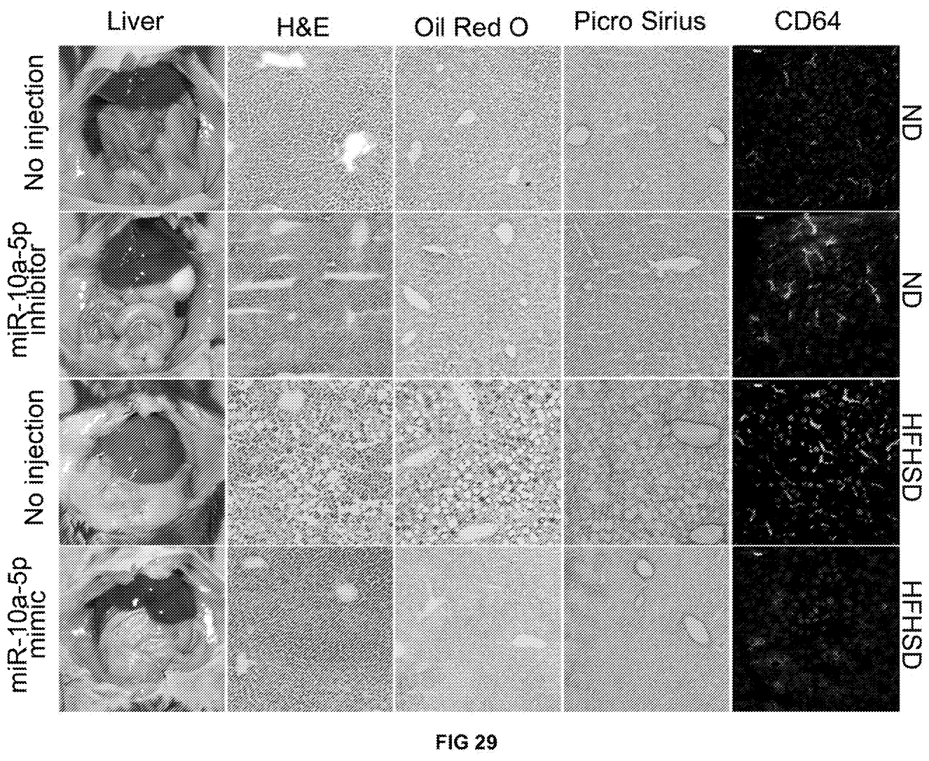

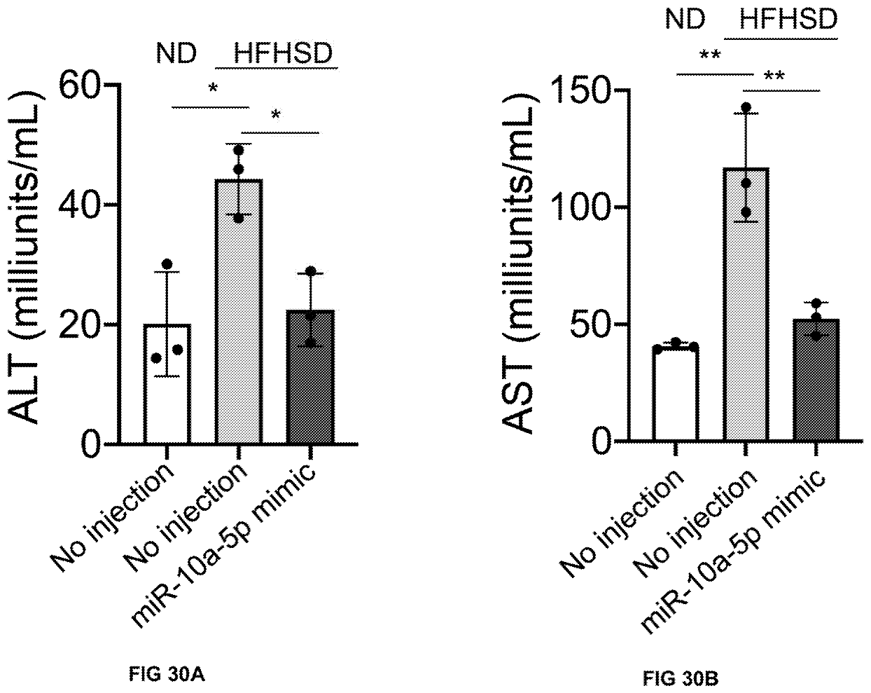

Non-alcoholic fatty liver disease (NAFLD) is highly prevalent in patients with type 2 diabetes mellitus, and correlates with associated obesity and insulin resistance. Using the HFHS mouse model from the previous examples, a series of studies was undertaken to determine whether treatment with miR-10a-5p mimic could prevent or reverse the fatty, inflammatory, and fibrosis liver phenotype in diabetic mice fed a HFHSD. (FIG. 29) Male diabetic C57BL/6 mice were fed a HFHSD or male healthy mice fed a ND for 18 weeks (4-22 weeks) were injected once at 22 weeks with miR-10a-5p mimic, miR-10a-5p inhibitor, or given no injection while continuing feeding the same diet. Gross and staining images of liver in the mice are shown. Liver tissue was dissected at 2 weeks PI from HFHSD mice and/or ND mice and stained with H&E staining, Oil Red 0 Staining (lipid), Picro Sirius Red Staining (fiber), and CD64 (macrophage). Similar studies were conducted, which assessed the levels of liver damage-related markers in treated vs untreated mice. (FIGS. 30A and 30B) Blood test for aspartate aminotransferase (AST) and alanine aminotransferase (ALT) in male diabetic mice fed a HFHSD or male healthy mice fed a ND for 18 weeks injected once at 22 weeks with miR-10a-5p mimic or given no injection while continuing feeding the same diet. AST and ALT was measured at 2 weeks PI from HFHSD mice and/or ND mice. n=3 per group. *p<0.05 and **p<0.01.

### Example 21: miR-10a-5p Treatment Rescues High Cholesterol and Reduces Inflammation in Diabetic Mice

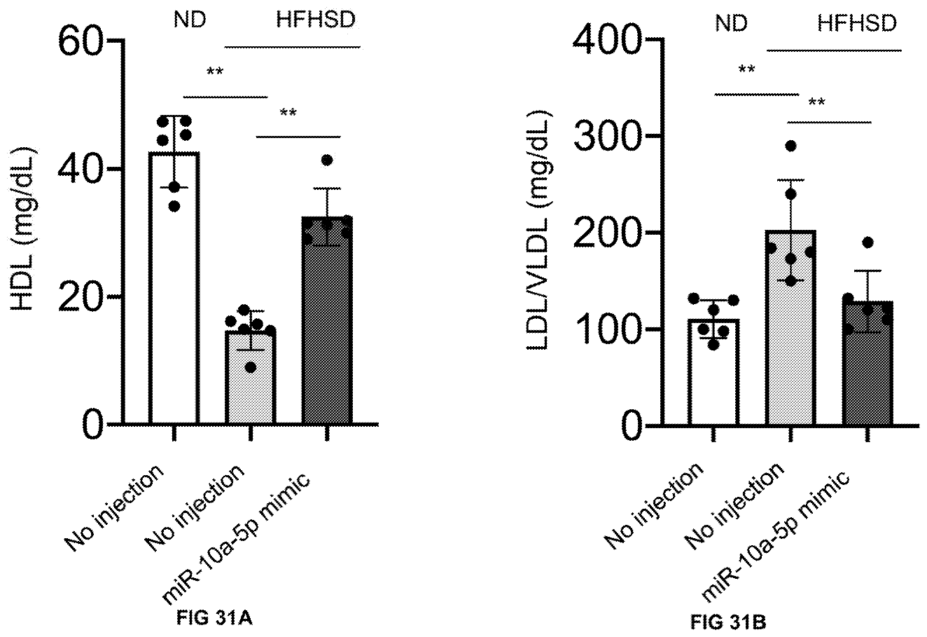

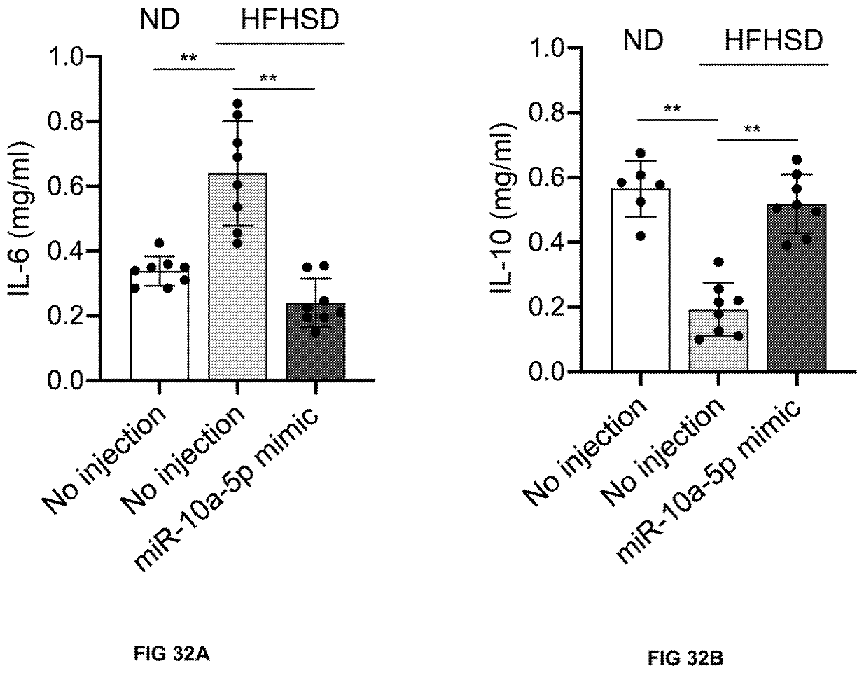

In addition to dysregulation of insulin and glucose, abnormal GI function, and development of fatty liver disease, diabetes is known to associate with high levels of “bad” LDL cholesterol. Treatment with miR-10a-5p mimic was found to rescues high cholesterol in diabetic mice fed a HFHSD. (FIGS. 31A and 31B) Blood test for high-density lipoprotein (HDL) and low-density lipoprotein (LDL)/very low-density lipoprotein (VLDL) cholesterol in male diabetic mice fed a HFHSD or male healthy mice fed a ND for 18 weeks and injected once at 22 weeks with miR-10a-5p mimic or given no injection while continuing feeding the same diet. HDL and LDL/VLDL were measured at 2 weeks PI from HFHSD mice and/or ND mice. n=6 per group. **p<0.01. Studies have also found that levels of inflammation-associated cytokines are often higher in patients having type 2 diabetes. In particular, these patients tend to have high levels of IL-6, a pro-inflammatory cytokine, and lower levels of IL-10, an anti-inflammatory cytokine. Treatment of HFHSD mice with miR-10a-5p mimic was found to reduce inflammation in diabetic mice fed a HFHSD. (FIGS. 32A and 32B) Blood test for pro-inflammatory cytokine, IL-6 and anti-inflammatory cytokine, IL-10 in male diabetic mice fed a HFHSD or male healthy mice fed a ND for 18 weeks and injected once at 22 weeks with miR-10a-5p mimic or given no injection while continuing feeding the same diet. IL-6 and IL-10 were measured at 2 weeks PI from HFHSD mice and/or ND mice. n=6-8 per group. **p<0.01.

### Example 22: Improvement of Heart Function in Diabetic Mice Treated with miR-10b-5p

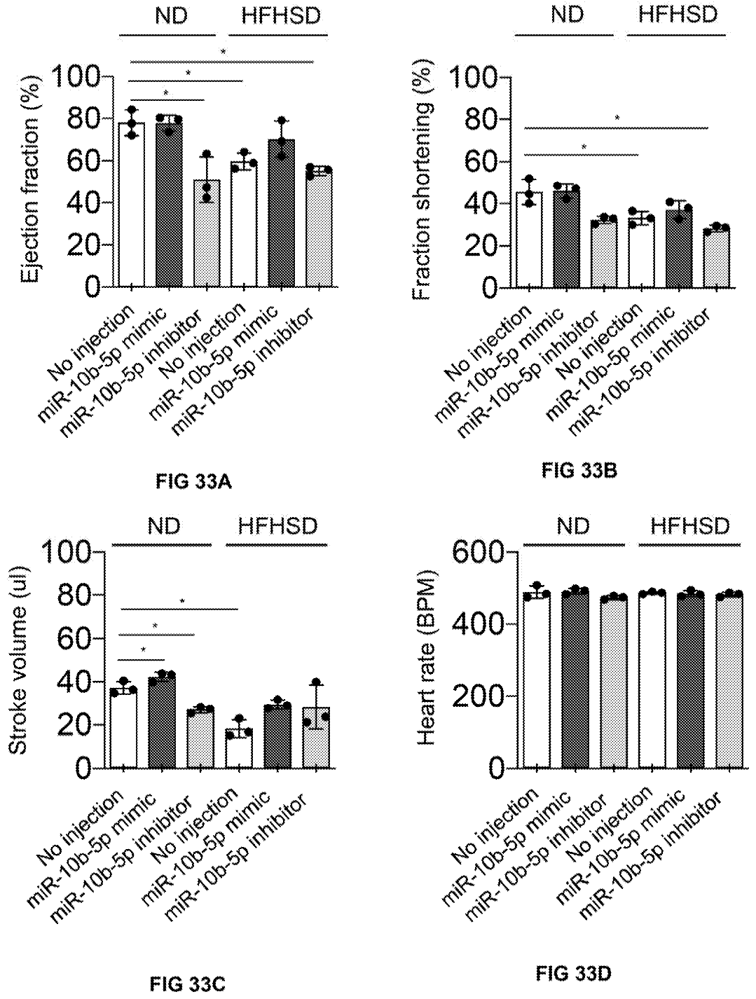

Abnormal heart function and diabetes are closely related. Patients with diabetes have an increased risk of developing heart disease and heart failure. Likewise, patients with heart failure often develop diabetes as a complication. In order to determine whether treatment with miR-10b-5p mimic improves heart functions, a series of studies were conducted in HFHSD-induced diabetic mice. Cardiac functions in male diabetic mice fed a HFHSD and male healthy mice fed a ND injected with miR-10b-5p mimic, miR-10b-5p inhibitor, or given no injection were evaluated using echocardiography. (FIG. 33A) Ejection fraction (EF). (FIG. 33B) Factional shortening (FS). (FIG. 33C) Stroke volume (SV). (FIG. 33D) Heart rate (HR). Diabetic mice fed HFHS (no injection) showed reduced EF, FS, and SV, compared to those fed a ND (no injection). miR-10b-5p mimic injection in mice fed a ND did not change EF and FS, but improved SV. miR-10b-5p inhibitor injection in mice fed a ND reduced EF and FS, and SV. miR-10b-5p mimic or inhibitor injection in mice fed a HFHSD did not significantly change EF, FS, and SV. A heart rate of 400 to 500 beats/min was maintained in all the mice during echocardiography. n=3 per group. *p<0.05 and **p<0.01.

### Example 23: Comparison of miR-10a-5p and miR-10b-5p Treatment with Established Diabetes Drugs

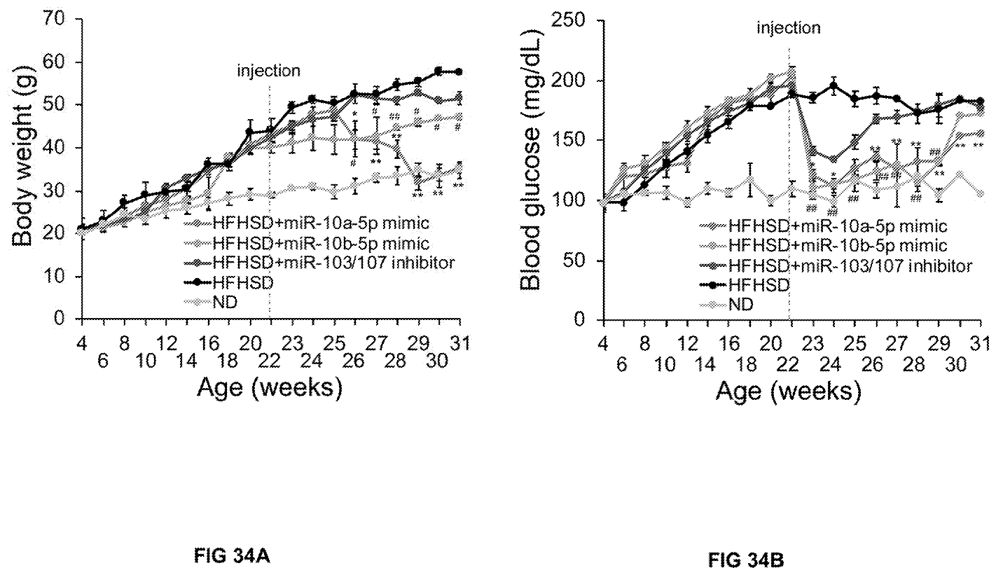

The relatively common occurrence of diabetes and diabetes-related diseases has led to the development of a number of drugs designed to counteract aspects of this disease. The relative efficacy of miR-10a-5p and miR-10b-5p mimics as compared to these established chemotherapies would give an indication of the clinical utility. A series of studies was then undertaken which compared the drug effects of miR-10a-5p, miR-10b-5p, and an investigational miR-103/107 inhibitor (RG-125) on body weight and blood glucose. The miR-10a-5p mimic rescues the obese and diabetic phenotype in mice fed a HFHSD. (FIGS. 34A and 34B) Body weight and fasting blood glucose comparison. Male C57BL/6 mice were fed a HFHSD or a ND for the entirety of the experiment and injected with either the miR-10a-5p mimic, miR-10b-5p mimic, miR-103/107 inhibitor, or given no injection at 22 weeks and monitored thereafter over a 9-week period, PI. AstraZeneca collaborating with Regulus Therapeutics has completed clinical trial phase 1 and 2 using miR-103/107 inhibitor (RG-125) in patients with type 2 diabetes and non-alcoholic fatty liver disease. miR-10a-5p and miR-10b-5p have greatly improved and lasting effects in lowering blood glucose and body weight than miR-103/107 inhibitor. n=3-6 per group. *p<0.05 and **p<0.01 (miR-10a-5p mimic versus miR-103/7 inhibitor), #p<0.05 and ##p<0.01, (miR-10b-5p mimic versus miR-103/7 inhibitor).

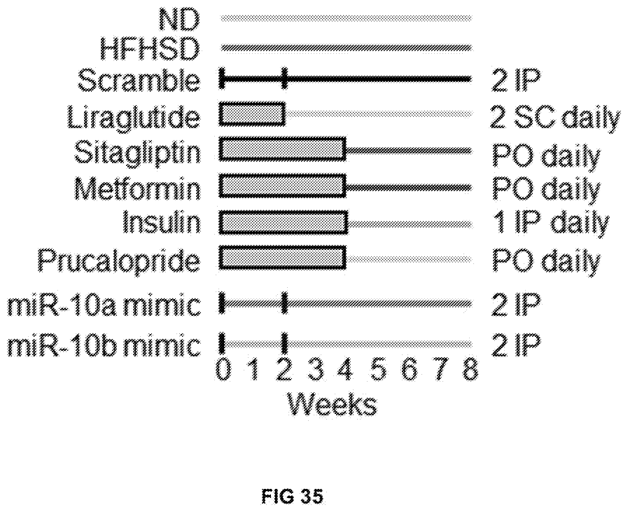

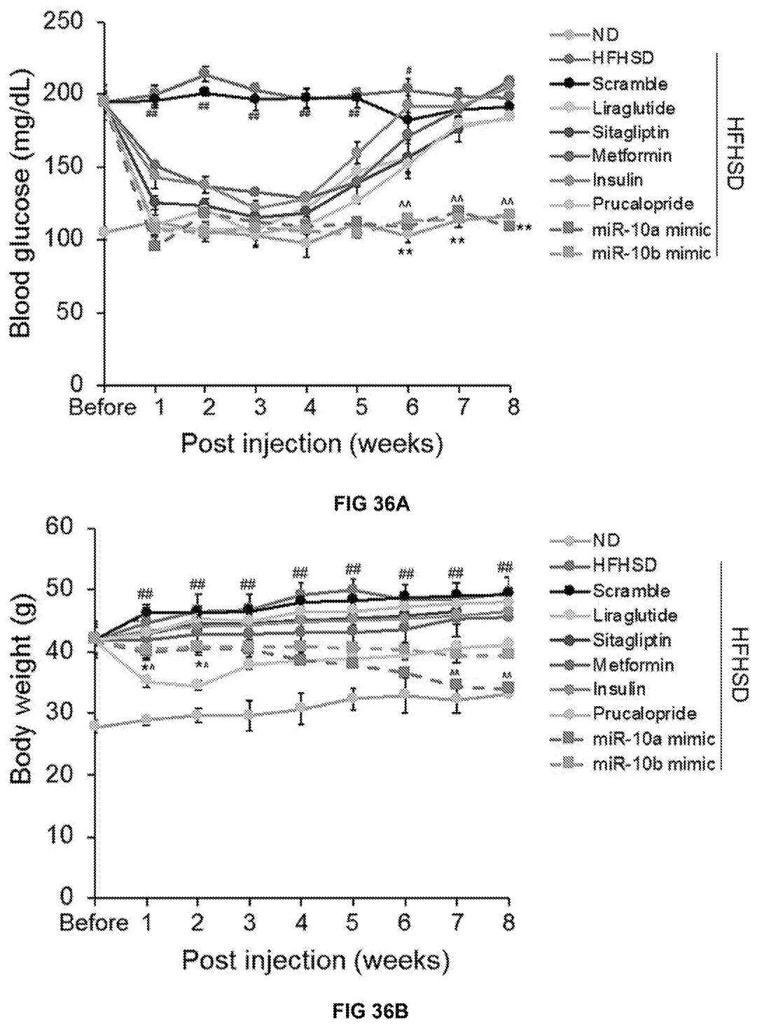

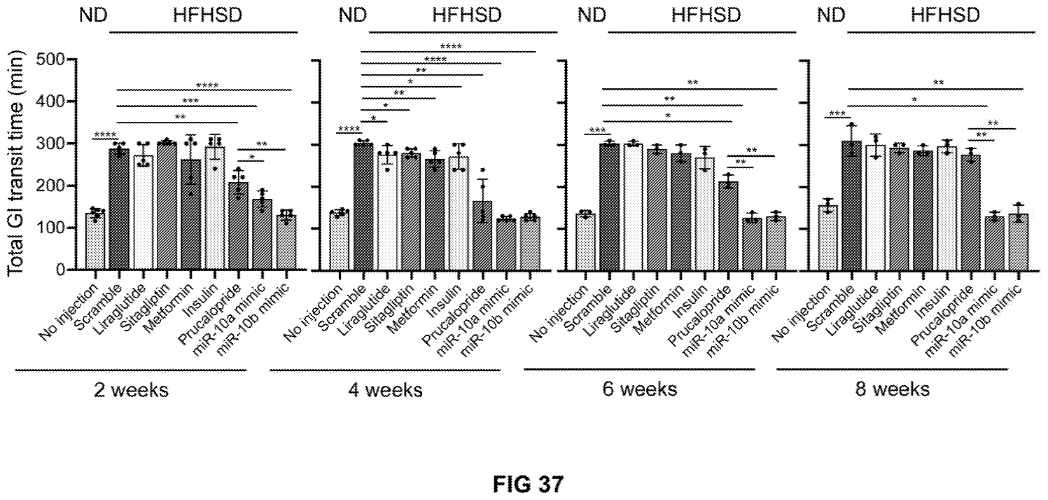

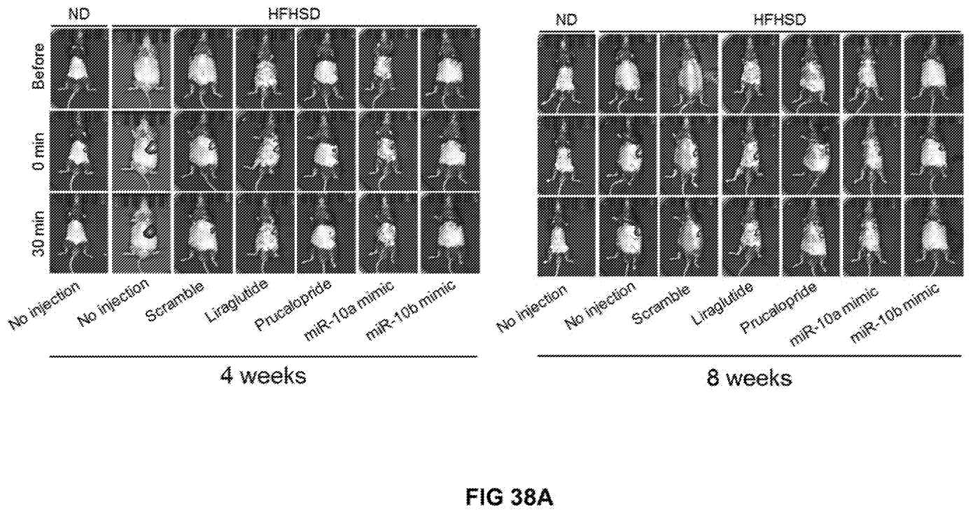

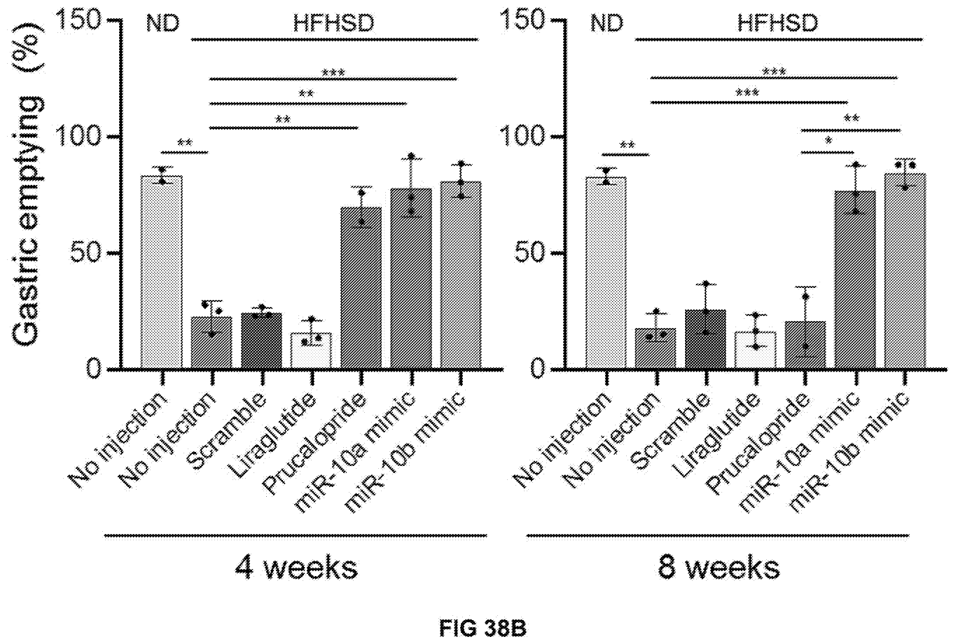

FIG. 35 illustrates a study design of drug effects of miR-10a-5p mimic and miR-10b-5p mimic, compared to popular anti-diabetic medications, and an FDA-approved prokinetic drug on blood glucose, body weight, and GI functions in diabetic mice fed a HFHSD over 8 weeks post treatment or healthy mice fed a ND without injection as a control. miR-10a-5p (500 ng/g body weight), miR-10b-5p (500 ng/g body weight), or scramble RNA (500 ng/g body weight) were injected twice, once at 1 week and once at 2 weeks by IP injection; Metformin (250 mg/kg body weight) or Sitagliptin (DPP4 inhibitor) (10 mg/kg body weight) was provided daily per oral (PO) for 4 weeks; Liraglutide (GLP-1 receptor agonist) (0.2 mg/kg body weight) was injected twice daily by subcutaneous injection (SC) injection for 2 weeks; Insulin (0.75 U/kg body weight) was injected once daily by IP injection for 4 weeks; Prucalopride (5-HT₄ receptor agonist) (2 mg/kg body weight) was provided daily PO for 4 weeks. Drug effects are shown in FIGS. 36-38. Treatment with miR-10a-5p mimic and miR-10b-5p mimic had better and longer effects in lowering (FIG. 36A) blood glucose and (FIG. 36B) body weight than the anti-diabetic medications and prokinetic drug described in FIG. 35. n=5 per group. Likewise, miR-10a-5p mimic and miR-10b-5p mimic have better and longer effects in improving GI motility than the anti-diabetic medications and Prucalopride that is used to treat chronic constipation (FIGS. 37-38). Total GI transit time was measured in each group at 2 weeks, 4 weeks, 6 weeks, and 8 weeks post treatment (FIG. 37). n=5 per group. *p<0.05, * *p<0.01, ***p<0.001, and ****p<0.0001. Drug treatment frequency is shown in FIG. 35. miR-10a-5p mimic, miR-10b-5p mimic, and Prucalopride, but not the four antidiabetic medications, improved total GI transit (FIG. 37). However, Prucalopride had a temporal effect in GI transit during treating for 4 weeks compared to miR-10a-5p mimic and miR-10b-5p mimic that have prolonged effect for 8 weeks. Finally, delayed gastric emptying in control scramble RNA HFHSD-fed diabetic mice was improved with miR-10a-5p mimic, miR-10b-5p mimic and Prucalopride at 4 weeks, while Liraglutide did not improve delayed gastric emptying (FIGS. 38A-38B). The normal gastric emptying rate was maintained in miR-10a-5p mimic and miR-10b-5p mimic injected mice at 8 weeks, but delayed in Prucalopride treated mice, suggesting that both miRNAs had much more prolonged effects in improving gastric emptying than Prucalopride. Drug treatment frequency is shown in FIG. 35. Together, these data demonstrate that treatment of diabetes with miR-10a-5p and/or miR-10b-5p mimics can provide clinical outcomes that are equivalent or better than established medications and promising investigational drugs.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating diabetes in a subject in need thereof, the method comprising administering to the subject an effective amount of a miR-10a-5p mimic, thereby treating the diabetic condition.

2. The method of claim 1, wherein the miR-10a-5p mimic is a miR-10a-5p duplex, a chemically modified double stranded miR-10a-5p, an unmodified double stranded miR-10a-5p, a single stranded chemically modified miR-10a-5p or a single stranded unmodified miR-10a-5p.

3. The method of claim 1, wherein the diabetes is type 2 diabetes.

4. The method of claim 1, wherein the miR-10a-5p mimic is mammalian.

5. The method of claim 1, wherein the miR-10a-5p mimic is human.

6. The method of claim 1, wherein the miR-10a-5p mimic is engineered.

7. The method of claim 1, wherein the miR-10a-5p mimic further comprises a pharmaceutically acceptable carrier or adjuvant.

8. A method of reducing body weight in a subject, the method comprising administering an effective amount of a miR-10a-5p mimic, thereby reducing body weight in the subject.

9. A method of lowering blood glucose in a subject, the method comprising administering an effective amount of a miR-10a-5p mimic, thereby lowering blood glucose in the subject.

10. A method for increasing insulin sensitivity comprising administering an effective amount of a miR-10a-5p mimic to a subject in need thereof, thereby increasing insulin sensitivity in the subject.

11. A method of treating diabetes-related fatty liver disease in a subject in need thereof comprising administering to the subject an effective amount of a miR-10a-5p mimic thereby treating the diabetes-related fatty liver disease.

12. A method of reducing diabetes-related inflammation in a subject in need thereof comprising administering to the subject an effective amount of a miR-10a-5p mimic thereby reducing the diabetes-related inflammation.

13. (canceled)

14. (canceled)

15. (canceled)

16. (canceled)

17. (canceled)

18. A method of treating gastrointestinal disease comprising administering an effective amount of a miR-10a-5p mimic to a subject in need thereof, thereby treating gastrointestinal disease in the subject.

19. (canceled)

20. (canceled)

21. (canceled)

22. (canceled)

23. (canceled)

24. (canceled)

25. (canceled)

26. (canceled)

27. The method of claim 1, wherein the miR-10a-5p mimic comprises a nucleic acid sequence comprising SEQ ID NO: 1, 2, 4, 5 or any combination thereof.

28. A method for increasing interstitial cells of Cajal (ICC) proliferation comprising administering an effective amount of a miR-10b-5p mimic to a subject in need thereof, thereby increasing ICC proliferation in the subject.

29. (canceled)

30. (canceled)

31. (canceled)

32. (canceled)

33. (canceled)

34. (canceled)

35. (canceled)

36. (canceled)

37. A method for increasing KIT expression in ICC comprising administering an effective amount of a miR-10b-5p mimic to a subject in need thereof, thereby increasing KIT expression in the subject.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method for increasing insulin sensitivity comprising administering an effective amount of a miR-10b-5p mimic to a subject in need thereof, thereby increasing insulin sensitivity in the subject.

52. A method for treating diabetes comprising administering an effective amount of a miR-10b-5p mimic to a subject in need thereof, thereby treating diabetes in the subject.

53. The method of claim 51, wherein the diabetes is type 1 or type 2 diabetes.

54. A method for increasing KIT expression in PSC or PPC by comprising administering an effective amount of a miR-10b-5p mimic to a subject in need thereof, thereby increasing KIT expression.

55. A method for increasing expression of INSR, IRS2 and IRS1 in skeletal muscle cells (SkMC) comprising administering an effective amount of a miR-10b-5p mimic to a subject in need thereof, thereby increasing INSR, IRS2 and IRS1 expression.

56. A method for reducing diabetes-related inflammation in a subject in need thereof comprising administering an effective amount of a miR-10b-5p mimic, thereby reducing the diabetes-related inflammation.

57. (canceled)

58. (canceled)

59. (canceled)

60. (canceled)

61. (canceled)

62. A composition comprising a miR-10b-5p mimic and a pharmaceutically acceptable carrier or adjuvant.

63. (canceled)

64. (canceled)

65. The method of claim 28, wherein the miR-10b-5p mimic comprises a nucleic acid sequence comprising SEQ ID NO: 6, 7, 8, 9, 10, 12, 13 or any combination thereof.


SEQUENCE LISTING (13 sequences)

SEQ ID NO: 1 - RNA, 23 nt, Artificial Sequence
  miRNA
  10a-5p mimic sense
uacccuguagauccgaauuugug

SEQ ID NO: 2 - RNA, 21 nt, Artificial Sequence
  miRNA
  10a-5p mimic antisense
augggacaucuaggcuuaaac

SEQ ID NO: 3 - RNA, 52 nt, Artificial Sequence
  miRNA
  Mouse miR-10a stem-loop
gaccugucugucuucuguauauacccuguagauccgaauuuguguaaggaau

SEQ ID NO: 4 - RNA, 52 nt, Artificial Sequence
  miRNA
  Human miR-10a stem-loop
gaucugucugucuucuguauauacccuguagauccgaauuuguguaaggaau

SEQ ID NO: 5 - RNA, 23 nt, Artificial Sequence
  miRNA
  10a Inhibitor
augggacaucuaggcuuaaacac

SEQ ID NO: 6 - RNA, 23 nt, Artificial Sequence
  miRNA
  10b mimic sense
uacccuguagaaccgaauuugug

SEQ ID NO: 7 - RNA, 21 nt, Artificial Sequence
  miRNA
  10b mimic antisense
augggacaucuuggcuuaaac

SEQ ID NO: 8 - RNA, 22 nt, Artificial Sequence
  miRNA
  10b 5p/3p antisense
auaaggggaucuuagcuuagac

SEQ ID NO: 9 - RNA, 62 nt, Artificial Sequence
  miRNA
  10b precursor
uacccuguagaaccgaauuugugugguacccacauagucacagauucgauucuaggggaaua

SEQ ID NO: 10 - RNA, 110 nt, Artificial Sequence
  miRNA
  Human miR-10b stem-loop
ccagagguuguaacguugucuauauauacccuguagaaccgaauuugugugguauccguauagucacagauucgauucuaggggaauauauggucgaugcaaaaacuuca

SEQ ID NO: 11 - RNA, 68 nt, Artificial Sequence
  miRNA
  Mouse miR-10b stem-loop
uauauacccuguagaaccgaauuugugugguacccacauagucacagauucgauucuaggggaauaua

SEQ ID NO: 12 - RNA, 23 nt, Artificial Sequence
  miRNA
  10b inhibitor
cacaaauucgguucuacagggua

SEQ ID NO: 13 - RNA, 22 nt, Artificial Sequence
  miRNA
  miR-10b-3p
cagauucgauucuaggggaaua